(19) United States
(12) Patent Application Publication
(10) Pub. No.: US 2009/0082358 A1
(43) Pub. Date: Mar. 26, 2009

(54) Vanilloid receptor ligands and their use in treatments

(76) Inventors: Nobuko Nishimura, West Hills, CA (US); Mark H. Norman, Thousand Oaks, CA (US); Nuria Tamayo, Newbury Park, CA (US); Phi Tang, Santa Ana, CA (US); Yunxin Y. Bo, Thousand Oaks, CA (US)

Correspondence Address: AMGEN INC., MAIL STOP 28-2-C, ONE AMGEN CENTER DRIVE, THOUSAND OAKS, CA, 91320-1799, US

(21) Appl. No.: 12/284,586

(22) Filed: Sep. 22, 2008

Related U.S. Application Data
(60) Provisional application No. 60/994,759, filed on Sep. 20, 2007.

(51) Int. Cl. A61K 31/4375; C07D 471/04; A61P 29/00; A61P 25/00; A61K 31/4985; A61K 31/519

(52) U.S. Cl. 514/249; 546/122; 544/350; 544/279; 514/264.1; 514/300

(57) ABSTRACT

Bicyclic 3,4-fused piperidine compounds, and compositions containing them, for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotizing agents, hair growth, vasomotor or allergic rhinitis, bronchial disorders or bladder disorders.

This application claims the benefit of U.S. Provisional Application No. 60/994,759, filed Sep. 20, 2007, which is hereby incorporated by reference.

### BACKGROUND

Cold sensation is derived from activation of the somatosensory system by a cold stimulus. Calcium imaging and patch clamp experiments in dissociated trigeminal and dorsal root ganglia neurons have revealed that cold stimuli induced calcium influx, suggesting the direct opening of a calcium-permeable ion channels by cold (Thut et al., 2003; Reid, 2005). A recently cloned non-selective cation channel, TRPM8 (transient receptor potential melastatin 8) or trp-p8 (identified as a prostate-specific gene, up-regulated in prostate cancer and other malignancies, (Tsavaler et al., 2001)) is activated by cold stimulus of 10 to 24° C. temperature (McKemy et al., 2002; Peier et al., 2002). In addition, TRPM8 is also activated by compounds that elicit cool sensation such as menthol, icilin (AG-3-5) (McKemy et al., 2002), and the endogenous lipid PIP₂ (Rohacs et al., 2005). Correlating with the cold sensitivity of both A delta and C-fibers, TRPM8 is highly expressed in sensory neurons of the trigeminal and dorsal root ganglia (McKemy et al., 2002; Peier et al., 2002; Thut et al., 2003). TRPM8 is also expressed in nerve fibers innervating urinary bladder in guinea pigs (Tsukimi et al., 2005) and humans (Mukerji et al., 2006) and believed to contribute to the bladder hypersensitivity.

Activation mechanism of TRPA1 by menthol and icilin appears to differ. Icilin requires calcium for robust activation of TRPM8, whereas menthol and cold do not (Chuang et al., 2004). Typically, activation by all these agonists follows a period of calcium-dependent desensitization. The domain swap analysis of chicken and rat TRPM8and further mutational studies revealed that determinants of icilin sensitivity map to a region of TRPM8 that corresponds to the capsaicin binding site in TRPV1transmembrane domain 3 to 4 region (Chuang et al., 2004).

Cold allodynia and mechanical hyperalgesia associated with neuropathic pain in humans and in rodent models of neuropathic and chemotherapy-induced pain. TRPM8 is shown to mediate the analgesia by agonists such as menthol and icilin (by desensitization of the receptor) during experimental neuropathic pain in rodents (Proudfoot et al., 2006). Further, attenuation of cold sensation and cold allodynia after chronic constriction injury model of neuropathic pain in TRPM8 knockout mice (Colburn et al., 2007; Dhaka et al., 2007) suggests that antagonists of TRPM8 may be considered as pain therapeutics for chemotherapy-induced pain, neuropathic pain and bladder disorders.

Mint oil that contains menthol, an agonist of TRPM8 has been reported to alleviate pain in post-herpetic neuralgia (Davies et al., 2002), a neuropathic pain condition. Furthermore, oral or intracerebroventricular injection of menthol decreased nociceptive responses to hot-plate test and acetic acid-induced writhing in mice (Galeotti et al., 2002). These responses are believed to be mediated by the activation and desensitization of the TRPM8. These observations and the knockout mice studies indicate that TRPM8 modulation by antagonists might be beneficial for patients experiencing neuropathic pain.

### SUMMARY

The present invention comprises a new class of compounds useful in the treatment of diseases, such as TRPM8-mediated diseases and other maladies, such as inflammatory or neuropathic pain and diseases involving sensory nerve function such as asthma, rheumatoid arthritis, osteoarthritis, inflammatory bowel disorders, urinary incontinence, migraine and psoriasis. In particular, the compounds of the invention are useful for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, anxiety, depression, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotizing agents, hair growth, vasomotor or allergic rhinitis, bronchial disorders or bladder disorders. Accordingly, the invention also comprises pharmaceutical compositions comprising the compounds, methods for the treatment of vanilloid-receptor-mediated diseases, such as inflammatory or neuropathic pain, asthma, rheumatoid arthritis, osteoarthritis, inflammatory bowel disorders, urinary incontinence, migraine and psoriasis diseases, using the compounds and compositions of the invention, and intermediates and processes useful for the preparation of the compounds of the invention.

The compounds of the invention are represented by the following general structure:

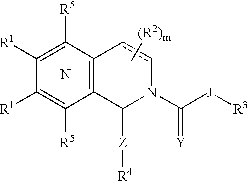

or a pharmaceutical acceptable salt thereof, wherein R¹, R², R³, R⁴, R⁵, J, Y and Z are defined below.

The foregoing merely summarizes certain aspects of the invention and is not intended, nor should it be construed, as limiting the invention in any way. All patents, patent applications and other publications recited herein are hereby incorporated by reference in their entirety.

### DETAILED DESCRIPTION

One aspect of the current invention relates to compounds having the general structure:

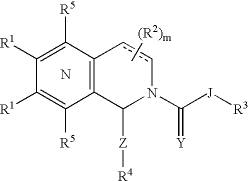

or any pharmaceutically-acceptable salt thereof, wherein:

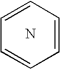

represents a six-membered heteroaryl ring containing 1 or 2 N atoms;

Y is NR^{a}, NCN, O or S;

Z is a direct bond, divalent C₁₋₄alk or divalent C₁₋₄haloalk;

[symbol] is a single bond or a double bond;

J is —N(R^{a})(CR^{c}R^{c})ₙ—, —O(CR^{c}R^{c})ₙ—, —S(CR^{c}R^{c})ₙ— or —(CR^{c}R^{c})ₙ—;

m is 0, 1 or 2;

n is 0, 1, 2 or 3;

R¹ is, independently in each instance, H, halo, C₁₋₆alk, C₁₋₆haloalk, NH₂, NHC₁₋₄alk, N(C₁₋₄alk)C₁₋₄alk or CN; or when attached to an N atom, R¹ is a lone pair of electrons;

R² is, independently in each instance, H, F, Cl, Br, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk or CN; or when attached to an N atom, R² is a lone pair of electrons;

R³ is C₁₋₈alk or a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the C₁₋₈alk and ring are substituted by 0, 1 or 2 oxo groups and the C₁₋₆alk and ring are additionally substituted by 0, 1, 2 or 3substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};

R⁴ is a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the ring is substituted by 0, 1 or 2 oxo groups and the ring is additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁴ is C₄₋₁₂alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁴ is 4-biphenyl substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};

R⁵ is H, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁵ is C₁₋₆alk or a saturated, partially saturated or unsaturated 5-, 6- or 7-membered ring containing 0, 1, 2, 3 or 4 atoms selected from N, O and S, wherein the C₁₋₆alk and ring are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}—OC(═O)R^{b}, —OC(═O)NR^{a}R^{a}, —OC₁₋₆alkNR^{a}R^{a}, —OC₂₋₆alkOR^{a}, —SR^{a}, —S(═O)R^{b}, —S(═O)₂R^{b}, —S(═O)₂NR^{a}R^{a},—NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};

R^{a} is independently, at each instance, H or R^{b}; and

R^{b} is independently, at each instance, phenyl, benzyl or C₂₋₆alk, the phenyl, benzyl and C₁₋₄alk being substituted by 0, 1, 2 or 3 substituents selected from halo, C₁₋₄alk, C₁₋₃haloalk, —OC₁₋₄alk, —NH₂, —NHC₁₋₄alk, and —N(C₁₋₄alk)C₁₋₄alk;

R^{c} is independently, at each instance, H, halo, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk.

Another aspect of the current invention relates to compounds having the general structure:

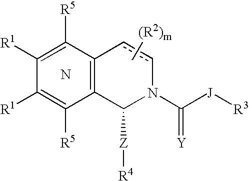

or any pharmaceutically-acceptable salt thereof, wherein:

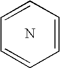

represents a six-membered heteroaryl ring containing 1 or 2 N

atoms;

Y is NR^{a}, NCN, O or S;

Z is a direct bond, divalent C₁₋₄alk or divalent C₁₋₄haloalk;

[symbol] is a single bond or a double bond;

J is —N(R^{a})(CR^{c}R^{c})ₙ—, —O(CR^{c}R^{c})ₙ—, —S(CR^{c}R^{c})ₙ— or —(CR^{c}R^{c})ₙ—;

m is 0, 1 or 2;

n is 0, 1, 2 or 3;

R¹ is, independently in each instance, H, halo, C₁₋₆alk, C₁₋₆haloalk, NH₂, NHC₁₋₄alk, N(C₁₋₄alk)C₁₋₄alk or CN; or when attached to an N atom, R¹ is a lone pair of elections;

R² is, independently in each instance, H, F, Cl, Br, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk or CN; or when attached to an N atom, R² is a lone pair of electrons;

R³ is C₁₋₈alk or a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the C₁₋₆alk and ring are substituted by 0, 1 or 2 oxo groups and the C₁₋₆alk and ring are additionally substituted by 0, 1, 2 or 3substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{h}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};

R⁴ is a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the ring is substituted by 0, 1 or 2 oxo groups and the ring is additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆)alkOR^{a}; or R⁴ is C₄₋₁₂alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{h}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};

R⁵ is H, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁵ is C₁₋₆alk or a saturated, partially saturated or unsaturated 5-, 6- or 7-membered ring containing 0, 1, 2, 3 or 4 atoms selected from N, O and S, wherein the C₁₋₆alk and ring are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₁₋₄alkOR^{a};

R^{a} is independently, at each instance, H or R^{b}; and

R^{b} is independently, at each instance, phenyl, benzyl or C₁₋₆alk, the phenyl, benzyl and C₁₋₆alk being substituted by 0, 1, 2 or 3 substituents selected from halo, C₁₋₄alk, C₁₋₃haloalk, —OC₁₋₄alk, —NH₂, —NHC₁₋₄alk, and —N(C₁₋₄alk)C₁₋₄alk;

R^{c} is independently, at each instance, H, halo, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk.

In another embodiment, in conjunction with any of the above or below embodiments, J is N, O or CH₂.

In another embodiment, in conjunction with any of the above or below embodiments,

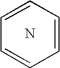

represents a six-membered heteroaryl ring containing 1 N atom.

In another embodiment, in conjunction with any of the above or below embodiments,

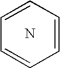

represents a six-membered heteroaryl ring containing 2 N atoms.

In another embodiment, in conjunction with any of the above or below embodiments,

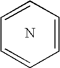

represents

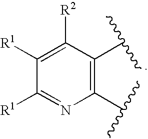

In another embodiment, in conjunction with any of the above or below embodiments,

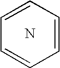

represents

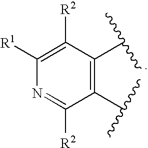

In another embodiment, in conjunction with any of the above or below embodiments,

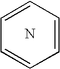

represents

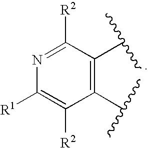

In another embodiment, in conjunction with any of the above or below embodiments,

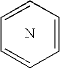

represents

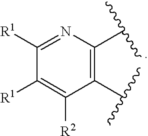

In another embodiment, in conjunction with any of the above or below embodiments,

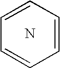

represents

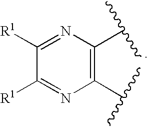

In another embodiment, in conjunction with any of the above or below embodiments,

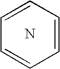

represents

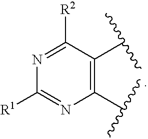

In another embodiment, in conjunction with any of the above or below embodiments,

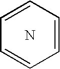

represents

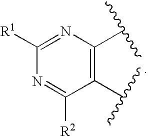

In another embodiment, in conjunction with any of the above or below embodiments, R¹ is H; or when attached to an N atom, R¹ is a lone pair of electrons.

In another embodiment, in conjunction with any of the above or below embodiments, R³ is C₁₋₈alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R³ is C₁₋₈alk.

In another embodiment, in conjunction with any of the above or below embodiments, R³ is phenyl substituted by 0, 1, 2 or 3 substituents selected from C₁₋₈alk, C₁₋₄haloalk, halo, cyano, nitro, —C(═O)R^{b}, —C(O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R³ is phenyl or benzyl, both of which are substituted by 0, 1, 2 or 3substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NRⁿ)NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R³ is pyridyl or pyrimidinyl, both of which are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁴ is phenyl substituted by 1, 2 or 3 substituents selected from C₁₋₄alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)R^{b}, —N(R^{a})S(═O)₂NR^{a}R^{a}, —NR^{a}C₂₋₆alkNR^{a}R^{a} and —NRⁿC₂₋₆alkOR^{a}.

In another embodiment, in conjunction with any of the above or below embodiments, R⁴ is phenyl substituted in para position by one substituent selected from C₁₋₄alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁴ saturated, partially saturated or unsaturated 5-, 6- or 7-membered monocyclic or 8, 9, 10 or 11-membered bicyclic ring containing 1, 2, 3 or 4 atoms selected from N, O and S, wherein the ring is substituted by 0, 1 or 2 oxo groups and the ring is additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}Rⁿ, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁴ pyridine or pyrimidine both of which are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁴ is C₄₋₁₂alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁴ is 4-trifluoromethylphenyl.

In another embodiment, in conjunction with any of the above or below embodiments, R⁴ is 4-C₁₋₆alkphenyl.

In another embodiment, in conjunction with any of the above or below embodiments, R⁴ is 4-diC₁₋₄alkaminophenyl.

In another embodiment, in conjunction with any of the above or below embodiments, R⁴ is 4-C₁₋₄alk-O-phenyl.

In another embodiment, in conjunction with any of the above or below embodiments, R⁵ is II, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}Rⁿ, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁵ is H or F.

In another embodiment, in conjunction with any of the above or below embodiments, R⁵ is H.

In another embodiment, in conjunction with any of the above or below embodiments, R⁵is C₁₋₆alk or a saturated, partially saturated or unsaturated 5-, 6- or 7-membered ring containing 0, 1, 2.3 or 4 atoms selected from N, O and S, wherein the C₁₋₆alk and ring are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}Rⁿ, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};

In another embodiment, in conjunction with any of the above or below embodiments, Z is a direct bond.

Another aspect of the invention relates to a method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, comprising the step of administering a compound according to claim 1.

Another aspect of the invention relates to a pharmaceutical composition comprising a compound according to claim 1 and a pharmaceutically-acceptable diluent or carrier.

Another aspect of the invention relates to the use of a compound according to any of the above embodiments as a medicament.

Another aspect of the invention relates to the use of a compound according to any of the above embodiments in the manufacture of a medicament for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, anxiety, depression, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders.

The compounds of this invention may have in general several asymmetric centers and are typically depicted in the form of racemic mixtures. This invention is intended to encompass racemic mixtures, partially racemic mixtures and separate enantiomers and diasteromers.

Unless otherwise specified, the following definitions apply to terms found in the specification and claims:

“C_{α-β}alk” means an alkyl group comprising a minimum of a and a maximum of β carbon atoms in a branched, cyclical or linear relationship or any combination of the three, wherein α and β represent integers. The alkyl groups described in this section may also contain one or two double or triple bonds. A designation of C₀alk indicates a direct bond. Examples of C₁₋₆alkyl include, but are not limited to the following:

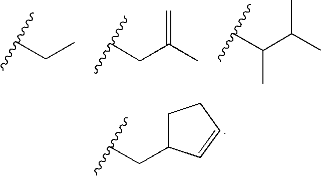

“Benzo group”, alone or in combination, means the divalent radical C₄H₄=, one representation of which is —CH═CH—CH═CH—, that when vicinally attached to another ring forms a benzene-like ring—for example tetrahydronaphthylene, indole and the like. The terms “oxo” and “thioxo” represent the groups ═O (as in carbonyl) and ═S (as in thiocarbonyl), respectively.

“Halo” or “halogen” means a halogen atoms selected from F, Cl, Br and I.

“C_{V-W}haloalk” means an alk group, as described above, wherein any number—at least one—of the hydrogen atoms attached to the alk chain are replaced by F, CI, Br or I. The group N(R^{a})R^{a} and the like include substituents where the two R^{a} groups together form a ring, optionally including a N, O or S atom, and include groups such as:

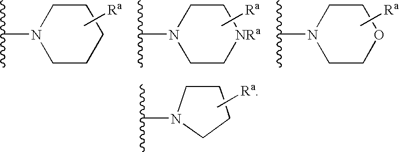

The group N(C_{α-β}alk)C_{α-β}alk, wherein α and β are as defined above, include substituents where the two C_{α-β}alk groups together form a ring, optionally including a N, O or S atom, and include groups such as:

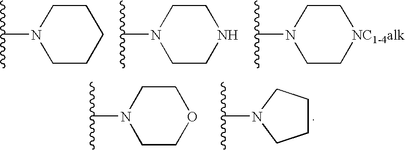

“Heterocycle” means a ring comprising at least one carbon atom and at least one other atom selected from N, O and S. Examples of heterocycles that may be found in the claims include, but are not limited to, the following:

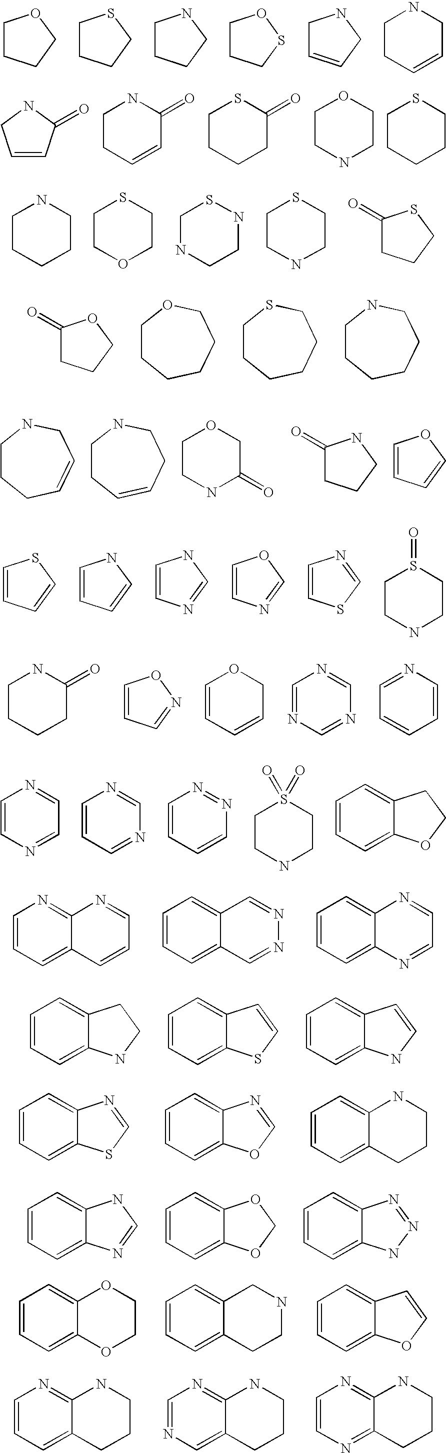

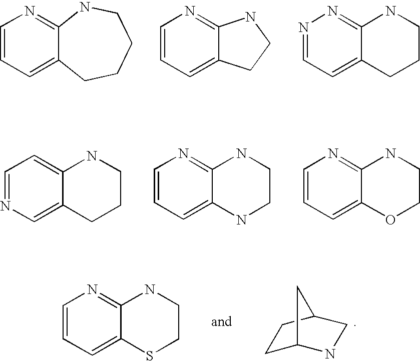

“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

“Saturated, partially-saturated or unsaturated” includes substituents saturated with hydrogens, substituents completely unsaturated with hydrogens and substituents partially saturated with hydrogens.

“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, Inflates, tosylates and the like. Preferred leaving groups are indicated herein where appropriate.

“Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl, Silylation of an amino groups provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl. 4-methoxyphenylmethyl and the like, can be removed under hydrolysis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′═O, S, NR), and the like, which are illustrated in the following examples:

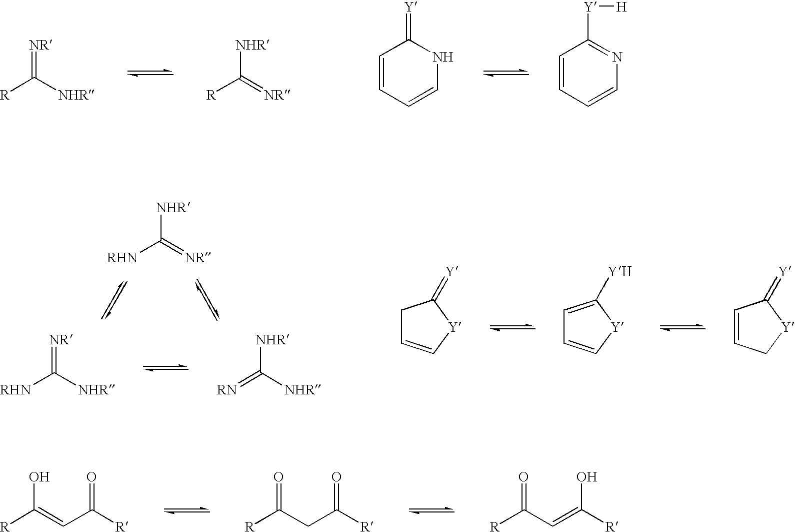

and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

The specification and claims contain listing of species using the language “selected from . . . and . . . ” and “is . . . or . . . ” (sometimes referred to as Markush groups). When this language is used in this application, unless otherwise slated it is meant to include the group as a whole, or any single members thereof, or any subgroups thereof. The use of this language is merely for shorthand purposes and is not meant in any way to limit the removal of individual elements or subgroups as needed.

### EXPERIMENTAL

Unless otherwise noted, all materials were obtained from commercial suppliers and used without further purification. All parts are by weight and temperatures are in degrees centigrade unless otherwise indicated. All microwave assisted reactions were conducted with a Smith Synthesizer from Biotage. All compounds showed NMR spectra consistent with their assigned structures. Melting points were determined on a Buchi apparatus and are uncorrected. Mass spectral data was determined by electrospray ionization technique. All examples were purified to >90% purity as determined by high-performance liquid chromatography. Unless otherwise stated, reactions were run at room temperature.

The following abbreviations are used:
DCM dichloromethane
DMSO—dimethyl sulfoxide
DMF—N,N-dimethylformamide
THF—tetrahydrofuran
Et₂₀—diethyl ether
EtOAc—ethyl acetate
MeOH—methyl alcohol
EtOH—ethyl alcohol
IPA—isopropyl alcohol
MeCN—acetonitrile
MeI—iodomethane
NMP —1-methyl-2-pyrrolidinone
DCM—dichloromethane
TFA—trifluoroacetic acid
MTBE—methyl tert-butyl ether
DIPEA—diisopropylethyl amine
HBTU—2-(1H-Benzotriazole-1-yl)-1,1,3,3-tetramethylaminium hexafluorophosphate
HATU—O-(7-Azobenzotriazol-1-yl)-1,1,3,3-tetramethyluronium hexafluorophosphate
Sat.—saturated
h—hour
min—minutes
mL milliliters
g grams
mg milligrams

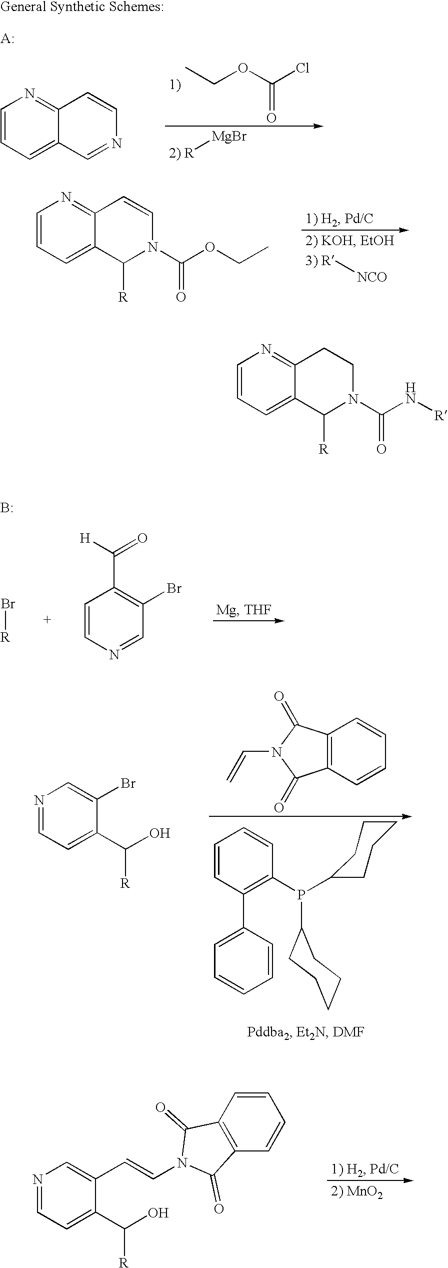

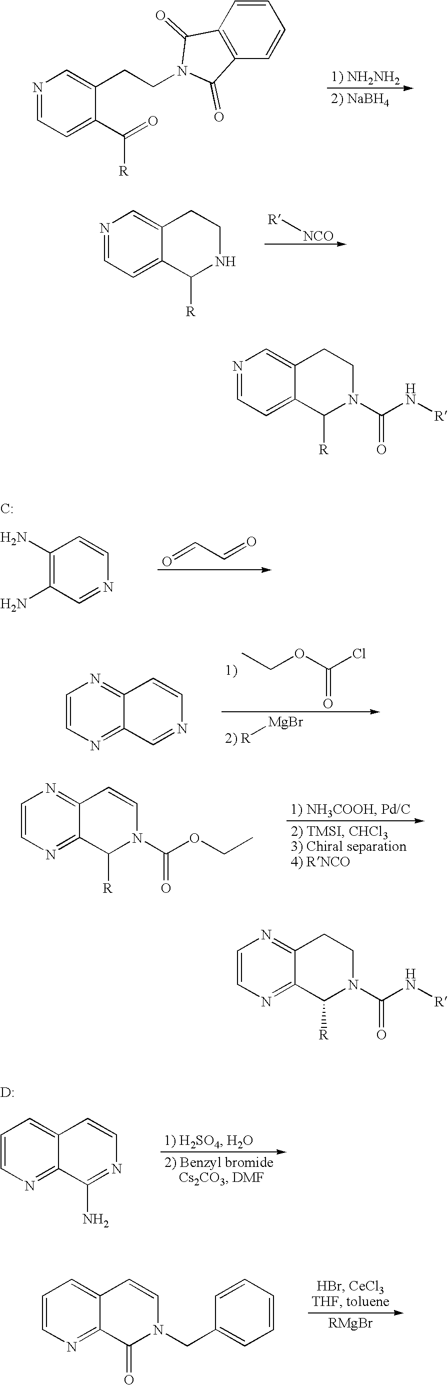

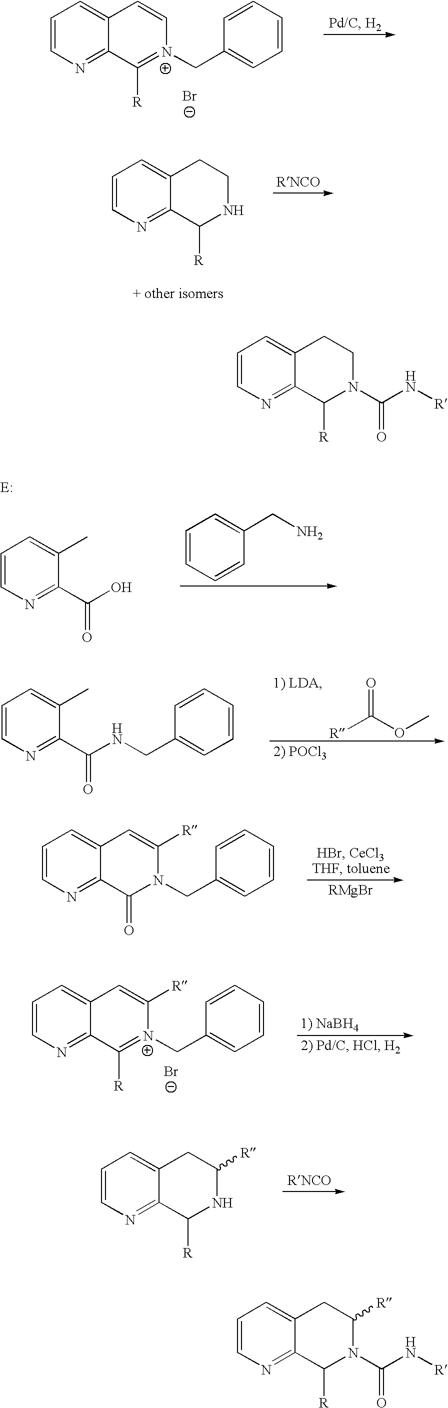

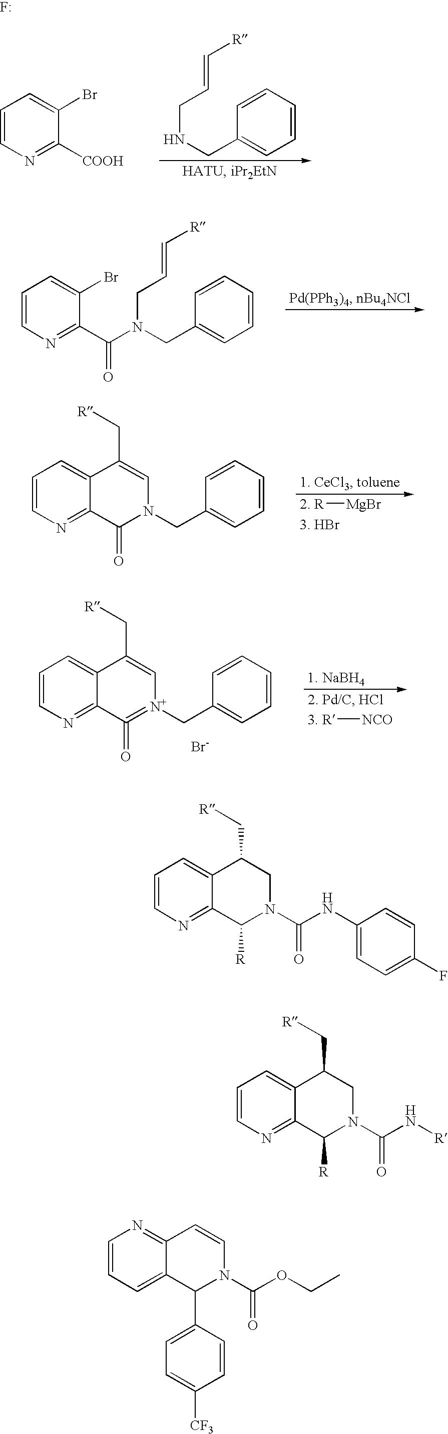

### Example 1

### Ethyl 5-(4-(trifluoromethyl)phenyl)-1,6-naphthyridine-6(5H)-carboxylate

The 4-trifluoromethylphenyl Grignard reagent was prepared by adding 1-bromo-4-(trifluoromethyl)benzene (1.5 mL, 10.8 mmol) to a suspension of magnesium turnings (261 mg, 10.7 mmol) and catalytic amount of iodine in THF (10 mL) at room temperature. A different round-bottomed flask containing 1,6-naphthyridine (1.0 g, 7.7 mmol) in anhydrous THF (10 mL) was charged with ethyl chloro-formate (0.73 mL, 7.7 mmol) under a stream of N2 and the mixture was stirred at room temperature for 15 minutes, and then cooled to 0° C. The previously made Grignard reagent was then cannulated into this solution dropwise and the reaction mixture was stirred for 1 h at 0° C. followed by 1 h at room temperature. This mixture was quenched with saturated NH₄Cl and extracted with EtOAc. The organic layer was washed with brine, dried over MgSO₄, filtered, and concentrated in vacuo. The crude product was purified by silica gel chromatography (20-30% EtOAc in hexanes) to give ethyl 5-(4-(trifluoromethyl)phenyl)-1,6-naphthyridine-6(5H)-carboxylate as an orange oil. MS (ESI pos. ion) m/z: 349 (M+1). ¹H NMR (400 MHz, DMSO-d₆): δ 8.38 (d, J= 3.7 Hz, 1H), 7.83 (br s, 1H), 7.79 (d, J= 8.2 Hz, 2H), 7.55 (d, J= 7.6 Hz, 2H), 7.35 (br s, 1H), 7.19 (dd, J=7.2 Hz, 5.1 Hz, 1H), 6.66 (s, 1H), 6.85 (d, J= 6.8 Hz, 2H), 4.18-4.20 (m, 2H), 1.17-1.25 (m, 3H).

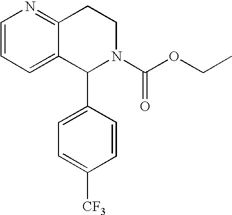

### Example 2

### Ethyl 5-(4-(trifluoromethyl)phenyl)-7,8-dihydro-1,6-naphthyridine-6(5H)-carboxylate

A solution of ethyl 5-(4-(trifluoromethyl)phenyl)-1,6-naphthyridine-6(5H)-carboxylate (1.7 g, 4.8 mmol) in EtOH (20 mL) was stirred with 10% Pd/C (0.5 g, 4.8 mmol) under hydrogen atmosphere at room temperature for 1 h. The reaction mixture was filtered through a celite pad and the filtrate was concentrated in vacuo to provide the title compound (1.3 g) as yellow oil. The crude product was used for the next step. MS (ESI pos. ion) m/z: 351 (M+1).

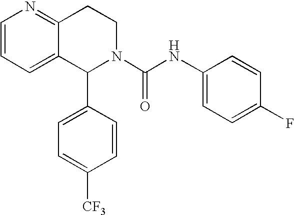

### Example 3

### N-(4-Fluorophenyl)-5-(4-(trifluoromethyl)phenyl)-7,8-dihydro-1,6-naphthyridine-6(5H)-carboxamide

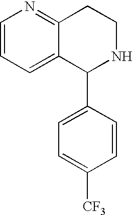

### Step 1. 5-(4-(Trifluoromethyl)phenyl)-5,6,7,8-tetrahydro-1,6-naphthyridine

A 250-mL, round-bottomed flask was charged with potassium hydroxide (10.2 g, 182.4 mmol), EtOH (100 mL), and the resulting suspension was heated to 80° C. After the potassium hydroxide was dissolved, crude ethyl 5-(4-(trifluoromethyl)-phenyl)-7,8-dihydro-1,6-naphthyridine-6(5H)-carboxylate (1.3 g, 3.6 mmol) was added and the solution was heated at 90° C. for 30 h. The mixture was allowed to cool to room temperature. The solvent was partially removed in vacuo and the residue was diluted with EtOAc. The organic phase was washed with water, brine, dried over MgSO₄, filtered, and concentrated in vacuo. The resulting residue was purified by silica gel chromatography (5% MeOH in DCM) to give 5-(4-(trifluoromethyl)phenyl)-5,6,7,8-tetrahydro-1,6-naphthyridine as an off-white solid. MS (ESI pos. ion) m/z: 279 (M+1). ¹H NMR (400 MHz, DMSO-d₆): δ 8.35 (d, J= 4.0 Hz, 2H), 7.70 (d, J= 8.0 Hz, 2H), 7.50 (d, J= 7.5 Hz, 2H), 7.07 (m, 1H), 7.01 (m, 1H), 3.10-3.18 (m, 2H), 2.97-3.06 (m, 2H), 2.18-2.85 (m, 1H).

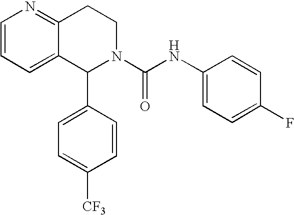

### Step 2. N-(4-Fluorophenyl)-5-(4-(trifluoromethyl)phenyl)-7,8-dihydro-1,6-naphthyridine-6(5H)-carboxamide

A solution of 5-(4-(trifluoromethyl)phenyl)-5,6,7,8-tetrahydro-1,6-naphthyridine (170 mg, 0.6 mmol) in 1,2-dichloroethane (5 mL) was treated with 4-fluorophenyl isocyanate (0.08 mL, 0.7 mmol) and the mixture was stirred at room temperature for 1 h. The solvent was removed in vacuo and the residue was purified by silica gel chromatography (2-3% MeOH in DCM) to give N-(4-fluorophenyl)-5-(4-(trifluoromethyl)phenyl)-7,8-dihydro-1,6-naphthyridine-6(5H)-carboxamide as a white solid. MS (ESI pos. ion) m/z: 416 (M+1). ¹H NMR (400 MHz, DMSO-d₆): δ 8.78 (s, 1H), 8.49 (d, J= 4.5 Hz, 1H), 7.71 (d, J= 8.5 Hz, 2H), 7.53 (d, J=7.5 Hz, 1H), 7.50 (dd, J= 8.8 Hz, 4.8 Hz, 2H), 7.44 (d, J= 8.0 Hz, 2H), 7.29 (dd, J=7.5 Hz, 4.5 Hz, 1H), 7.09 (t, J= 8.8 Hz, 2H), 6.71 (s, 1H), 4.14 (dd, J= 8.8 Hz, 4.3 Hz, 1H), 3.36-3.43 (m, 1H), 3.07-3.14 (m, 1H), 2.92-2.96 (m, 1H). Purification of racemic N-(4-Fluorophenyl)-5-(4-(trifluoromethyl)phenyl)-7,8-dihydro-1,6-naphthyridine-6(5H)-carboxamide on chiral SFC using the following conditions (Chiralcel AD-H (250×21 Mm), 45% methanol/CO₂ (100 bar), 65 ml/min, 220 nm) provided:

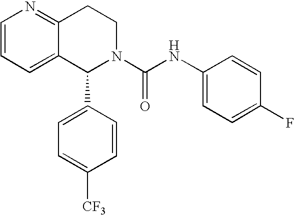

### Example 4

(5R)—N-(4-Fluorophenyl)-5-(4-(trifluoromethyl)phenyl)-7,8-dihydro-1,6-naphthyridine-6(5H)-carboxamide as a white solid (retention time= 0.83 min. Chiralcel AD-H (150×0.46 cm), 35% methanol/CO₂ (100 bar), 4 ml/min, 220 nm), MS (ESI pos. ion) m/z: 416 (M+1).

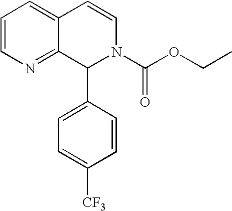

### Example 5

### Ethyl 8-(4-(trifluoromethyl)phenyl)-1,7-naphthyridine-7(8H)-carboxylate

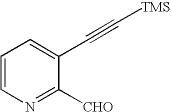

### Step 1. 3-(2-(Trimethylsilyl)ethynyl)picolinaldehyde

A 50-mL, round-bottomed flask was charged with 3-bromopicolinaldehyde (2.0 g, 10.6 mmol), dichlorobis(triphenylphosphine)palladium(II) (372 mg, 0.53 mmol), copper(I) iodide (101 mg, 0.53 mmol), and DMF (10 mL). The resulting suspension was treated with triethylamine (1.5 mL, 10.6 mmol), followed by (trimethylsilyl)acetylene (2.6 mL, 19.1 mmol). The reaction mixture was stirred at room temperature for 1.5 h and diluted with EtOAc. The organic layer was washed with water, brine, dried over Na₂SO₄; and concentrated in vacuo. The resulting residue was purified by silica gel chromatography (10-20% EtOAc in hexanes) to give 3-(2-(trimethylsilyl)ethynyl)picolinaldehyde (1.8 g, 85%) as a colorless oil. MS (ESI pos. ion) m/z: 204 (M+1).

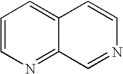

### Step 2,1,7-Naphthyridine

A solution of 3-(2-(trimethylsilyl)ethynyl)picolinaldehyde (1.8 g, 9.0 mmol) in EtOH (40 mL) was saturated with ammonia. The solution was heated at 80° C. for 2 h in a sealed tube and cooled to room temperature. The solvent was removed in vacuo and the residue was diluted with EtOAc, washed with saturated NaHCO₃, water, brine, dried over Na₂SO₄; and concentrated in vacuo. The crude product was purified by silica gel chromatography (20% EtOAc in hexanes) to give 1,7-naphthyridine as a brownish solid. MS (ESI pos. ion) m/z: 131 (M+1)

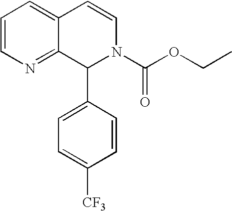

### Step 3. Ethyl 8-(4-(trifluoromethyl)phenyl)-1,7-naphthyridine-7(8H)-carboxylate

The 4-trifluoromethylphenyl Grignard reagent was prepared by adding 1-bromo-4-(trifluoromethyl)benzene (0.8 mL, 5.5 mmol) to a suspension of magnesium turnings (134 mg, 5.5 mmol) and catalytic amount of iodine in THF (5 mL) at room temperature. A different round-bottomed flask containing 1,7-naphthyridine (552 mg, 4.2 mmol) in anhydrous THF (5 mL) was charged with ethyl chloroformate (0.45 mL, 4.7 mmol) under a stream of N2 and the mixture was stirred at room temperature for 15 minutes, and then cooled to 0° C. The previously made Grignard reagent was then cannulated into this solution dropwise and the reaction mixture was stirred for 1 h at 0° C. followed by 1 h at room temperature. This mixture was quenched with saturated NH₄Cl and extracted with EtOAc. The organic layer was washed with brine, dried over MgSO₄, filtered, and concentrated in vacuo. The crude product was purified by silica gel chromatography (20-30% EtOAc in hexanes) to provide ethyl 8-(4-(trifluoromethyl)phenyl)-1,7-naphthyridine-7(8H)-carboxylate as a yellow oil. MS (ESI pos. ion) m/z: 349 (M+1). ¹H NMR (400 MHz, DMSO-d₆): δ 8.36 (d, J=3.9 Hz, 1H), 7.68 (d, J= 8.2 Hz, 2H), 7.59 (d, J= 6.7 Hz, 1H), 7.51 (d, J= 8.0 Hz, 2H), 7.28 (dd, J= 7.5 Hz, 4.8 Hz, 1H), 7.18 (br s, 1H), 6.41 (s, 1H), 6.00 (br s, 1H), 4.19-4.21 (m, 2H), 1.17-1.25 (m, 3H).

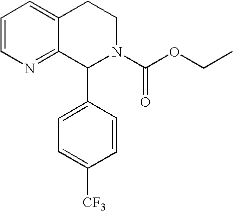

### Example 6

### Ethyl 8-(4-(trifluoromethyl)phenyl)-5,6-dihydro-1,7-naphthyridine-7(8H)-carboxylate

A solution of ethyl 8-(4-(trifluoromethyl)phenyl)-1,7-naphthyridine-7(8H)-carboxylate (977 mg, 2.8 mmol) in EtOH (10 mL) was stirred with 10% Pd/C (0.3 g, 2.8 mmol) under hydrogen atmosphere at room temperature for 1 h. The reaction mixture was filtered through a celite pad and the filtrate was concentrated in vacuo to provide ethyl 8-(4-(trifluoromethyl)phenyl)-5,6-dihydro-1,7-naphthyridine-7(8H)-carboxylate (981 mg, 100%) as a colorless oil. MS (ESI pos. ion) m/z: 351 (M+1).

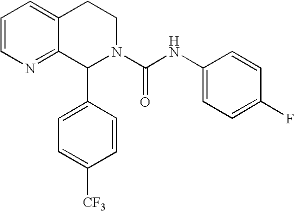

### Example 7

### N-(4-Fluorophenyl)-8-(4-(trifluoromethyl)phenyl)-5,6-dihydro-1,7-naphthyridine-7(8H)-carboxamide

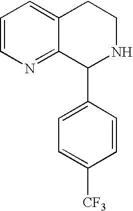

### Step 1. 8-(4-(Trifluoromethyl)phenyl)-5,6,7,8-tetrahydro-1,7-naphthyridine

A 250-mL, round-bottomed flask was charged with potassium hydroxide (7.4 g, 131.3 mmol) and EtOH (50 mL), the suspension was heated to 80° C. After the potassium hydroxide was dissolved, ethyl 8-(4-(trifluoromethyl)phenyl)-5,6-dihydro-1,7-naphthyridine-7(8H)-carboxylate (0.92 g, 2.6 mmol) was added and the solution was heated at 90° C. for 30 h. The mixture was allowed to cool to room temperature. The solvent was partially removed in vacuo and the residue was diluted with EtOAc. The organic phase was washed with water, brine, dried over MgSO₄, filtered, and concentrated in vacuo. The resulting residue was purified by silica gel chromatography (5% MeOH in DCM) to give 8-(4-(trifluoromethyl)phenyl)-5,6,7,8-tetrahydro-1,7-naphthyridine as an off-white solid. MS (ESI pos. ion) m/z: 279 (M+1). ¹H NMR (400 MHz, DMSO-d₆): δ 8.35 (d, J= 4.0 Hz, 2H), 7.70 (d, J= 8.0 Hz, 2H), 7.50 (d, J= 7.5 Hz, 2H), 7.07 (m, 1H), 7.01 (m, 1H), 3.10-3.18 (m, 2H), 2.97-3.06 (m, 2H), 2.18-2.85 (m, 1H).

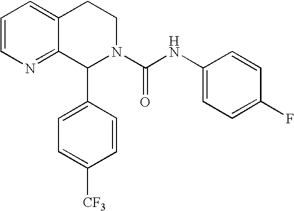

### Step 2. N-(4-Fluorophenyl)-8-(4-(trifluoromethyl)phenyl)-5,6-dihydro-1,7-naphthyridine-7(8H)-carboxamide

A solution of 8-(4-(trifluoromethyl)phenyl)-5,6,7,8-tetrahydro-1,7-naphthyridine (252 mg, 0.91 mmol) in 1,2-dichloroethane (5 mL) was treated with 4-fluorophenyl isocyanate (0.1 mL, 0.91 mmol) and the mixture was stirred at room temperature for 1 h. The solvent was removed in vacuo and the residue was purified by silica gel chromatography (30-40% EtOAc in hexanes) to give N-(4-fluorophenyl)-8-(4-(trifluoromethyl)phenyl)-5,6-dihydro-1,7-naphthyridine-7(8H)-carboxamide as a white solid. MS (ESI pos. ion) m/z: 416 (M+1). ¹H NMR (400 MHz, DMSO-d₆): 8.81 (s, 1H), 8.47 (dd, J= 4.6 Hz, 1.5 Hz, 1H), 7.68-7.73 (m, 3H), 7.45-7.51 (m, 4H), 7.33 (dd, J= 7.6 Hz, 4.7 Hz, 1H), 7.05-7.12 (m, 2H), 6.59 (s, 1H), 4.10-4.15 (m, 1H), 3.34-3.37 (m, 1H), 3.01-3.09 (m, 1H), 2.81-2.87 (m, 1H).

Purification of racemic N-(4-fluorophenyl)-8-(4-(trifluoromethyl)phenyl)-5,6-dihydro-1,7-naphthyridine-7(8H)-carboxamide on chiral SFC using the following conditions (Chiralcel OD-H (3×25 cm), 25% methanol/CO₂ (100 bar), 50 ml/min, 220 nm) provided:

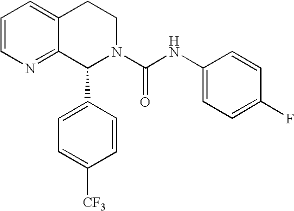

### Example 8

(R)—N-(4-Fluorophenyl)-8-(4-(trifluoromethyl)phenyl)-5,6-dihydro-1,7-naphthyridine-7(8H)-carboxamide as a white solid (retention time= 2.1 min. Chiralcel OD-H (25×0.46 cm), 25% methanol/CO₂ (100 bar), 3 ml/min, 220 nm), MS (ESI pos. ion) m/z: 416 (M+1).

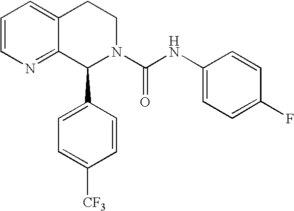

### Example 9

(S)—N-(4-Fluorophenyl)-8-(4-(trifluoromethyl)phenyl)-5,6-dihydro-1,7-naphthyridine-7(8H)-carboxamide as a white solid (retention time= 4.3 min. Chiralcel OD-H (25×0.46 cm), 25% methanol/CO₂ (100 bar), 3 ml/min, 220 nm), MS (ESI pos. ion) m/z: 416 (M+1).

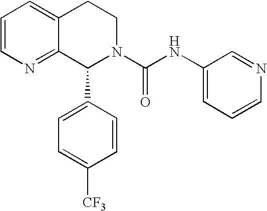

### Example 10

### (R)—N-(pyridin-3-yl)-8-(4-(trifluoromethyl)phenyl)-5,6-dihydro-1,7-naphthyridine-7(8H)-carboxamide

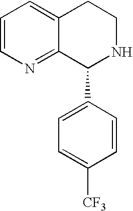

### Step 1. (R)-8-(4-(Trifluoromethyl)phenyl)-5,6,7,8-tetrahydro-1,7-naphthyridine

Purification of racemic 8-(4-(trifluoromethyl)phenyl)-5,6,7,8-tetrahydro-1,7-naphthyridine (prepared as shown in example 7, step 1) on chiral SFC using the following conditions (Chiralcel AD-H (250×21 mm), 15% ethanol/CO₂ (100 bar), 65 ml/min, 220 nm) provided (R)-8-(4-(Trifluoromethyl)phenyl)-5,6,7,8-tetrahydro-1,7-naphthyridine as a white solid (retention time= 2.5 min. Chiralcel AD-H (125×0.46 cm), 10% methanol/CO₂ (100 bar), 4 ml/min, 220 nm), MS (ESI pos. ion) m/z: 279 (M+1)

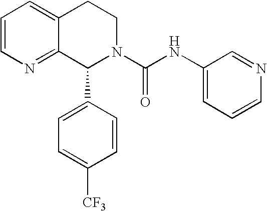

### Step 2. (R)—N-(pyridin-3-yl)-8-(4-(trifluoromethyl)phenyl)-5,6-dihydro-1,7-naphthyridine-7(8H)-carboxamide

A solution of (R)-8-(4-(trifluoromethyl)phenyl)-5,6,7,8-tetrahydro-1,7-naphthyridine (60 mg, 0.22 mmol) in DCM (10 mL) was treated with 3-isocyanatopyridine (28 mg, 0.24 mmol) and the mixture was stirred at room temperature for 2 h. The solvent was removed in vacuo and the residue was purified by silica gel chromatography (0-10% MeOH in DCM) to give (R)—N-(pyridin-3-yl)-8-(4-(trifluoromethyl)phenyl)-5,6-dihydro-1,7-naphthyridine-7(8H)-carboxamide as an off-white solid. MS (ESI pos. ion) m/z: 399 (M+1).

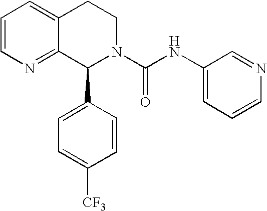

### Example 11

### (S)—N-(pyridin-3-yl)-8-(4-(trifluoromethyl)phenyl)-5,6-dihydro-1,7-naphthyridine-7(8H)-carboxamide

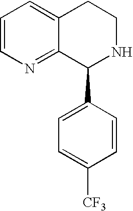

### Step 1. (S)-8-(4-(trifluoromethyl)phenyl)-5,6,7,8-tetrahydro-1,7-naphthyridine

Purification of racemic 8-(4-(1trifluoromethyl)phenyl)-5,6,7,8-tetrahydro-1,7-naphthyridine (prepared as shown in example 7, step 1) on chiral SFC using the following conditions (Chiralcel AD-H (250×21 mm), 15% ethanol/CO₂ (100 bar), 65 ml/min, 220 nm) provided (S)-8-(4-(trifluoromethyl)phenyl)-5,6,7,8-tetrahydro-1,7-naphthyridine as a white solid (retention time= 3.6 min. Chiralcel AD-H (125×0.46 cm), 10% methanol/CO₂ (100 bar), 4 ml/min, 220 nm), MS (ESI pos. ion) m/z: 279 (M+1).

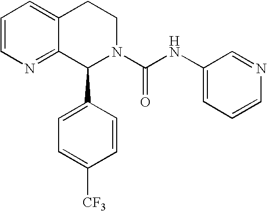

### Step 2. (S)—N-(pyridin-3-yl)-8-(4-(trifluoromethyl)phenyl)-5,6-dihydro-1,7-naphthyridine-7(8H)-carboxamide

A solution of (S)-8-(4-(trifluoromethyl)phenyl)-5,6,7,8-tetrahydro-1,7-naphthyridine (60 mg, 0.22 mmol) in DCM (10 mL) was treated with 3-isocyanatopyridine (28 mg, 0.24 mmol) and the mixture was stirred at room temperature for 1 h. The solvent was removed in vacuo and the residue was purified by silica gel chromatography (0-10% MeOH in DCM) to give (S)—N-(pyridin-3-yl)-8-(4-(trifluoromethyl)phenyl)-5,6-dihydro-1,7-naphthyridine-7(8H)-carboxamide as an off-white solid.

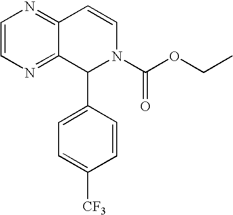

### Example 12

### Ethyl 5-(4-(trifluoromethyl)phenyl)pyrido[3,4-b]pyrazine-6(5H)-carboxylate

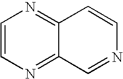

### Step 1. Pyrido[3,4-b]pyrazine

A round-bottomed flask equipped with a reflux condenser was charged with 3,4-diamino pyridine (2.186 g, 20.0 mmol), glyoxal (2.25 mL, 40% aqueous solution, 20.0 mmol), and EtOH (50 mL). The resulting mixture refluxed for 2 h and cooled to room temperature. The solvent was partially removed in vacuo and the residue was triturated with ether (20 mL). The resulting precipitate was collected by filtration to provide pyrido[3,4-b]pyrazine as a tan solid. ¹H NMR (400 MHz, DMSO-d₆): δ 9.52 (s, 1H), 9.20 (d, J= 1.8 Hz, 1H), 9.11 (d, J= 1.6 Hz, 1H), 8.87 (d, J= 5.7 Hz, 1H), 8.05 (d, J= 5.8 Hz, 1H).

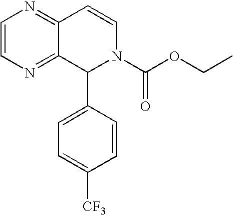

### Step 2. Ethyl 5-(4-(trifluoromethyl)phenyl)pyrido[3,4-b]pyrazine-6(5H)-carboxylate

Analogous to the procedure described for Example 1, treatment of pyrido[3,4-b]pyrazine (949 mg, 7.2 mmol) with ethyl chloformate and (4-(trifluoromethyl)-phenyl)magnesium bromide provided ethyl 5-(4-(trifluoromethyl)phenyl)-pyrido[3,4-b]pyrazine-6(5H)-carboxylate as a yellow oil. MS (ESI pos. ion) m/z: 350 (M+1). ¹H NMR (400 MHz, DMSO-d₆): δ 8.42 (d, J= 2.7 Hz, 1H), 8.35 (d, J= 2.5 Hz, 1H), 7.70 (d, J= 8.2 Hz, 2H), 7.51-7.57 (m, 3H), 6.51 (s, 1H), 6.01 (d, J= 8.0 Hz, 2H), 4.19 (br s, 2H), 1.20-1.25 (m, 2H).

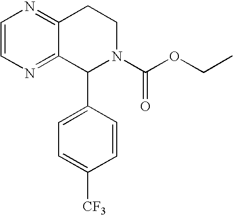

### Example 13

### Ethyl 5-(4-(trifluoromethyl)phenyl)-7,8-dihydropyrido[3,4-b]pyrazine-6(5H)-carboxylate

A solution of ethyl 8-(4-(trifluoromethyl)phenyl)-1,7-naphthyridine-7(8H)-carboxylate (1.65 g, 4.8 mmol) in EtOH (20 mL) was stirred with 10% Pd/C (0.5 g, 4.8 mmol) under hydrogen atmosphere at room temperature for 5 h. The reaction mixture was filtered through a celite pad and the filtrate was concentrated in vacuo. The resulting residue was purified by silica gel chromatography (3% MeOH in DCM) to give ethyl 5-(4-(trifluoromethyl)phenyl)-7,8-dihydropyrido[3,4-b]pyrazine-6(5H)-carboxylate as a yellow oil. MS (ESI pos. ion) m/z: 352 (M+1).

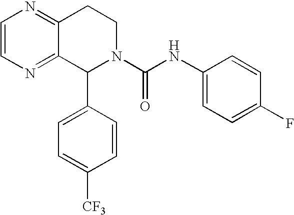

### Example 14

### N-(4-Fluorophenyl)-5-(4-(trifluoromethyl)phenyl)-7,8-dihydropyrido[3,4-b]pyrazine-6(5H)-carboxamide

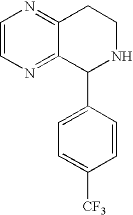

### Step 1. 5-(4-(Trifluoromethyl)phenyl)-5,6,7,8-tetrahydropyrido[3,4-b]pyrazine

A 250-mL, round-bottomed flask was charged with potassium hydroxide (10.0 g, 182 mmol), EtOH (70 mL), the suspension was heated to 80° C. After the potassium hydroxide was dissolved, ethyl 5-(4-(trifluoromethyl)phenyl)-7,8-dihydropyrido[3,4-b]pyrazine-6(5H)-carboxylate (1.28 g, 3.6 mmol) was added and the solution was heated at 90° C. for 3 h. The mixture was allowed to cool to room temperature. The solvent was partially removed in vacuo and the residue was diluted with EtOAc. The organic phase was washed with water, brine, dried over MgSO₄, filtered, and concentrated in vacuo to provide 5-(4-(trifluoro-methyl)phenyl)-5,6,7,8-tetrahydropyrido[3,4-b]pyrazine as a yellow oil. The crude product was used for the next step. MS (ESI pos. ion) m/z: 280 (M+1).

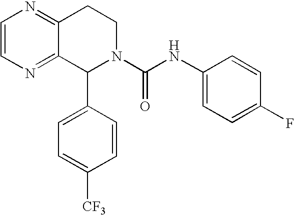

### Step 2. N-(4-Fluorophenyl)-5-(4-(trifluoromethyl)phenyl)-7,8-dihydropyrido[3,4-b]pyrazine-6(5H)-carboxamide

A solution of crude 5-(4-(trifluoromethyl)phenyl)-5,6,7,8-tetrahydropyrido[3,4-b]pyrazine (920 mg, 3.3 mmol) in 1,2-dichloroethane (5 mL) was treated with 4-fluorophenyl isocyanate (0.2 mL, 1.6 mmol) and the mixture was stirred at room temperature for 1 h. The solvent was removed in vacuo and the residue was purified by silica gel chromatography (2-3% MeOH in DCM) to give N-(4-fluoro phenyl)-5-(4-(trifluoromethyl)phenyl)-7,8-dihydropyrido[3,4-b]pyrazine-6(5H)-carboxamide (264 mg, 19% for two steps) as a white solid. MS (ESI pos. ion) m/z: 417 (M+1). ¹H NMR (400 MHz, DMSO-</6): δ 8.91 (s, 1H), 8.58 (dd, J=14.67 Hz, 2.5 Hz, 2H), 7.72 (d, J= 8.2 Hz, 1H), 7.43-7.51 (m, 3H), 7.07-7.11 (m, 2H), 6.66 (s, 1H), 4.30-4.35 (m, 1H), 3.34-3.42 (m, 1H), 3.18-3.25 (m, 1H), 2.98-3.04 (m, 1H).

Purification of racemic N-(4-fluorophenyl)-5-(4-(trifluoromethyl)phenyl)-7,8-dihydropyrido[3,4-b]pyrazine-6(5H)-carboxamide by chiral SFC using the following conditions (Chiralcel OJ-H (250×21 mm), 20% methanol/CO₂ (100 bar), 65 mL/min) provided:

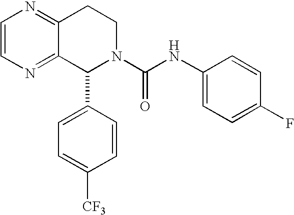

### Example 15

(R)—N-(4-Fluorophenyl)-5-(4-(trifluoromethyl)phenyl)-7,8-dihydropyrido[3,4-b]pyrazine-6(5H)-carboxamide as a white solid (retention time=1.8 min, Chiralcel OJ-H (250×4.6 mm), 20% methanol/CO₂ (100 bar), 4.0 mL/min), MS (ESI pos. ion) m/z: 417 (M+1).

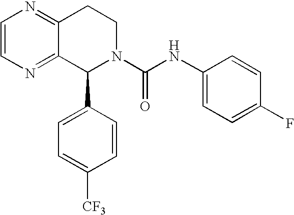

### Example 16

(S)—N-(4-Fluorophenyl)-5-(4-(trifluoromethyl)phenyl)-7,8-dihydropyrido[3,4-b]pyrazine-6(5H)-carboxamide as a white solid (retention time=1.2 min, Chiralcel OJ-H (250×4.6 mm), 20% methanol/CO₂ (100 bar), 4.0 mL/min), MS (ESI pos. ion) m/z: 417 (M+1).

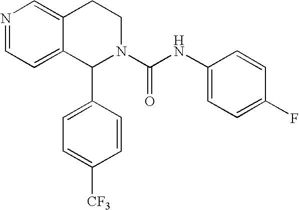

### Example 17

### N-(4-Fluorophenyl)-1-(4-(trifluoromethyl)phenyl)-3,4-dihydro-2,6-naphthyridine-2(1H)-carboxamide

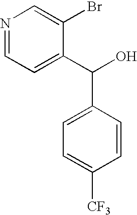

### Step 1. (3-Bromopyridin-4-yl)-(4-(trifluoromethyl)phenyl)methanol

A three-necked 250-mL, round-bottomed flask equipped with a condenser was charged with magnesium (0.92 g, 37.8 mmol), 1-bromo-4-(trifluoromethyl)-benzene (5.3 mL, 37.9 mmol) in THF (35 mL), and the suspension was stirred under nitrogen. Catalytic amount of iodine was added, the mixture was refluxed for 1.5 h, and allowed to cool to room temperature. The reaction mixture was treated with 3-bromoisonicotinaldehyde (3.5 g, 18.9 mmol) and stirred at room temperature for 2 h. The mixture was quenched with saturated NH₄Cl and extracted with EtOAc. The organic layer was washed with water, brine, dried over MgSO₄, filtered, and concentrated in vacuo. The residue was triturated with DCM and the pure product was collected by filtration to give (3-bromopyridin-4-yl)-(4-(trifluoromethyl)phenyl)methanol (5.55 g) as an ivory colored solid. The filtrate was concentrated in vacuo and purified by silica gel chromatography (0-100% EtOAc in hexanes) to give 0.37 g of additional product. ¹H NMR (400 MHz, CDCl₃): δ 8.66 (s, 1H), 8.56 (d, J= 5.0 Hz, 1H), 7.53-7.64 (m, 5H), 6.17 (d, J= 3.7 Hz, 1H), 2.80 (d, J= 3.8 Hz, 1H).

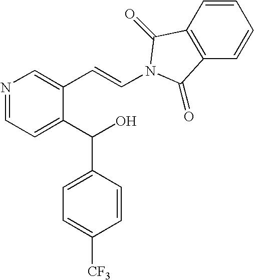

### Step 2. (E)-2-(2-(4-(Hydroxy(4-(trifluoromethyl)phenyl)methyl)pyridin-3-yl)-vinyl)isoindoline-1,3-dione

A 20-mL, microwave reaction vessel was charged with (3-bromopyridin-4-yl)-(4-(trifluoro methyl)phenyl)methanol (2.0 g, 6.02 mmol), 2-vinylisoindoline-1,3-dione (1.16 g, 6.68 mmol), 2-(dicyclohexylphosphino)biphenyl (0.211 g, 0.60 mmol), Pd(dba)₂ (0.176 g, 0.30 mmol), NEt₃ (1.0 mL, 7.23 mmol), and DMF. The mixture was purged with argon and heated in microwave synthesizer at 150° C. for 1 h. The reaction mixture was partitioned between water and EtOAc. The EtOAc layer was separated and aqueous layer was extracted again with EtOAc. The combined organic layers were washed with saturated NaHCO₃, dried over Na₂SO₄, filtered, and concentrated in vacuo. The brown residue was triturated with DCM, the resulting precipitate was collected by filtration to afford the title compound as an ivory colored solid. ¹H NMR (400 MHz, CDCl₃): δ 8.70 (s, 1H), 8.58 (d, J= 5.1 Hz, 1H), 7.92-7.95 (m, 2H), 7.79-7.7.84 (m, 3H), 7.55-7.64 (m, 5H), 7.24 (s, 0.5H), 7.19 (s, 0.5H), 2.71 (d, J= 3.4 Hz, 1H).

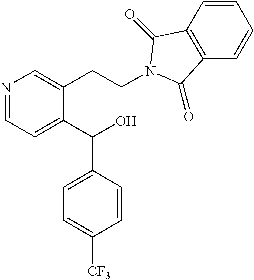

### Step 3. 2-(2-(4-(Hydroxy(4-(trifluoromethyl)phenyl)methyl)pyridin-3-yl)-ethyl) isoindoline-1,3-dione

A 250-mL, round-bottomed flask containing a solution of (E)-2-(2-(4-(hydroxy(4-(trifluoromethyl)phenyl)methyl)pyridin-3-yl)vinyl)isoindoline-1,3-dione (1.0 g, 2.4 mmol) in EtOAc (20 mL) was stirred with 10% Pd on carbon (0.41 g, 3.9 mmol) under 1 atmosphere H₂ at room temperature for 12 h. The catalyst was removed via filtration through a celite pad. The filtrate was concentrated in vacuo to yield the title compounds a yellow solid. The crude product was used for the next step.

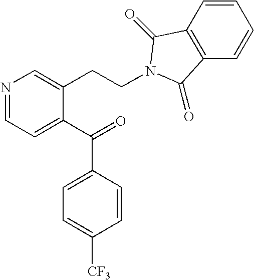

### Step 4. 2-(2-(4-(4-(Trifluoromethyl)benzoyl)pyridin-3-yl)ethyl)isoindoline-1,3-dione

A 150-mL, round-bottomed flask was charged with 2-(2-(4-(hydroxy(4-(trifluoromethyl)phenyl)methyl)pyridin-3-yl)ethyl)isoindoline-1,3-dione (0.92 g, 2.17 mmol) and MnO₂ (7.5 g, 86.41 mmol) in DCM (20 mL) and the mixture was stirred at room temperature for 12 h. MnO₂ was removed via filtration through a celite pad. The filtrate was concentrated in vacuo to afford the title compound as a white solid. ¹H NMR (400 MHz, CDCl₃): δ 8.62 (d, J= 5.0 Hz, 1H), 8.59 (s, 1H), 7.98 (d, J= 8.2 Hz, 2H), 7.66-7.79 (m, 6H), 7.19 (d, J= 4.5 Hz, 1H), 3.91 (t, J= 6.7 Hz, 2H), 3.15 (t, J=6.7 Hz, 2H).

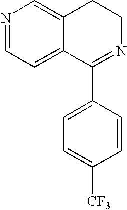

### Step 5. 1-(4-(Trifluoromethyl)phenyl)-3,4-dihydro-2,6-naphthyridine

A 250-mL, round-bottomed flask was charged with 2-(2-(4-(4-(trifluoromethyl)-benzoyl)pyridin-3-yl)ethyl)isoindoline-1,3-dione (1.03 g, 2.42 mmol) and hydrazine hydrate (0.3 mL, 9.68 mmol) in EtOH (50 mL). The reaction mixture was stirred at room temperature for 12 h. The suspension was filtered and the filtrate was concentrated in vacuo. The resulting residue was purified by silica gel chromatography (50-100% EtOAc in hexanes) to give the title compound as a pale yellow oil. MS (ESI pos. ion) m/z: 277 (M+1).

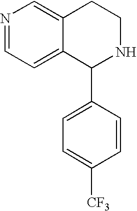

### Step 6. 1-(4-(Trifluoromethyl)phenyl)-1,2,3,4-tetrahydro-2,6-naphthyridine

A solution of 1-(4-(trifluoromethyl)phenyl)-3,4-dihydro-2,6-naphthyridine (36 mg, 0.131 mmol) in MeOH (2.5 mL) was treated with sodium borohydride (17 mg, 0.447 mmol) and the reaction mixture was stirred at room temperature for 30 minutes. MeOH was removed in vacuo and the residue was partitioned between EtOAc and water. The EtOAc layer was separated and the aqueous layer was extracted with EtOAc. The combined organic layers were dried over sodium sulfate, filtered, and concentrated in vacuo to give the title compound (30 mg) as clear oil. The crude product was used for the next step. MS (ESI pos. ion) m/z: 279 (M+1).

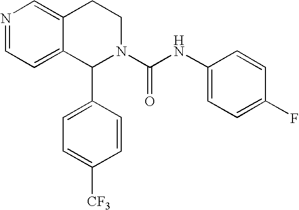

### Step 7. N-(4-Fluorophenyl)-1-(4-(trifluoromethyl)phenyl)-3,4-dihydro-2,6-naphthyridine-2(1H)-carboxamide

A solution of 1-(4-(trifluoromethyl)phenyl)-1,2,3,4-tetrahydro-2,6-naphthyridine (30 mg, 0.1 mmol) in 1,2-dichloroethane (2 mL) was treated with 4-fluorophenyl isocyanate (0.012 mL, 0.11 mmol) and the mixture was stirred at room temperature for 30 minutes. The solvent was removed in vacuo and the residue was purified by silica gel chromatography (0-100% EtOAc in hexanes) to give the title compound as a white solid. MS (ESI pos. ion) m/z: 416. ¹H NMR (400 MHz, CDCl₃): δ 8.54 (s, 1H), 8.49 (d, J= 5.1 Hz, 1H), 7.59 (d, J= 8.2 Hz, 2H), 7.40 (d, J= 8.2 Hz, 2H), 7.27-7.33 (m, 2H), 7.08 (d, J= 5.0 Hz, 1H), 6.98-7.04 (m, 2H), 6.68 (s, 1H), 6.46 (s, 1H), 3.78-3.86 (m, 1H), 3.56-3.65 (m, 1H), 3.03-3.13 (m, 1H), 2.87-2.95 (m, 1H).

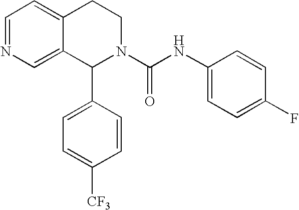

### Example 18

### N-(4-Fluorophenyl)-1-(4-(trifluoromethyl)phenyl)-3,4-dihydro-2,7-naphthyridine-2(1H)-carboxamide

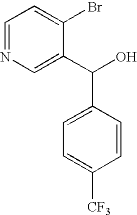

### Step 1. (4-Bromopyridin-3-yl)-(4-(trifluoromethyl)phenyl)methanol

A three-necked 250-mL, round-bottomed flask equipped with a condenser was charged with magnesium (0.27 g, 11.1 mmol) and 1-bromo-4-(trifluoromethyl)-benzene (1.5 mL, 10.9 mmol) in THF (10 mL), and the suspension was stirred under nitrogen. Catalytic amount of iodine was added and the mixture was refluxed for 1.5 h and then allowed to cool to room temperature. The reaction mixture was treated with 4-bromonicotinaldehyde (1.0 g, 5.4 mmol) and stirred at room temperature for 2 h. The mixture was quenched with saturated NH₄Cl and extracted with EtOAc. The organic layer was washed with water, brine, dried over MgSO₄, filtered, and concentrated in vacuo. The residue was triturated with DCM and the pure product was collected by filtration to give (4-bromopyridin-3-yl)(4-(trifluoromethyl)phenyl)methanol (0.76 g) as a tan solid. The filtrate was concentrated in vacuo and purified by silica gel chromatography (30-70% EtOAc in hexanes) to give additional product. ¹H NMR (400 MHz, CDCl₃): δ 8.75 (s, 1H), 8.36 (d, J= 5.3 Hz, 1H), 7.57-7.65 (m, 4H), 7.52 (d, J= 5.3 Hz, 1H), 6.26 (d, J= 3.8 Hz, 1H), 2.65 (d, J= 3.9 Hz, 1H).

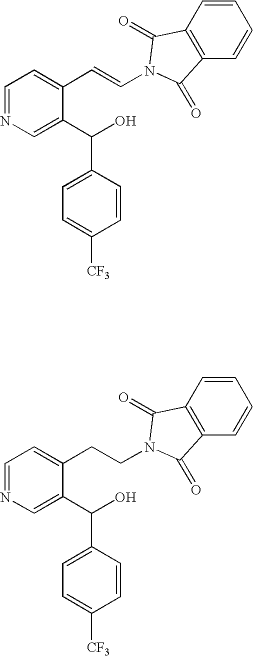

### Step 2. (E)-2-(2-(3-(Hydroxy(4-(trifluoromethyl)phenyl)methyl)pyridin-4-yl)-vinyl)isoindoline-1,3-dione and 2-(2-(3-(hydroxy(4-(trifluoromethyl)phenyl)methyl)pyridin-4-yl)ethyl)isoindoline-1,3-dione

A 20-mL, microwave reaction vessel was charged with (4-bromopyridin-3-yl)-(4-(trifluoro methyl)phenyl)methanol (0.87 g, 2.62 mmol), 2-vinylisoindoline-1,3-dione (499 mg, 2.88 mmol), Pd(dba)₂ (75.3 g, 0.13 mmol), 2-(dicyclohexyl-phosphino)biphenyl (91.8 mg, 0.26 mmol), NEt₃ (0.44 ml, 3.14 mmol), and DMF (2 mL). The mixture was purged with argon and heated in microwave synthesizer at 150° C. for 1 h. The reaction mixture was partitioned between water and EtOAc. The EtOAc layer was separated and aqueous layer was extracted again with EtOAc. The combined organic layers were washed with saturated NaHCO₃, dried over Na₂SO₄, filtered, and concentrated in vacuo. The resulting residue was purified by silica gel chromatography (0-10% MeOH in DCM) to give the mixture (0.4 g) of (E)-2-(2-(3-(hydroxy(4-(trifluoromethyl)phenyl)methyl)pyridin-4-yl)-vinyl) isoindoline-1,3-dione and 2-(2-(3-(hydroxy(4-(trifluoromethyl)phenyl)-methyl)pyridin-4-yl)ethyl)isoindoline-1,3-dione as a light yellow semi-solid. MS (ESI pos. ion) m/z: 425 and 427.

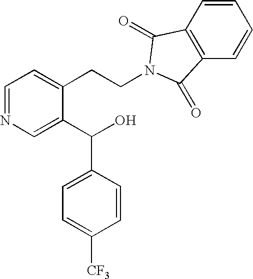

### Step 3. 2-(2-(3-(Hydroxy(4-(trifluoromethyl)phenyl)methyl)pyridin-4-yl)-ethyl)isoindoline-1,3-dione

A 150-mL, round-bottomed flask containing a solution of the product from step 2 in MeOH (50 mL) was stirred with 10% Pd on activated carbon (0.2 g, 1.9 mmol) under 1 atmosphere H₂ at room temperature for 12 h. The catalyst was removed via filtration through a celite pad. The filtrate was concentrated in vacuo to yield the title compound as a gray semi-solid. The crude product was used for the next step.

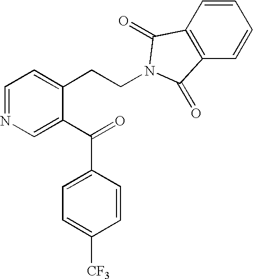

### Step 4. 2-(2-(3-(4-(Trifluoromethyl)benzoyl)pyridin-4-yl)ethyl)isoindoline-1,3-dione

A 100-mL, round-bottomed flask was charged with 2-(2-(3-(hydroxy(4-(trifluoro-methyl)phenyl)methyl)pyridin-4-yl)ethyl)isoindoline-1,3-dione (393 mg, 0.922 mmol) and MnO₂ (2.40 g, 27.7 mmol) in DCM (20 mL) and the mixture was stirred at room temperature for 12 h. MnO₂ was removed via filtration through a celite pad. The filtrate was concentrated in vacuo and purified by silica gel chromatography (0-10% MeOH in DCM) to give the title compound as a white solid. ¹H NMR (400 MHz, CDCl₃): δ 8.62 (d, J= 5.1 Hz, 1H), 8.57 (s, 1H), 7.95 (d, J= 8.0 Hz, 2H), 7.68-7.77 (m, 6H), 7.30 (d, J= 5.1 Hz, 1H), 3.98 (t, J= 6.7 Hz, 2H), 3.24 (t, J= 6.7 Hz, 2H).

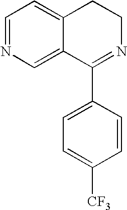

### Step 5. 1-(4-(Trifluoromethyl)phenyl)-3,4-dihydro-2,7-naphthyridine

A 250-mL, round-bottomed flask was charged with 2-(2-(3-(4-(trifluoromethyl)-benzoyl)pyridin-4-yl)ethyl)isoindoline-1,3-dione (130 mg, 0.31 mmol) and hydrazine hydrate (38 uL, 1.2 mmol) in EtOH (50 mL). The reaction mixture was stirred at room temperature for 12 h and concentrated in vacuo. The resulting residue was purified by silica gel chromatography (0-10% MeOH in DCM) to give the title compound as a pale yellow semi-solid. MS (ESI pos. ion) m/z: 277 (M+1).

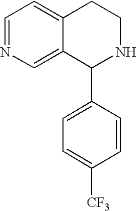

### Step 6. 1-(4-(Trifluoromethyl)phenyl)-1,2,3,4-tetrahydro-2,7-naphthyridine

A solution of 1-(4-(trifluoromethyl)phenyl)-3,4-dihydro-2,7-naphthyridine (32 mg, 0.12 mmol) in MeOH (5 mL) was treated with sodium borohydride (17 mg, 0.447 mmol) and the reaction mixture was stirred at room temperature for 30 minutes. MeOH was removed in vacuo and the residue was partitioned between DCM and water. The DCM layer was separated and the aqueous layer was extracted with DCM. The combined organic layers were dried over sodium sulfate, filtered, and concentrated in vacuo to give the title compound as clear oil. The crude product was used for the next step. MS (ESI pos. ion) m/z: 279 (M+1).

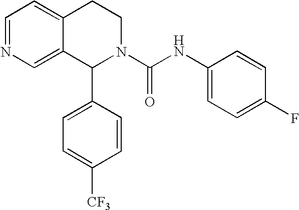

### Step 7. N-(4-Fluorophenyl)-1-(4-(trifluoromethyl)phenyl)-3,4-dihydro-2,7-naphthyridine-2(1H)-carboxamide

A solution of 1-(4-(trifluoromethyl)phenyl)-1,2,3,4-tetrahydro-2,7-naphthyridine (19 mg, 0.068 mmol) in DCM (5 mL) was treated with 4-fluorophenyl isocyanate (0.008 mL, 0.075 mmol) and the mixture was stirred at room temperature for 1 h. The solvent was removed in vacuo and the resulting residue was purified by preparative TLC (0-5% MeOH in DCM) to give the title compound as a yellow solid. MS (ESI pos. ion) m/z: 416 (M+1). ¹H NMR (400 MHz, DMSO-d₆): δ 8.75 (s, 1H), 8.46 (s, 1H), 8.42 (d, J= 5.0 Hz, 1H), 7.71 (d, J= 8.2 Hz, 2H), 7.41-7.51 (m, 4H), 7.32 (d, J= 5.0 Hz, 1H), 7.04-7.13 (m, 2H), 6.72 (s, 1H), 3.97-4.02 (m, 1H), 3.35-3.40 (m, 1H), 2.97-3.05 (m, 1H), 2.79-2.86 (m, 1H).

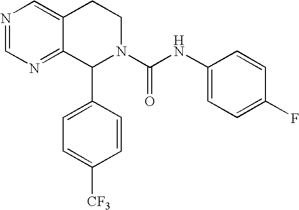

### Example 19

### N-(4-Fluorophenyl)-8-(4-(trifluoromethyl)phenyl)-5,6-dihydropyrido[3,4-d]pyrimidine-7(8H)-carboxamide

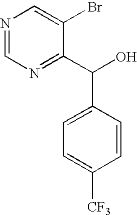

### Step 1. (5-Bromopyrimidin-4-yl)-(4-(trifluoromethyl)phenyl)methanol

A solution of diisopropylamine (2 mL) in anhydrous THF (10 mL) was cooled to −78° C., treated with n-BuLi (2.5M, 5 mL), and stirred at −78° C. A different round-bottomed flask containing a solution of 5-bromopyrimidine (1.01 g, 6.31 mmol) and 4-(trifluoromethyl)benzaldehyde (0.8 mL, 6.31 mmol) in THF (16.5 mL) was cooled to −78° C. The previously made LDA solution (8.5 mL) was added dropwise to this solution. The reaction mixture was stirred for 1.5 h at −78° C. and then for 1 h at 0° C. The reaction was then quenched with ice and extracted with EtOAc. The EtOAc layer was separated, washed with brine, dried over sodium sulfate, filtered, and concentrated in vacuo. The resulting crude product was purified by silica gel chromatography (0-60% EtOAc in hexanes) to give the title compound as a pale yellow oil. ¹H NMR (400 MHz, CDCl₃): δ 9.21 (s, 1H), 8.81 (s, 1H), 7.48-7.62 (m, 4H), 6.01 (d, J= 7.9 Hz, 1H), 4.88 (d, J= 7.9 Hz, 1H).

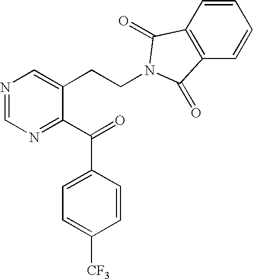

### Step 2. 2-(2-(4-(4-(Trifluoromethyl)benzoyl)pyrimidin-5-yl)ethyl)isoindoline-1,3-dione

A 20-mL, microwave reaction vessel was charged with (5-bromopyrimidin-4-yl)-(4-(trifluoromethyl)phenyl)methanol (560 mg, 1.68 mmol), 2-vinylisoindoline-1,3-dione (327.1 mg, 1.89 mmol), 2-(dicyclohexylphosphino)biphenyl (59.5 mg, 0.17 mmol), Pd(dba)₂ (52.7 mg, 0.092 mmol), NEt₃ (0.3 mL, 2.02 mmol), and DMF (4 mL). The mixture was purged with argon and heated in microwave synthesizer at 150° C. for 1 h. The reaction mixture was partitioned between water and EtOAc. The EtOAc layer was separated and aqueous layer was extracted again with EtOAc. The combined organic layers were washed with saturated NaHCO₃, dried over Na₂SO₄, filtered, and concentrated in vacuo. The resulting residue was purified by silica gel chromatography (0-100% EtOAc in hexanes) to give 2-(2-(4-(4-(trifluoromethyl)benzoyl)pyrimidin-5-yl)ethyl) isoindoline-1,3-dione as a yellow solid. MS (ESI pos. ion) m/z: 426 (M+1). ¹H NMR (400 MHz, CDCl₃): δ 9.20 (s, 1H), 8.79 (s, 1H), 8.03 (d, J= 8.0 Hz, 2H), 7.67-7.74 (m, 6H), 3.98 (t, J= 6.3 Hz, 2H), 3.24 (t, J= 6.5 Hz, 2H). (E)-2-(2-(4-(hydroxy(4-(trifluoromethyl)phenyl)methyl)pyrimidin-5-yl)vinyl)isoindoline-1,3-dione was also collected as a yellow solid. MS (ESI pos. ion) m/z: 424 (M+1).

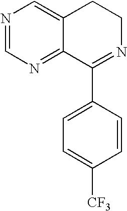

### Step 3. 8-(4-(Trifluoromethyl)phenyl)-5,6-dihydropyrido[3,4-d]pyrimidine

A 25-mL, round-bottomed flask was charged with 2-(2-(4-(4-(trifluoromethyl)-benzoyl)pyrimidin-5-yl)ethyl)isoindoline-1,3-dione (90.7 mg, 0.21 mmol), hydrazine hydrate (0.05 mL, 1.59 mmol) in EtOH (3 mL). The reaction mixture was stirred at room temperature for 12 h. The suspension was filtered through a celite pad and the filtrate was concentrated in vacuo. The resulting residue was purified by silica gel chromatography (30-100% EtOAc in hexanes) to give the title compound as a white solid. ¹H NMR (400 MHz, CDCl₃): δ 9.25 (s, 1H), 8.77 (s, 1H), 7.97 (d, J= 8.0 Hz, 2H), 7.72 (d, J= 8.2 Hz, 2H), 4.07-4.12 (m, 2H), 2.92-2.97 (m, 2H).

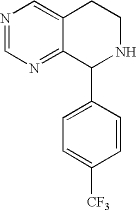

### Step 4. 8-(4-(Trifluoromethyl)phenyl)-5,6,7,8-tetrahydropyrido[3,4-d]pyrimidine

A solution of 8-(4-(trifluoromethyl)phenyl)-5,6-dihydropyrido[3,4-d]pyrimidine (12.4 mg, 0.045 mmol) in MeOH (2 mL) was treated with sodium borohydride (11 mg, 0.24 mmol) and the reaction mixture was stirred at room temperature for 2 h. The solvent was removed in vacuo and the residue was partitioned between EtOAc and water. The EtOAc layer was separated and the aqueous layer was extracted again with EtOAc. The combined organic layers were washed with brine, dried, filtered, and concentrated in vacuo to give the title compound (10.2 mg). The crude product was used for the next step. MS (ESI pos. ion) m/z: 280 (M+1).

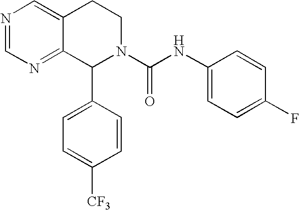

### Step 5. N-(4-Fluorophenyl)-8-(4-(trifluoromethyl)phenyl)-5,6-dihydro-pyrido[3,4-d]pyrimidine-7(8H)-carboxamide

A solution of 8-(4-(trifluoromethyl)phenyl)-5,6,7,8-tetrahydropyrido[3,4-d]pyrimidine (10.2 mg, 0.037 mmol) in 1,2-dichloroethane (5 mL) was treated with 4-fluorophenyl isocyanate (0.007 ml, 0.055 mmol) and the mixture was stirred at room temperature for 1 h. The solvent was removed in vacuo and the resulting residue was purified by silica gel chromatography (0-100% EtOAc in hexanes) and preparative-TLC (EtOAc) to give N-(4-fluorophenyl)-8-(4-(trifluoro methyl)phenyl)-5,6-dihydropyrido[3,4-d]pyrimidine-7(8H)-carboxamide (88 mg, 58% over two steps) as a white solid. MS (ESI pos. ion) m/z: 417 (M+1). ¹H NMR (400 MHz, CDCl₃): δ 9.11 (s, 1H), 8.65 (s, 1H), 7.54-7.65 (m, 4H), 7.23-7.29 (m, 2H), 6.95-7.03 (m, 2H), 6.48 (s, 1H), 6.40 (s, 1H), 4.05-4.12 (m, 1H), 3.56-3.65 (m, 1H), 3.10-3.19 (m, 1H), 2.89-2.97 (m, 1H).

General procedure for prepare examples 20-37: A solution of (R)-8-(4-(trifluoromethyl)phenyl)-5,6,7,8-tetrahydro-1,7-naphthyridine in DCM (3 mL) was treated with the isocyanate and the mixture was stirred at room temperature for 1 h. The solvent was removed in vacuo and the resulting residue was purified by silica gel column chromatography (10-40% EtOAc in hexanes) to provide the corresponding (R)—N-aryl (or alkyl)-5,6-dihydro-1,7-naphthyridine-7(8H)-carboxamide.

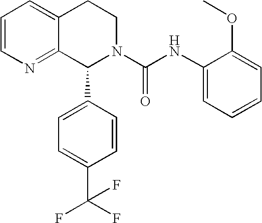

### Example 20

### (R)—N-(2-methoxyphenyl)-8-(4-(trifluoromethyl)phenyl)-5,6-dihydro-1,7-naphthyridine-7(8H)-carboxamide

Obtained as a white solid, MS (ESI pos. ion) m/z: 428 (M+1). ¹H NMR (400 MHz, DMSO-<&): δ 8.46 (dd, J= 4.7 Hz, 1.5 Hz, 1H), 7.90 (s, 1H), 7.71 (d, J=8.3 Hz, 2H), 7.62 (dd, J= 7.8 Hz, 1.2 Hz, 1H), 7.54 (d, J= 8.2 Hz, 2H), 7.33 (dd, J= 7.7 Hz, 4.8 Hz, 1H), 6.98-7.07 (m, 2H), 6.85-6.90 (m, 1H), 6.50 (s, 1H), 3.99-4.07 (m, 1H), 3.78 (s, 1H), 3.45-3.54 (m, 1H), 3.00-3.10 (m, 1H), 2.88 (t, J=4.5 Hz, 0.5H), 2.83 (t, J= 4.6 Hz, 0.5H).

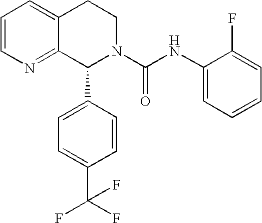

### Example 21

### (R)—N-(2-fluorophenyl)-8-(4-(trifluoromethyl)phenyl)-5,6-dihydro-1,7-naphthyridine-7(8H)-carboxamide

Obtained as a white solid, MS (ESI pos. ion) m/z: 416 (M+1). ¹H NMR (400 MHz, DMSO-d): δ 8.62 (s, 1H), 8.47 (dd, J= 4.7 Hz, 1.3 Hz, 1H), 7.69-7.75 (m, 3H), 7.48 (d, J= 8.3 Hz, 2H), 7.40-7.43 (m, 1H), 7.34 (dd, J= 7.8 Hz, 4.7 Hz, 1H), 7.09-7.24 (m, 3H), 6.55 (s, 1H), 4.07-4.15 (m, 1H), 3.34-3.42 (m, 1H), 3.01-3.11 (m, 1H), 2.80-2.88 (m, 1H).

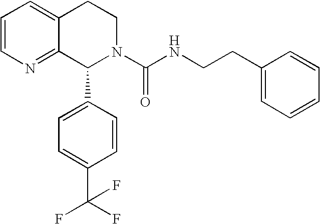

### Example 22

### (R)—N-phenethyl-8-(4-(trifluoromethyl)phenyl)-5,6-dihydro-1,7-naphthyridine-7(8H)-carboxamide

Obtained as an off-white solid, MS (ESI pos. ion) m/z: 426 (M+1). *H NMR (400 MHz, DMSO-d₆): δ 8.45 (d, J= 3.5 Hz, 1H), 7.65-7.68 (m, 3H), 7.38 (d, J=8.2 Hz, 2H), 7.31 (dd, J= 7.7 Hz, 4.8 Hz, 1H), 7.25 (t, J= 7.2 Hz, 2H), 7.18 (t, J= 7.4 Hz, 3H), 6.91 (t, J= 5.3 Hz, 1H), 6.44 (s, 1H), 3.86-3.89 (m, 1H), 3.27-3.37 (m, 2H), 3.13-3.20 (m, 1H), 2.87-2.95 (m, 1H), 2.69-2.76 (m, 3H).

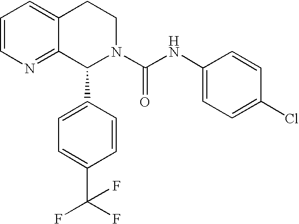

### Example 23

### (R)—N-(4-chlorophenyl)-8-(4-(trifluoromethyl)phenyl)-5,6-dihydro-1,7-naphthyridine-7(8H)-carboxamide

Obtained as a white solid, MS (ESI pos. ion) m/z: 432 (M+1). ¹H NMR (400 MHz, DMSO-d): δ 8.91 (s, 1H), 8.47 (dd, J= 4.7 Hz, 1.4 Hz, 1H), 7.69-7.73 (m, 3H), 7.53-7.56 (m, 2H), 7.46 (d, J= 8.2 Hz, 2H), 7.40-7.43 (m, 1H), 7.28-7.35 (m, 3H), 6.59 (s, 1H), 4.11-4.17 (m, 1H), 3.31-3.38 (m, 1H), 3.02-3.10 (m, 1H), 2.87 (t, J= 3.9 Hz, 0.5H), 2.82 (t, J= 3.8 Hz, 0.5H).

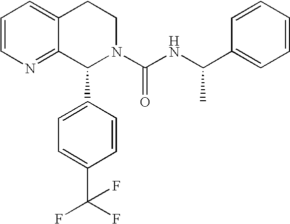

### Example 24

### (R)—N—((S)-1-phenylethyl)-8-(4-(trifluoromethyl)phenyl)-5,6-dihydro-1,7-naphthyridine-7(8H)-carboxamide

Obtained as an off-white solid, MS (ESI pos. ion) m/z: 426 (M+1). ¹H NMR (400 MHz, DMSO-d₆): δ 8.42 (dd, J= 4.7 Hz, 1.7 Hz, 1H), 7.65-7.69 (m, 3H), 7.42 (d, J= 8.3 Hz, 2H), 7.28-7.31 (m, 1H), 7.26 (d, J= 4.4 Hz, 4H), 7.15-20 (m, 1H), 7.11 (d, J= 7.8 Hz, 1H), 6.46 (s, 1H), 4.84-4.92 (m, 1H), 4.03-4.10 (m, 1H), 3.16-3.25 (m, 1H), 2.89-2.99 (m, 1H), 2.73-2.81 (m, 1H), 1.39 (d, J= 7.2 Hz, 3H).

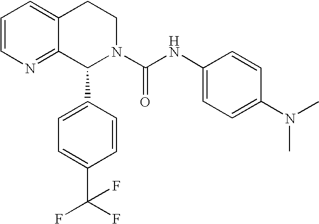

### Example 25

### (R)—N-(4-(dimethylamino)phenyl)-8-(4-(trifluoromethyl)phenyl)-5,6-dihydro-1,7-naphthyridine-7(8H)-carboxamide

Obtained as a tan solid, MS (ESI pos. ion) m/z: 441 (M+1). ¹H NMR (400 MHz, DMSO-6): δ 8.49 (s, 1H), 8.46 (d, J= 4.5 Hz, 1H), 7.70 (t, J= 8.5 Hz, 3H), 7.46 (d, J= 8.0 Hz, 2H), 7.42 (dd, J= 7.5 Hz, 4.5 Hz, 1H), 7.26 (d, J= 8.5 Hz, 2H), 6.66 (d, J= 9.0 Hz, 2H), 6.58 (s, 1H), 4.09-4.12 (m, 1H), 3.27-3.30 (m, 1H), 3.01-3.06 (m, 1H), 2.82 (s, 6H), 2.80-2.82 (m, 1H).

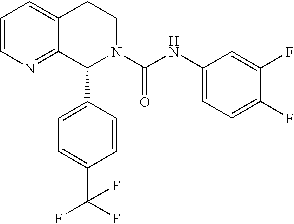

### Example 26

### (R)—N-(3,4-difluorophenyl)-8-(4-(trifluoromethyl)phenyl)-5,6-dihydro-1,7-naphthyridine-7(8H)-carboxamide

Obtained as a white solid, MS (ESI pos. ion) m/z: 434 (M+1). ¹H NMR (400 MHz, DMSO-d₆): δ 8.99 (s, 1H), 8.47 (dd, J= 4.7 Hz, 1.5 Hz 1H), 7.63-7.74 (m, 4H), 7.47 (d, J= 8.2 Hz, 2H), 7.25-7.36 (m, 3H), 6.58 (s, 1H), 4.11-4.12 (m, 1H), 3.30-3.40 (m, 1H), 3.03-3.09 (m, 1H), 2.82-2.89 (m, 1H).

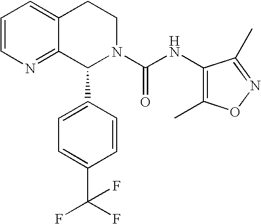

### Example 27

### (R)—N-(3,5-dimethylisoxazol-4-yl)-8-(4-(trifluoromethyl)phenyl)-5,6-dihydro-1,7-naphthyridine-7(8H)-carboxamide

Obtained as an off-white solid, MS (ESI pos. ion) m/z: 417 (M+1). ¹H NMR (400 MHz, DMSO-d₆): δ 8.47 (dd, J= 4.7 Hz, 1.6 Hz, 1H), 8.23 (s, 1H), 7.70-7.73 (m, 3H), 7.34 (dd, J= 7.6 Hz, 4.7 Hz, 1H), 6.50 (s, 1H), 4.03-4.08 (m, 1H), 3.34-3.41 (m, 1H), 3.01-3.09 (m, 1H), 2.85 (t, J= 4.2 Hz, 0.5H), 2.81 (t, J=4.1 Hz, 0.5H), 2.21 (s, 1H), 2.03 (s, 1H).

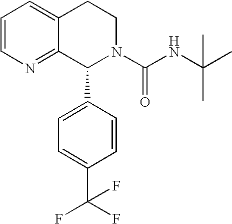

### Example 28

### (R)—N-tert-butyl-8-(4-(trifluoromethyl)phenyl)-5,6-dihydro-1,7-naphthyridine-7(8H)-carboxamide

Obtained as an off-white solid, MS (ESI pos. ion) m/z: 378 (M+1). ¹H NMR (400 MHz, DMSO-d₆): δ 8.43 (dd, J= 4.7 Hz, 1.6 Hz, 1H), 7.68 (d, J= 8.2 Hz, 3H), 7.40 (d, J= 8.2 Hz, 2H), 7.29 (dd, J= 7.7 Hz, 4.8 Hz, 1H), 6.48 (s, 1H), 6.06 (s, 1H), 3.89-3.95 (m, 1H), 3.13-3.19 (m, 1H), 2.89-2.95 (m, 1H), 2.75 (t, J= 3.9 Hz, 0.5H), 2.74 (t, J= 3.9 Hz, 0.5H), 1.27 (s, 9H).

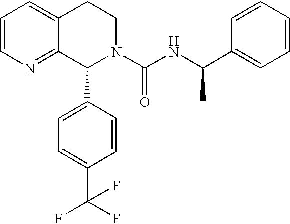

### Example 29

### (R)—N—((R)-1-phenylethyl)-8-(4-(trifluoromethyl)phenyl)-5,6-dihydro-1,7-naphthyridine-7(8H)-carboxamide

Obtained as an off-white solid, MS (ESI pos. ion) m/z: 426 (M+1). ¹H NMR (400 MHz, DMSO-d₆): δ 8.45 (d, J= 4.0 Hz, 1H), 7.64-7.70 (m, 3H), 7.38 (d, J=8.0 Hz, 2H), 7.25-7.34 (m, 5H), 7.19 (t, J= 7.0 Hz, 1H), 7.09 (d, J= 7.5 Hz, 1H), 6.50 (s, 1H), 4.88-4.95 (m, 1H), 4.01-4.04 (m, 1H), 3.20-3.23 (m, 1H), 2.96-2.99 (m, 1H), 2.74-2.78 (m, 1H), 1.39 (d, J= 7.0 Hz, 3H).

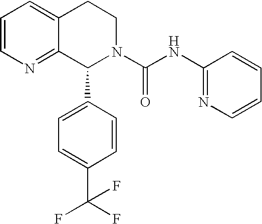

### Example 30

### (R)—N-(pyridin-2-yl)-8-(4-(trifluoromethyl)phenyl)-5,6-dihydro-1,7-naphthyridine-7(8H)-carboxamide

Obtained as a white solid, MS (ESI pos. ion) m/z: 399 (M+1). ¹H NMR (400 MHz, DMSO-d₆): δ 9.42 (s, 1H), 8.46 (dd, J= 4.7 Hz, 1.5 Hz, 1H), 8.26 (dd, J=4.8 Hz, 1.0 Hz, 1H), 7.80-7.83 (m, 1H), 7.67-7.73 (m, 4H), 7.50 (d, J= 8.2 Hz, 2H), 7.33 (dd, J= 1.1 Hz, 4.8 Hz, 1H), 6.98-7.03 (m, 1H), 6.61 (s, 1H), 4.17-4.24 (m, 1H), 3.34-3.43 (m, 1H), 3.02-3.13 (m, 1H), 2.86 (t, J= 3.9 Hz, 0.5H), 2.80 (t, J= 3.9 Hz, 0.5H).

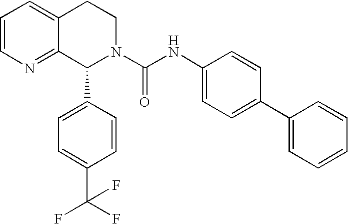

### Example 31

### (R)—N-(4-biphenyl)-8-(4-(trifluoromethyl)phenyl)-5,6-dihydro-1,7-naphthyridine-7(8H)-carboxamide

Obtained as a yellow solid, MS (ESI pos. ion) m/z: 474 (M+1). ¹H NMR (400 MHz, DMSO-d₆): δ 8.90 (s, 1H), 8.47-8.48 (m, 1H), 7.72 (t, J= 7.9 Hz, 3H), 7.56-7.65 (m, 6H), 7.41-7.50 (m, 4H), 7.28-7.36 (m, 2H), 6.63 (s, 1H), 4.16-4.18 (m, 1H), 3.32-3.35 (m, 1H), 3.03-3.11 (m, 1H), 2.83-2.90 (m, 1H).

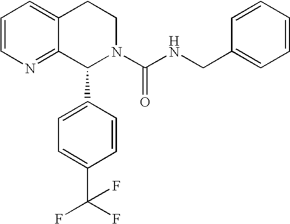

### Example 32

### (R)—N-benzyl-8-(4-(trifluoromethyl)phenyl)-5,6-dihydro-1,7-naphthyridine-7(8H)-carboxamide

Obtained as a pale yellow solid, MS (ESI pos. ion) m/z: 412 (M+1). ¹H NMR (400 MHz, DMSO-d₆): δ 8.45 (dd, J= 4.5 Hz, 1.3 Hz, 1H), 7.66-7.69 (m, 3H), 7.41-7.45 (m, 3H), 7.17-7.32 (m, 6H), 6.47 (s, 1H), 4.23-4.4.37 (m, 2H), 3.93-4.00 (m, 1H), 3.22-3.31 (m, 1H), 2.94-3.06 (m, 1H), 2.74-2.81 (m, 1H).

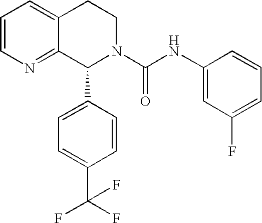

### Example 33

### (R)—N-(3-fluorophenyl)-8-(4-(trifluoromethyl)phenyl)-5,6-dihydro-1,7-naphthyridine-7(8H)-carboxamide

Obtained as a white solid, MS (ESI pos. ion) m/z: 416 (M+1). ¹H NMR (400 MHz, DMSO-d₆): δ 8.98 (s, 1H), 8.47 (dd, J= 4.7 Hz, 1.5 Hz, 1H), 7.71 (t, J=7.8 Hz, 3H), 7.46-7.50 (m, 3H), 7.23-7.35 (m, 3H), 6.75-6.80 (m, 1H), 6.60 (s, 1H), 4.13-4.17 (m, 1H), 3.30-3.40 (m, 1H), 3.03-3.11 (m, 1H), 2.82-2.89 (m, 1H).

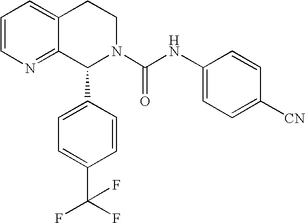

### Example 34

### (R)—N-(4-cyanophenyl)-8-(4-(trifluoromethyl)phenyl)-5,6-dihydro-1,7-naphthyridine-7(8H)-carboxamide

Obtained as a white solid, MS (ESI pos. ion) m/z: 423 (M+1). ¹H NMR (400 MHz, DMSO-d₆): δ 9.24 (s, 1H), 8.48 (dd, J= 4.7 Hz, 1.6 Hz, 1H), 7.69-7.74 (m, 7H), 7.71 (d, J= 8.2 Hz, 2H), 7.34 (dd, J= 1.1 Hz, 4.8 Hz, 1H), 6.60 (s, 1H), 4.14-4.19 (m, 1H), 3.36-3.41 (m, 1H), 3.03-3.11 (m, 1H), 2.89 (t, J= 3.8 Hz, 0.5H), 2.83 (t, J= 3.9 Hz, 0.5H).

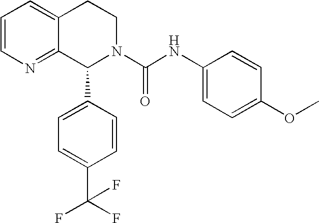

### Example 35

### (R)—N-(4-methoxyphenyl)-8-(4-(trifluoromethyl)phenyl)-5,6-dihydro-1,7-naphthyridine-7(8H)-carboxamide

Obtained as an orange solid, MS (ESI pos. ion) m/z: 428 (M+1). ¹H NMR (400 MHz, DMSO-6): δ 8.63 (s, 1H), 8.47 (dd, J= 4.7 Hz, 1.4 Hz, 1H), 7.70 (t, J=8.5 Hz, 3H), 7.46 (d, J= 8.2 Hz, 2H), 7.36-7.38 (m, 2H), 7.33 (dd, J= 1.1 Hz, 4.8 Hz, 1H), 6.83-6.85 (m, 2H), 6.59 (s, 1H), 4.09-4.15 (m, 1H), 3.28-3.35 (m, 1H), 3.00-3.09 (m, 1H), 2.85 (t, J= 3.8 Hz, 0.5H), 2.81 (t, J= 3.9 Hz, 0.5H).

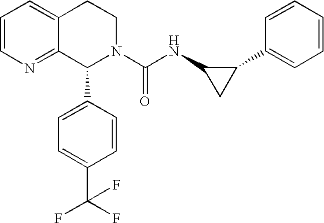

### Example 36

### (R)—N-((1S,2S)-2-phenylcyclopropyl)-8-(4-(trifluoromethyl)phenyl)-5,6-dihydro-1,7-naphthyridine-7(8H)-carboxamide

Obtained as a white solid, MS (ESI pos. ion) m/z: 438 (M+1). ¹H NMR (400 MHz, DMSO-d₆): δ 8.44 (d, J= 3.7 Hz, 1H), 7.68 (d, J= 7.9 Hz, 3H), 7.41 (dd, J= 8.1 Hz, 2.9 Hz, 2H), 7.31 (dd, J= 8.1 Hz, 2.9 Hz, 1H), 7.22-7.27 (m, 2H), 7.09-7.16 (m, 4H), 6.45 (s, 1H), 3.87-3.91 (m, 1H), 3.13-3.22 (m, 1H), 2.93-3.01 (m, 1H), 2.73-2.79 (m, 2H), 1.87-1.98 (m, 1H), 1.17-1.23 (m, 1H), 1.05-1.12 (m, 1H).

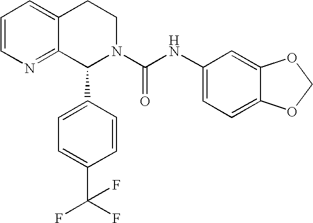

### Example 37

### (R)—N-(benzo[d][1,3]dioxol-5-yl)-8-(4-(trifluoromethyl)phenyl)-5,6-dihydro-1,7-naphthyridine-7(8H)-carboxamide

Obtained as an orange solid, MS (ESI pos. ion) m/z: 442 (M+1). ¹H NMR (400 MHz, DMSO-fife): δ 8.67 (s, 1H), 8.46 (dd, J= 4.7 Hz, 1.4 Hz, 1H), 7.70 (t, J=8.4 Hz, 3H), 7.46 (d, J= 8.2 Hz, 2H), 7.33 (dd, J= 7.8 Hz, 4.7 Hz, 1H), 7.15 (d, J= 2.0 Hz, 1H), 6.79-6.89 (m, 2H), 6.57 (s, 1H), 5.95 (s, 2H), 4.08-4.13 (m, 1H), 3.29-3.35 (m, 1H), 3.02-3.06 (m, 1H), 2.85 (t, J= 3.8 Hz, 0.5H), 2.81 (t, J= 3.8 Hz, 0.5H).

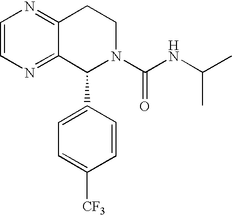

### Example 38

### (R)—N-Isopropyl-5-(4-(trifluoromethyl)phenyl)-7,8-dihydropyrido[3,4-b]pyrazine-6(5H)-carboxamide

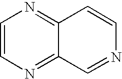

### Step 1. Pyrido[3,4-b]pyrazine

A round-bottomed flask equipped with a reflux condenser was charged with 3,4-diamino pyridine (10.35 g, 94.8 mmol), glyoxal (11.0 mL, 40% aqueous solution, 97.8 mmol), and EtOH (200 mL). The resulting mixture was refluxed for 2 h and cooled to room temperature. The solvent was partially removed in vacuo and the residue was triturated with MTBE (50 mL). The resulting precipitate was collected by filtration to provide pyrido[3,4-b]pyrazine (9.29 g, 70.8 mmol) as a tan solid, m/z calc'd for C₇H₅N₃; 131.1, found 132.0 (M+1). ¹H NMR (300 MHz, DMSO-d₆) δ ppm 9.52 (s, 1H) 9.19 (d, J=1.75 Hz, 1H) 9.11 (d, J=1.61 Hz, 1H) 8.86 (d, J=5.70 Hz, 1H) 8.04 (d, 1H).

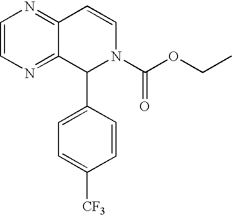

### Step 2. Ethyl 5-(4-(trifluoromethyl)phenyl)pyrido[3,4-b]pyrazine-6(5H)-carboxylate

The 4-trifluoromethylphenyl Grignard reagent was prepared by adding 1-bromo-4-(trifluoromethyl)benzene (10.5 mL, 76.1 mmol) to a suspension of magnesium turnings (1.86 g, 76.5 mmol) and catalytic amount of iodine in THF (66 mL) at room temperature and the mixture was refluxed for 2 h. A different round-bottomed flask containing pyrido[3,4-b]pyrazine (5.02 g, 38.3 mmol) in anhydrous THF (60 mL) was charged with ethyl chloroformate (4.00 mL, 41.8 mmol) under a stream of N2 and the mixture was stirred at room temperature for 20 minutes, and then cooled to 0° C. The previously made Grignard reagent (50 mL, 1.0 M solution) was then added into this solution dropwise and the reaction mixture was stirred for 1 h at 0° C. This mixture was quenched with saturated NH₄Cl and extracted with EtOAc. The organic layer was washed with brine, dried over MgSO₄, filtered, and concentrated in vacuo. The crude product was purified by silica gel chromatography (0-50% EtOAc in hexanes) to give ethyl 5-(4-(trifluoromethyl)phenyl)pyrido[3,4-b]pyrazine-6(5H)-carboxylate (10.7 g, 30.5 mmol) as an orange oil. m/z calc'd for C₁₇H₁₄F₃N₃O₂; 349.1, found 350.0 (M+1). ₁H NMR (300 MHz, CHLOROFORM-d) δ ppm 8.34 (d, J=2.48 Hz, 1H) 8.20-8.30 (m, 1H) 7.34-7.65 (m, 5H) 6.48-6.73 (m, 1H) 6.01 (br. s., 1H) 4.18-4.39 (m, 2H) 1.16-1.45 (m, 3H).

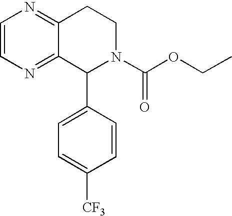

### Step 3. Ethyl 5-(4-(trifluoromethyl)phenyl)-7,8-dihydropyrido[3,4-b]-pyrazine-6(5H)-carboxylate

A solution of ethyl 5-(4-(trifluoromethyl)phenyl)pyrido[3,4-b]pyrazine-6(5H)-carboxylate (10.67 g, 30.5 mmol) and ammonium formate (7.83 g, 63.1 mmol) in EtOH (100 mL) was stirred with 10% Pd/C (1.98 g, 18.6 mmol) at 75° C. for 1 h. The reaction mixture was filtered through a celite pad and the filtrate was concentrated in vacuo. The resulting residue was purified by silica gel chromatography (0-80% EtOAc in hexanes) to give ethyl 5-(4-(trifluoromethyl)-phenyl)-7,8-dihydropyrido[3,4-b]pyrazine-6(5H)-carboxylate (8.96 g, 25.5 mmol) as a clear oil. m/z calc'd for C₁₇H₁₆F₃N₃O₂; 351.1, found 352.0 (M+1). ¹H NMR (300 MHz, CHLOROFORM-d) δ ppm 8.43-8.54 (m, 2H) 7.59 (d, J=8.18 Hz, 2H) 7.39 (d, J=8.18 Hz, 2H) 6.54 (br. s., 1H) 4.18-4.53 (m, 3H) 3.17-3.40 (m, 2H) 2.96-3.14 (m, 1H) 1.32 (t, 3H).

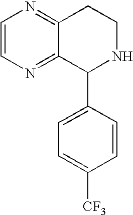

### Step 4. 5-(4-(Trifluoromethyl)phenyl)-5,6,7,8-tetrahydropyrido[3,4-b]-pyrazine

To a stirred solution of ethyl 5-(4-(trifluoromethyl)phenyl)-7,8-dihydropyrido[3,4-b]pyrazine-6(5H)-carboxylate (6.59 g, 18.8 mmol) in 100 mL of chloroform, iodotrimethylsilane (13.3 ml, 93.8 mmol) was added. The dark solution was stirred at 70° C. for 7.5 h then at 65° C. for overnight. The solvent was removed under vacuum after the mixture was cooled down to room temperature. The residue was purified by column chromatography on silica gel (0-10% IP A (w/10% NH₄OH) in CHCl₃) to afford 5-(4-(trifluoromethyl)phenyl)-5,6,7,8-tetra-hydropyrido[3,4-b]pyrazine (3.64 g, 13.0 mmol) as a brown solid, m/z calc'd for C₁₄H₁₂F₃N₃; 279.1, found 280.1 (M+1). ¹H NMR (300 MHz, CHLOROFORM-d) δ ppm 8.50 (d, J=2.05 Hz, 1H) 8.40 (d, J=2.34 Hz, 1H) 7.66 (d, J=8.18 Hz, 2H) 7.50 (d, J=8.04 Hz, 2H) 5.54 (s, 1H) 4.71 (br. s., 1H) 3.40-3.58 (m, 2H) 3.15-3.38 (m, 2H).

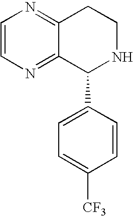

### Step 5. (R)-5-(4-(Trifluoromethyl)phenyl)-5,6,7,8-tetrahydropyrido[3,4-b]pyrazine

Purification of racemic 5-(4-(trifluoromethyl)phenyl)-5,6,7,8-tetrahydro-pyrido[3,4-b]pyrazine (3.80 g, 13.6 mmol) on chiral SFC provided (R)-5-(4-(trifluoromethyl)phenyl)-5,6,7,8-tetrahydropyrido[3,4-b]pyrazine (1.09 g, 3.92 mmol) as a yellow semi-solid. m/z calc'd for C₁₄H₁₂F₃N₃; 279.1, found 280.1 (M+1). ¹H NMR (300 MHz, CHLOROFORM-J) δ ppm 8.38-8.44 (m, 1H) 8.33 (d, J=2.48 Hz, 1H) 7.61 (d, J=8.04 Hz, 2H) 7.43 (d, J=8.18 Hz, 2H) 5.28 (s, 1H) 3.02-3.46 (m, 4H) 2.14 (br. s, 1H).

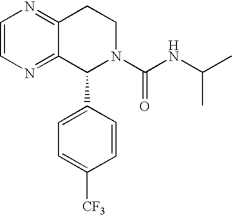

### Step 6. (R)—N-Isopropyl-5-(4-(trifluoromethyl)phenyl)-7,8-dihydropyrido[3,4-b]pyrazine-6(5H)-carboxamide

A solution of (R)-5-(4-(trifluoromethyl)phenyl)-5,6,7,8-tetrahydropyrido[3,4-b]pyrazine (27.3 mg, 0.098 mmol) in DCM (2 mL) was treated with isopropyl isocyanate (0.012 mL, 0.12 mmol) and the mixture was stirred at room temperature for 1 h. The solvent was removed in vacuo and the residue was purified by silica gel chromatography (0-5% IPA (w/10% NH₄OH) in CHCl₃)) to give (R)—N-isopropyl-5-(4-(trifluoromethyl)phenyl)-7,8-dihydropyrido[3,4-b]pyrazine-6(5H)-carboxamide (28.7 mg, 0.079 mmol) as a white solid, m/z calc'd for C₁₈H₁₉F₃N₄O; 364.1, found 365.1 (M+1). ¹H NMR (400 MHz, CHLOROFORM-d) δ ppm 8.48 (s, 2H) 7.59 (d, J=8.22 Hz, 2H) 7.47 (d, J=8.41 Hz, 2H) 6.53 (s, 1H) 4.34 (d, J=7.43 Hz, 1H) 3.88-4.10 (m, 2H) 3.49-3.59 (m, 1H) 3.18-3.30 (m, 1H) 3.05 (dt, J=17.02, 4.21 Hz, 1H) 1.11-1.22 (m, 6H).

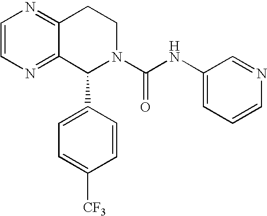

### Example 39

### (R)—N-(Pyridin-3-yl)-5-(4-(trifluoromethyl)phenyl)-7,8-dihydropyrido[3,4-b]pyrazine-6(5H)-carboxamide

A solution of (R)-5-(4-(trifluoromethyl)phenyl)-5,6,7,8-tetrahydropyrido[3,4-b]pyrazine (24.1 mg, 0.086 mmol) in DCM (2 mL) was treated with 3-pyridyl isocyanate (Oakwood, 12.4 mg, 0.10 mmol) and the mixture was stirred at room temperature for 1 h. The solvent was removed in vacuo and the residue was purified by silica gel chromatography (0-5% IPA (w/10% NH₄OH) in CHCl₃)) to give (R)—N-(pyridine-3-yl)-5-(4-(trifluoromethyl)phenyl)-7,8-dihydropyrido[3,4-b]pyrazine-6(5H)-carboxamide (29.2 mg, 0.073 mmol) as a white solid, m/z calc'd for C₂₀H₁₆F₃N₅O; 399.1, found 400.1 (M+1). ¹H NMR (400 MHz, CHLOROFORM-d) δ ppm 8.47-8.55 (m, 2H) 8.41 (d, J=2.54 Hz, 1H) 8.31 (dd, J=4.89, 1.37 Hz, 1H) 7.98 (ddd, J=8.36, 2.59, 1.37 Hz, 1H) 7.62 (d, J=8.22 Hz, 2H) 7.52 (d, J=8.41 Hz, 2H) 7.21-7.30 (m, 1H) 6.66 (s, 1H) 6.59 (s, 1H) 4.08-4.19 (m, 1H) 3.72 (ddd, J=14.08, 10.07, 4.40 Hz, 1H) 3.28-3.40 (m, 1H) 3.11-3.20 (m, 1H).

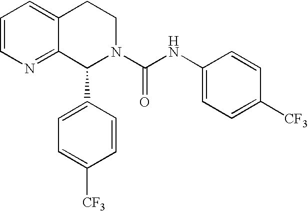

### Example 40

### (R)—N,8-Bis(4-(trifluoromethyl)phenyl)-5,6-dihydro-1,7-naphthyridine-7(8H)-carboxamide

A solution of (R)-8-(4-(trifluoromethyl)phenyl)-5,6,7,8-tetrahydro-1,7-naphthyridine (32.7 mg, 0.12 mmol) in DCM (2 mL) was treated with 1-isocyanato-4-(trifluoromethyl)benzene (0.017 mL, 0.12 mmol) and the mixture was stirred at room temperature for 30 min. The solvent was removed in vacuo and the residue was purified by silica gel chromatography (0-80% EtOAc in hexanes) to afford (R)—N,8-bis(4-(trifluoromethyl)phenyl)-5,6-dihydro-1,7-naphthyridine-7(8H)-carboxamide (51.1 mg, 0.11 mmol) as a white solid, m/z calc'd for C₂₃H₁₇F₆N₃O; 465.1, found 466.1 (M+1). ¹H NMR (300 MHz, CHLOROFORM-J) δ ppm 8.53 (dd, J=4.60, 0.95 Hz, 1H) 7.42-7.65 (m, 9H) 7.24 (dd, J=7.75, 4.82 Hz, 1H) 6.64 (s, 1H) 6.51 (s, 1H) 3.91-4.04 (m, 1H) 3.71 (ddd, J=13.08, 8.62, 4.75 Hz, 1H) 3.09 (br. s., 1H) 2.85-2.99 (m, 1H).

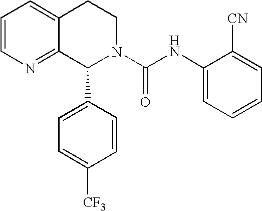

### Example 41

### (R)—N-(2-Cyanophenyl)-8-(4-(trifluoromethyl)phenyl)-5,6-dihydro-1,7-naphthyridine-7(8H)-carboxamide

A solution of (R)-8-(4-(trifluoromethyl)phenyl)-5,6,7,8-tetrahydro-1,7-naphthyridine (30.0 mg, 0.11 mmol) in DCM (2 mL) was treated with 2-isocyanato-benzonitrile (17.5 mg, 0.12 mmol) and the mixture was stirred at room temperature for 30 min. The solvent was removed in vacuo and the residue was purified by silica gel chromatography (30-70% EtOAc in hexanes) to afford (R)—N-(2-cyanophenyl)-8-(4-(trifluoromethyl)phenyl)-5,6-dihydro-1,7-naphthyridine-7(8H)-carboxamide (27.9 mg, 0.066 mmol) as a white solid, m/z calc'd for C₂₃H₁₇F₄N₄O; 422.1, found 423.1 (M+1). ¹H NMR (300 MHz, CHLOROFORM-d) δ ppm 8.54 (dd, J=4.75, 1.53 Hz, 1H) 8.28 (d, J=8.48 Hz, 1H) 7.48-7.64 (m, 7H) 7.20-7.26 (m, 1H) 7.06-7.18 (m, 2H) 6.66 (s, 1H) 3.89-4.01 (m, 1H) 3.77 (ddd, J=13.12, 8.51, 4.82 Hz, 1H) 3.07-3.23 (m, 1H) 2.90-3.03 (m, 1H).

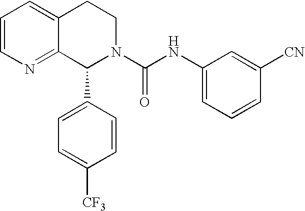

### Example 42

### (R)—N-(3-Cyanophenyl)-8-(4-(trifluoromethyl)phenyl)-5,6-dihydro-1,7-naphthyridine-7(8H)-carboxamide

A solution of (R)-8-(4-(trifluoromethyl)phenyl)-5,6,7,8-tetrahydro-1,7-naphthyridine (30.0 mg, 0.11 mmol) in DCM (2 mL) was treated with 3-cyanophenyl isocyanate (18.2 mg, 0.12 mmol) and the mixture was stirred at room temperature for 30 min. The solvent was removed in vacuo and the residue was purified by silica gel chromatography (0-50% EtOAc in hexanes) to afford (R)—N-(3-cyanophenyl)-8-(4-(trifluoromethyl)phenyl)-5,6-dihydro-1,7-naphthyridine-7(8H)-carboxamide (29.4 mg, 0.070 mmol) as a white solid, m/z calc'd for C₂₃H₁₇F₃N₄O; 422.1, found 423.1 (M+1). ¹H NMR (300 MHz, CHLOROFORM-d) δ ppm 8.53 (dd, J=4.75, 1.53 Hz, 1H) 7.75 (d, J=1.61 Hz, 1H) 7.50-7.64 (m, 6H) 7.30-7.42 (m, 2H) 7.21-7.25 (m, 1H) 6.61 (s, 1H) 6.50 (s, 1H) 3.90-4.03 (m, 1H) 3.71 (ddd, J=13.12, 8.59, 4.75 Hz, 1H) 3.04-3.18 (m, 1H) 2.85-3.00 (m, 1H).

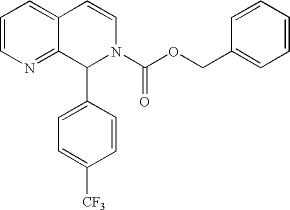

### Example 43

### Benzyl 8-(4-(trifluoromethyl)phenyl)-1,7-naphthyridine-7(8H)-carboxylate

The 4-trifluoromethylphenyl Grignard reagent was prepared analogues to the procedure described in Example 38, step 2, with twice the volume of THF making the concentration 0.5M. To a different round-bottomed flask containing pyrido[3,4-b]pyrazine (0.235 g, 1.81 mmol) in anhydrous THF (6 mL) was added benzyl chloroformate (0.30 mL, 2.02 mmol) dropwise under a stream of N2 and the mixture was stirred at room temperature for 1 h, and more benzyl chloroformate (0.10 mL, 0.67 mmol) was added. After further stirring at room temperature for 15 min, the mixture was cooled to 0° C. The previously made Grignard reagent (5.42 mL, 0.5 M solution) was then added into this solution dropwise and the reaction mixture was stirred for 1.5 h at 0° C. This mixture was quenched with saturated NH₄Cl and extracted with EtOAc. The organic layer was washed with water and brine, dried over MgSO₄, filtered, and concentrated in vacuo. The crude product was purified by silica gel chromatography (0-30% EtOAc in hexanes) to give benzyl 8-(4-(trifluoromethyl)phenyl)-1,7-naphthyridine-7(8H)-carboxylate (0.407 g, 0.99 mmol) as an off-white solid, m/z calc'd for C₂₃H₁₇F₃N₂O₂; 410.1, found 411.1 (M+1). ¹H NMR (300 MHz, CHLOROFORM-d) δ ppm 8.28-8.44 (m, 1H) 7.28-7.60 (m, 10H) 7.06-7.21 (m, 2H) 6.46-6.71 (m, 1H) 5.72-5.91 (m, 1H) 5.13-5.36 (m, 2H).

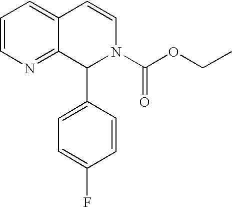

### Example 44

### Ethyl 8-(4-fluorophenyl)-1,7-naphthyridine-7(8H)-carboxylate

A round-bottomed flask containing pyrido[3,4-b]pyrazine (0.31 g, 2.4 mmol) in anhydrous THF (5 mL) was charged with ethyl chloroformate (0.30 mL, 3.1 mmol) under a stream of N2 and the mixture was stirred at room temperature for 10 min, and then cooled to 0° C. 4-Fluorophenylmagnesium bromide (3.1 mL, 1.0M solution) was then added into this solution dropwise and the reaction mixture was stirred for 1.5 h at 0° C. Additional 4-fluorophenylmagnesium bromide (0.5 mL, 1.0M solution) was added dropwise and the reaction mixture was stirred for another 30 min at 0° C. This mixture was quenched with saturated NH₄Cl and extracted with EtOAc. The organic layer was washed with water and brine, dried over MgSO₄, filtered, and concentrated in vacuo. The crude product was purified by silica gel chromatography (10-40% EtOAc in hexanes) to give ethyl 8-(4-fluorophenyl)-1,7-naphthyridine-7(8H)-carboxylate as an yellow oil. m/z calc'd for C₁₇H₁₅FN₂O₂; 298.1, found 299.1 (M+1). ¹H NMR (300 MHz, CHLOROFORM-d) δ ppm 8.35 (br. s., 1H) 7.29-7.42 (m, 3H) 7.00-7.25 (m, 2H) 6.88-7.00 (m, 2H) 6.40-6.64 (m, 1H) 5.80 (dd, J=19.95, 7.82 Hz, 1H) 4.26 (d, J=6.58 Hz, 2H) 1.21-1.40 (m, 3H).

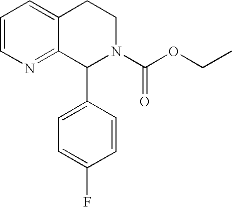

### Example 45

### Ethyl 8-(4-fluorophenyl)-5,6-dihydro-1,7-naphthyridine-7(8H)-carboxylate

Ethyl 8-(4-fluorophenyl)-1,7-naphthyridine-7(8H)-carboxylate (0.585 g, 2.0 mmol) was dissolved in EtOH (10 mL). 10% Pd/C (0.222 g, 2.1 mmol) was added and the flask was evacuated and refilled with hydrogen using balloon. The mixture was stirred at room temperature under balloon pressure of hydrogen for 3.5 h. The catalyst was removed via filtration through a pad pf Celite and concentrated in vacuo to obtain ethyl 8-(4-fluorophenyl)-5,6-dihydro-1,7-naphthyridine-7(8H)-carboxylate (0.578 g, 1.9 mmol) as a clear oil. m/z calc'd for C₁₇H₁₇FN₂O₂; 300.1, found 301.1 (M+1). ¹H NMR (300 MHz, CHLOROFORM-d) δ ppm 8.48 (d, J=3.36 Hz, 1H) 7.53 (d, J=7.75 Hz, 1H) 7.18 (dd, J=7.60, 4.82 Hz, 3H) 6.91-7.04 (m, 2H) 6.45 (br. s., 1H) 4.08-4.36 (m, 3H) 3.15-3.30 (m, 1H) 2.96-3.14 (m, 1H) 2.72-2.87 (m, 1H) 1.31 (t, 3H).

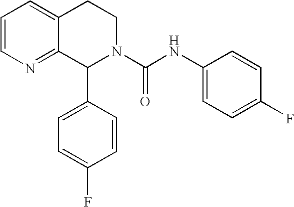

### Example 46

### N,8-Bis(4-fluorophenyl)-5,6-dihydro-1,7-naphthyridine-7(8H)-carboxamide

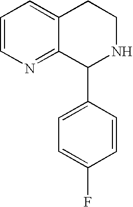

### Step 1. 8-(4-Fluorophenyl)-5,6,7,8-tetrahydro-1,7-naphthyridine

A round-bottomed flask was charged with potassium hydroxide (3.67 g, 65.4 mmol), EtOH (25 mL), and the resulting suspension was heated to 90° C. After the potassium hydroxide was dissolved, ethyl 8-(4-fluorophenyl)-5,6-dihydro-1,7-naphthyridine-7(8H)-carboxylate (0.49 g, 1.6 mmol) and water (2. mL) were added and the solution was refluxed for 10 h. The mixture was allowed to cool to room temperature. The solvent was partially removed in vacuo and the residue was diluted with EtOAc. The organic phase was washed with water, brine, dried over Na₂SO₄, filtered, and concentrated in vacuo. The resulting residue was purified by silica gel chromatography (0-10% IP A (with 10% NH₄OH) in CHCl₃) to afford 8-(4-fluorophenyl)-5,6,7,8-tetrahydro-1,7-naphthyridine (0.216 g, 0.95 mmol) as a yellow oil. m/z calc'd for C₁₄H₁₃FN₂; 228.1, found 229.1 (M+1). ¹H NMR (300 MHz, CHLOROFORM-d) δ ppm 8.38 (dd, J=4.68, 1.46 Hz, 1H) 7.48 (dd, J=7.53, 1.24 Hz, 1H) 7.15-7.24 (m, 2H) 7.10 (dd, J=7.67, 4.75 Hz, 1H) 6.95-7.05 (m, 2H) 5.21 (s, 1H) 3.17-3.31 (m, 1H) 2.96-3.16 (m, 2H) 2.79-2.94 (m, 1H).

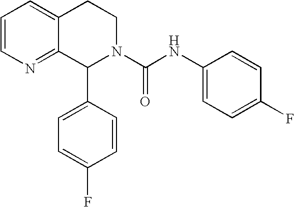

### Step 2N,8-Bis(4-fluorophenyl)-5,6-dihydro-1,7-naphthyridine-7(8H)-carboxamide

A solution of 8-(4-fluorophenyl)-5,6,7,8-tetrahydro-1,7-naphthyridine (35.9 mg, 0.16 mmol) in DCM (3 mL) was treated with 4-fluorophenyl isocyanate (0.025 mL, 0.21 mmol) and the mixture was stirred at room temperature for 1 h. The solvent was removed in vacuo and the residue was purified by silica gel chromatography (30-80% EtOAc in hexanes) followed by washing with ether to give N,8-bis(4-fluorophenyl)-5,6-dihydro-1,7-naphthyridine-7(8H)-carboxamide (40.6 mg, 0.11 mmol) as a white solid, m/z calc'd for C₂₁H₁₇FN₃O; 365.1, found 366.1 (M+1). ¹H NMR (300 MHz, DMSO-d₆) δ ppm 8.79 (s, 1H) 8.44 (dd, J=4.60, 1.39 Hz, 1H) 7.67-7.74 (m, 1H) 7.45-7.55 (m, 2H) 7.31 (dd, J=7.67, 4.75 Hz, 1H) 7.01-7.26 (m, 6H) 6.53 (s, 1H) 4.10 (br. s., 1H) 3.20-3.31 (m, 1H) 2.96-3.11 (m, 1H) 2.76-2.90 (m, 1H).

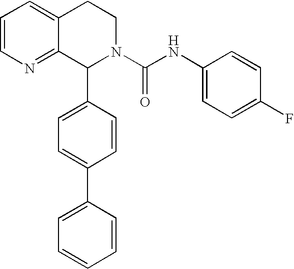

### Example 47

### N-(4-Fluorophenyl)-8-(4-biphenyl)-5,6-dihydro-1,7-naphthyridine-7(8H)-carboxamide

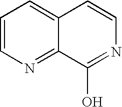

### Step 1. 1,7-Naphthyridin-8-ol

To around-bottomed flask, 1,7-naphthyridin-8-amine (Oakwood, 1.88 g, 13.0 mmol), sulfuric acid (15.0 ml, 281 mmol) and water (3.5 ml, 194 mmol) were mixed. The dark brown mixture was stirred at 215° C. for 18 h. The reaction mixture was cooled to room temperature and poured onto 50 mL of ice. NH₄OH was added slowly to bring the pH to 10 while applying ice bath. The aqueous phase was extracted with CHCl₃ then with 10% iPrOH (w/10% NH₄OH) in CHCl₃. The combined organic phases were dried over Na₂SO₄, filtered and concentrated in vacuo. The crude product was dissolved into a small amount of hot water and upon cooling, the compound crystallized to afford 1,7-naphthyridin-8-ol (1.40 g, 9.55 mmol) as light yellow needles, m/z calc'd for C₈H₆N₂O; 146.1, found 147.0 (M+1). ¹H NMR (300 MHz, DMSO-d₆) δ ppm 11.52 (br. s., 1H) 8.75 (dd, J=4.31, 1.68 Hz, 1H) 8.10 (dd, J=8.04, 1.61 Hz, 1H) 7.67 (dd, J=8.11, 4.31 Hz, 1H) 7.25 (dd, J=6.28, 3.51 Hz, 1H) 6.53 (d, 1H).

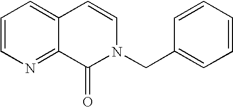

### Step 2. 7-Benzyl-1,7-naphthyridin-8(7H)-one

To a round-bottomed flask, 1,7-naphthyridin-8-ol (0.932 g, 6.4 mmol) and cesium carbonate (2.70 g, 8.30 mmol) were suspended into 10 mL of DMF. Benzyl bromide (0.99 ml, 8.3 mmol) was added and the mixture was stirred at room temperature for 2 h. DMF was removed in vacuo and the residue was partitioned between water and EtOAc. The organic phase was washed with brine, dried over Na₂SO₄, filtered and concentrated in vacuo. The crude product was purified by column chromatography on silica gel (0%-10% IPA (with 10% NH₄OH) in CHCl₃) to afford benzyl-1,7-naphthyridin-8(7H)-one (0.932 g, 3.95 mmol) as a brown oil. m/z calc'd for C₁₅H₁₂N₂O; 236.1, found 237.0 (M+1). ¹H NMR (300 MHz, CHLOROFORM-d) δ ppm 8.90 (dd, J=4.38, 1.61 Hz, 1H) 7.86 (dd, J=8.11, 1.68 Hz, 1H) 7.55 (dd, J=8.04, 4.38 Hz, 1H) 7.29-7.42 (m, 5H) 7.16 (d, J=7.45 Hz, 1H) 6.41 (d, J=7.45 Hz, 1H) 5.31 (s, 2H).

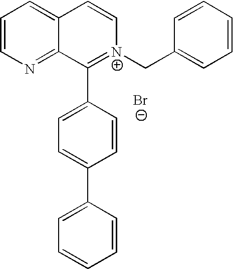

### Step 3. 7-Benzyl-8-(4-biphenyl)-5,6-dihydro-1,7-naphthyridinyl bromide

To a round-bottomed flask, 7-benzyl-1,7-naphthyridin-8(7H)-one (6.00 ml, 1.2 mmol, 0.20 M in THF) and cerium(III) chloride (0.94 g, 3.8 mmol) were mixed. The light brown mixture was stirred at room temperature for 15 min. 4-biphenyl-magnesium bromide (9.5 ml, 4.7 mmol, 0.5 M in toluene) was added and the mixture was stirred at room temperature for 1 h. Water (1.5 mL) was added followed by 16% HBr (6 mL). The brown solution was stirred at room temperature for 30 min. Water was added and the aqueous phase was washed with EtOAc. The aqueous phase was extracted with 10% IPA/CHCl₃. The combined organic phases were dried over Na₂SO₄, filtered and concentrated in vacuo to afford 7-benzyl-8-(4-biphenyl)-5,6-dihydro-1,7-naphthyridinyl bromide (0.38 g, 0.84 mmol) as a yellow foamy solid, m/z calc'd for C₂₇H₂₁N₂⁺; 373.2, found 373.1 (M). ¹H NMR (300 MHz, METHANOL-d) δ ppm 9.25 (dd, J=4.09, 1.75 Hz, 1H) 8.97 (d, 7=6.87 Hz, 1H) 8.77 (dd, J=8.48, 1.61 Hz, 1H) 8.65 (d, J=6.87 Hz, 1H) 8.12 (dd, J=8.48, 4.24 Hz, 1H) 7.86-7.94 (m, 2H) 7.73-7.81 (m, 2H) 7.58-7.66 (m, 2H) 7.49-7.58 (m, 2H) 7.41-7.48 (m, 1H) 7.30-7.40 (m, 3H) 7.08-7.19 (m, 2H) 5.96 (s, 2H).

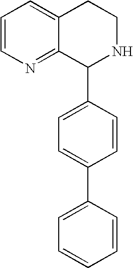

### Step 4. 8-(4-Biphenyl)-5,6,7,8-tetrahydro-1,7-naphthyridine

To a hydrogenation reaction tube, 7-benzyl-8-(4-biphenyl)-5,6-dihydro-1,7-naphthyridinyl bromide (0.38 g, 0.84 mmol) and 10% Pd/C (0.0916 g, 0.86 mmol) were added into 10 mL of EtOH. The tube was evacuated and filled with H₂. The reaction mixture was stirred at room temperature under 45 psi of H₂ for 20 h. Catalyst was removed via filtration through a pad of Celite. The filter cake was washed with methanol. The filtrate was concentrated and the residue was taken up into saturated NaHCO₃ and extracted with EtOAc. The combined organic phases were dried over Na₂SO₄, filtered and concentrated in vacuo. The crude product was purified by silica gel column chromatography (0%-10% IP A (with 10% NH₄OH) in CHCl₃) to afford 8-(4-biphenyl)-5,6,7,8-tetrahydro-1,7-naphthyridine (0.51 g, 0.18 mmol) as a light yellow solid, m/z calc'd for C₂₀H₁₈N₂; 286.2, found 287.1 (M+1). ¹H NMR (300 MHz, CHLOROFORM-d) δ ppm 8.42 (dd, J=4.60, 1.39 Hz, 1H) 7.28-7.62 (m, 10H) 7.12 (dd, J=7.67, 4.75 Hz, 1H) 5.28 (s, 1H) 3.23-3.36 (m, 1H) 2.98-3.19 (m, 2H) 2.83-2.97 (m, 1H).

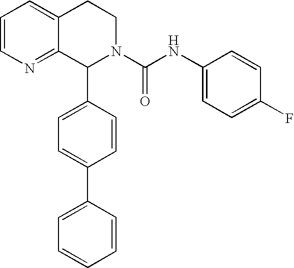

### Step 5. N-(4-Fluorophenyl)-8-(4-biphenyl)-5,6-dihydro-1,7-naphthyridine-7(8H)-carboxamide

To a stirred solution of 8-(4-biphenyl)-5,6,7,8-tetrahydro-1,7-naphthyridine (0.051 g, 0.18 mmol) in 2 mL of DCM, 4-fluorophenyl isocyanate (0.030 ml, 0.26 mmol) was added. The solution was stirred at room temperature for 1.5 h. The reaction mixture was concentrated in vacuo and purified by silica gel column chromatography (0-50% EtOAc in hexanes) to afford N-(4-fluorophenyl)-8-(4-biphenyl)-5,6-dihydro-1,7-naphthyridine-7(8H)-carboxamide (0.068 g, 0.16 mmol) as a white solid, m/z calc'd for C₂₇H₂₂FN₃O; 423.2, found 424.1 (M+1). ¹H NMR (300 MHz, CHLOROFORM-d) δ ppm 8.50 (dd, J=4.75, 1.53 Hz, 1H) 7.47-7.63 (m, 7H) 7.38-7.46 (m, 2H) 7.30-7.38 (m, 1H) 7.15-7.29 (m, 3H) 6.90-7.02 (m, 2H) 6.53 (s, 1H) 6.36 (s, 1H) 3.92-4.06 (m, 1H) 3.73-3.88 (m, 1H) 2.88-3.16 (m, 2H).

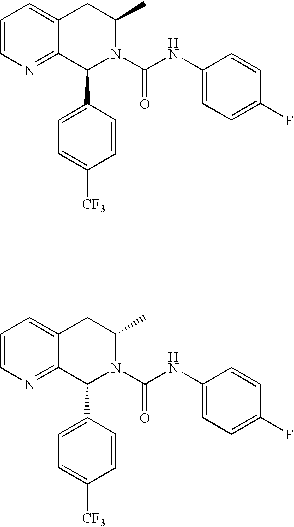

### Example 48

### (6R,8)-N-(4-Fluorophenyl)-6-methyl-8-(4-(trifluoromethyl)phenyl)-5,6-dihydro-1,7-naphthyridine-7(8H)-carboxamide and (6S,8R)—N-(4-fluorophenyl)-6-methyl-8-(4-(trifluoromethyl)phenyl)-5,6-dihydro-1,7-naphthyridine-7(8H)-carboxamide

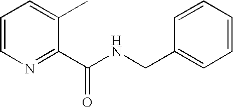

### Step 1. N-Benzyl-3-methylpicolinamide

To a stirred solution of 3-methylpicolinic acid (3.51 g, 25.6 mmol), HBTU (10.7 g, 28.2 mmol) and DIPEA (4.91 ml, 28.2 mmol) in 100 mL of DMF, benzylamine (3.08 ml, 28.2 mmol) was added slowly. The light brown solution was stirred at room temperature for 1.5 h. DMF was evaporated to 30 mL. Aqueous NaHCO₃ was added. The aqueous phase was extracted with EtOAc and the combined organic phases were washed with saturated aqueous NaHCO₃, water, then brine. The organic phase was dried over Na₂SO₄, filtered and concentrated in vacuo. The crude product was purified by column chromatography on silica gel (0-50% EtOAc in hexanes) to afford N-benzyl-3-methylpicolinamide (5.26 g, 23.2 mmol) as a pale yellow oil. m/z calc'd for C₁₄H₁₄N₂O; 226.1, found 227.0 (M+1). ¹H NMR (300 MHz, CHLOROFORM-d) δ ppm 8.50 (br. s., 1H) 8.37 (dd, J=4.60, 1.10 Hz, 1H) 7.60 (dd, J=7.75, 0.88 Hz, 1H) 7.28-7.44 (m, 6H) 4.64 (d, J=6.14 Hz, 2H) 2.79 (s, 3H).

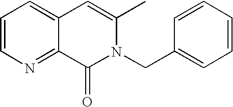

### Step 2. 7-Benzyl-6-methyl-1,7-naphthyridin-8(7H)-one

To a stirred solution of diisopropylamine (5.80 ml, 41.0 mmol) in 20 mL of THF in 150-mL round-bottomed flask, butyllithium (16.4 ml, 41.1 mmol) was added slowly at −12 to −15° C. The mixture was stirred at that temperature for 30 min then cooled to −45 to −50° C. N-Benzyl-3-methylpicolinamide (4.01 g, 17.7 mmol) in a total of 15 mL of THF was added slowly and the mixture was stirred for 30 min. Then methyl acetate (1.48 mL, 18.6 mmol) was added at once. The dark colored mixture was stirred at the temperature for 30 min. The reaction was quenched by adding saturated aqueous NH₄Cl. The aqueous phase was extracted with EtOAc. The combined organic phases were washed with water and brine. The organic phase was dried over Na₂SO₄, filtered and concentrated in vacuo. The crude product was purified by column chromatography on silica gel (30-100% EtOAc in hexanes). The fractions containing the product by LCMS were combined and taken into toluene (20 mL). POCl₃ (3 mL) was added. The light brown solution was heated at 90° C. for 1 h. The reaction mixture was poured into a mixture of ice and saturated NaHCO₃. The aqueous phase was extracted with EtOAc. The combined organic phases were washed with water and brine. The organic phase was dried over Na₂SO₄, filtered and concentrated in vacuo. The crude product was purified by column chromatography on silica gel (0%-5% IPA (with 10% NH₄OH) in CHCl₃) to afford 7-benzyl-6-methyl-1,7-naphthyridin-8(7H)-one (0.203 g, 0.81 mmol) as a brown oil. m/z calc'd for C₁₆H₁₄N₂O; 250.1, found 251.1 (M+1). ¹H NMR (300 MHz, CHLOROFORM-d) δ ppm 8.85 (dd, J=4.31, 1.53 Hz, 1H) 7.79 (dd, J=8.04, 1.61 Hz, 1H) 7.52 (dd, J=8.04, 4.38 Hz, 1H) 7.17-7.33 (m, 5H) 6.29 (s, 1H) 5.49 (s, 2H) 2.35 (s, 3H).

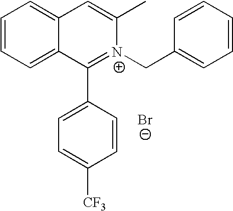

### Step 3. 7-Benzyl-6-methyl-8-(4-trifluoromethylphenyl)-5,6-dihydro-1,7-naphthyridinyl bromide

The 4-trifluoromethylphenyl Grignard reagent was prepared by adding 1-bromo-4-(trifluoromethyl)benzene (0.45 mL, 3.2 mmol) to a suspension of magnesium turnings (0.79 g, 3.2 mmol) and catalytic amount of iodine in THF (10 mL) at room temperature and refluxed for 2 h. To a different round-bottomed flask, 7-benzyl-6-methyl-1,7-naphthyridin-8(7H)-one (0.203 g, 0.81 mmol) and cerium(III) chloride (0.64 g, 2.6 mmol) were mixed into THF (10 mL). The mixture was stirred at room temperature for 30 min. The preformed Grignard reagent was added dropwise and the mixture was stirred at room temperature for 1.5 h. Water (1.5 mL) was added followed by 16% HBr (6 mL). The brown solution was stirred at room temperature for 30 min. Water was added and the aqueous phase was washed with EtOAc. The aqueous phase was extracted with 10% IPA/CHCl₃ then with 25% IPA/CHCl₃ containing 1% NH₄OH. The combined organic phases were dried over Na₂SO₄, filtered and concentrated in vacuo to afford 7-benzyl-6-methyl-8-(4-trifluoromethylphenyl)-5,6-dihydro-1,7-naphthyridinyl bromide (0.21 g, 0.46 mmol) as a light brown oil. m/z calc'd for C₂₃H₁₈F₃N₂⁺; 379.2, found 379.0 (M). ¹H NMR (300 MHz, METHANOL-d₄) δ ppm 9.17 (dd, J=4.02, 1.68 Hz, 1H) 8.67-8.77 (m, 2H) 8.09 (dd, J=8.48, 4.09 Hz, 1H) 7.85 (d, J=8.18 Hz, 2H) 7.69 (d, J=8.18 Hz, 2H) 7.33-7.40 (m, 3H) 6.92-7.00 (m, 2H) 5.94 (s, 2H) 2.96 (s, 3H).

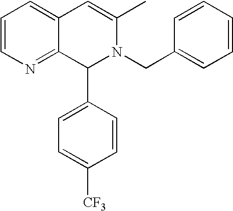

### Step 4. 7-Benzyl-6-methyl-8-(4-(trifluoromethyl)phenyl)-7,8-dihydro-1,7-naphthyridine

To a stirred solution of 7-benzyl-6-methyl-8-(4-trifluoromethylphenyl)-5,6-dihydro-1,7-naphthyridinyl bromide (0.23 g, 0.50 mmol) in MeOH (10 mL), sodium borohydride (0.054 g, 1.4 mmol) was added in potions. After 10 min of stirring at room temperature, most of the methanol was evaporated and the residue was partitioned between water and EtOAc. The aqueous phase was extracted with EtOAc. The combined organic phases were washed with brine. The organic phase was dried over Na₂SO₄, filtered and concentrated in vacuo. The crude product was purified by column chromatography on silica gel (0-30% EtOAc in hexanes) to afford 7-benzyl-6-methyl-8-(4-(trifluoromethyl)phenyl)-7,8-dihydro-1,7-naphthyridine (0.099 g, 0.26 mmol) as a pale yellow oil. m/z calc'd for C₂₃H₁₉F₃N₂; 380.1, found 381.0 (M+1). ¹H NMR (400 MHz, CHLOROFORM-d) δ ppm 8.09 (dd, J=4.69, 1.37 Hz, 1H) 7.53 (s, 4H) 7.18-7.33 (m, 5H) 7.11 (dd, J=7.82, 1.37 Hz, 1H) 6.97 (dd, J=7.73, 4.79 Hz, 1H) 5.66 (s, 1H) 5.14 (s, 1H) 4.80 (d, J=16.43 Hz, 1H) 4.13 (d, J=16.63 Hz, 1H) 2.14 (s, 3H).

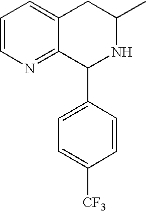

### Step 5. 6-Methyl-8-(4-(trifluoromethyl)phenyl)-5,6,7,8-tetrahydro-1,7-naphthyridine

To a 75 mL pressure tube, 7-benzyl-6-methyl-8-(4-(trifluoromethyl)phenyl)-7,8-dihydro-1,7-naphthyridine (0.099 g, 0.26 mmol) and Pd/C (0.057 g, 0.54 mmol) were mixed into 5 mL of EtOH. HCl (0.50 ml, 2.5 mmol) in isopropanol was added and the mixture was hydrogenated at 48 psi for overnight. The catalyst was removed via filtration through a pad of Celite. The residue was taken into EtOAc and the organic phase was washed with saturated NaHCO₃, water then brine. The organic phase was dried over Na₂SO₄, filtered and concentrated in vacuo to afford 6-methyl-8-(4-(trifluoromethyl)phenyl)-5,6,7,8-tetrahydro-1,7-naphthyridine (0.070 g, 0.24 mmol). This compound was taken to the next step without further

purification, m/z calc'd for C₁₆H₁₅F₃N₂; 292.1, found 293.0 (M+1).

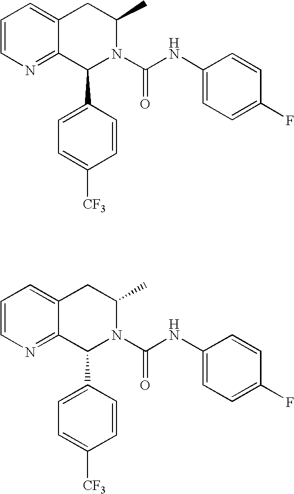

### Step 6. (6R,8S)—N-(4-Fluorophenyl)-6-methyl-8-(4-(trifluoromethyl)phenyl)-5,6-dihydro-1,7-naphthyridine-7(8H)-carboxamide and (6S,8R)—N-(4-fluorophenyl)-6-methyl-8-(4-(trifluoromethyl)phenyl)-5,6-dihydro-1,7-naphthyridine-7(8H)-carboxamide

To a stirred solution of 6-methyl-8-(4-(trifluoromethyl)phenyl)-5,6,7,8-tetrahydro-1,7-naphthyridine (0.070 g, 0.24 mmol) in 3 mL of, 1-fluoro-4-isocyanatobenzene (0.040 ml, 0.35 mmol) was added. The solution was stirred at room temperature for 30 min and concentrated in vacuo. The crude product was purified by column chromatography on silica gel (0-50% EtOAc in hexanes) to afford a mixture of (6R,8S)—N-(4-fluorophenyl)-6-methyl-8-(4-(trifluoromethyl)phenyl)-5,6-dihydro-1,7-naphthyridine-7(8H)-carboxamide and (6R,8R)—N-(4-fluorophenyl)-6-methyl-8-(4-(trifluoromethyl)phenyl)-5,6-dihydro-1,7-naphthyridine-7(8H)-carboxamide (0.080 g, 0.19 mmol) as a white solid. It was confirmed to be cis isomers since an nOe of the methyl protons and aromatic protons on CF₃-phenyl was observed. m/z calc'd for C₂₃H₁₉F₄N₃O; 429.2, found 430.0 (M+1). ¹H NMR (300 MHz, CHLOROFORM-d) δ ppm 8.52 (dd, J=4.97, 1.17 Hz, 1H) 7.49-7.65 (m, 5H) 7.21-7.32 (m, 3H) 6.92-7.03 (m, 2H) 6.48 (s, 1H) 6.34 (s, 1H) 4.34 (ddd, J=9.54, 5.96, 5.85 Hz, 1H) 2.99 (dd, J=15.49, 5.55 Hz, 1H) 2.54 (dd, J=15.49, 9.35 Hz, 1H) 1.47 (d, J=6.28 Hz, 3H).

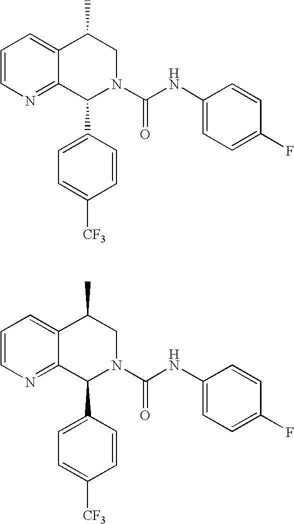

### Example 49

### (5S,8R)—N-(4-Fluorophenyl)-5-methyl-8-(4-(trifluoromethyl)phenyl)-5,6-dihydro-1,7-naphthyridine-7(8H)-carboxamide and (5R,8S)—N-(4-fluorophenyl)-5-methyl-8-(4-(trifluoromethyl)phenyl)-5,6-dihydro-1,7-naphthyridine-7(8H)-carboxamide

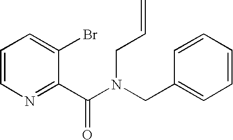

### Step 1. N-Allyl-N-benzyl-3-bromopicolinamide

A 50-mL, round-bottomed flask was charged with 3-bromopicolinic acid (0.73 g, 3.62 mmol), N-benzylprop-2-en-1-amine (0.53 g, 3.62 mmol), HATU (1.38 g, 3.62 mmol), diisopropylethylamine (0.63 mL, 3.62 mmol), and DMF (10 mL). After stirring under a nitrogen atmosphere at room temperature for 4 h, the reaction mixture was diluted with EtOAc. This mixture was washed with saturated NaHCO₃, water, brine, dried over Na₂SO₄, filtered, and concentrated in vacuo. The residue was purified by silica gel chromatography (0-1% MeOH in DCM) to give N-allyl-N-benzyl-3-bromopicolinamide as a light yellow solid. MS (ESI pos. ion) m/z: 331 (M+1).

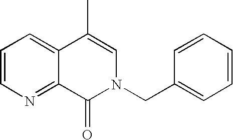

### Step 2. 7-Benzyl-5-methyl-1,7-naphthyridin-8(7H)-one

A 15-mL, reaction vessel was charged with N-allyl-N-benzyl-3-bromopicolinamide (1.04 g, 3.16 mmol), palladium tetrakis triphenyl phosphine (0.18 g, 0.16 mmol), tetrabutylammonium chloride (0.88 g, 3.16 mmol), triethylamine (1.10 mL, 7.90 mmol), and DMF (8 mL). The reaction mixture was heated at 150° C. for 30 min under a nitrogen atmosphere. After cooling to room temperature, it was diluted with EtOAc, washed with water, brine, dried over Na₂SO₄, filtered, and concentrated in vacuo. The residue was purified by silica gel chromatography (0-1% MeOH in DCM) to give the title compound as a dark orange solid. MS (ESI pos. ion) m/z: 251 (M+1).

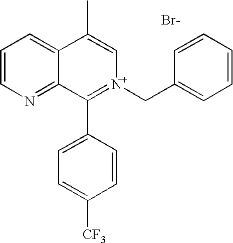

### Step 3. 7-Benzyl-5-methyl-8-(4-(trifluoromethyl)phenyl)-1,7-naphthyridinium bromide

A 50-mL, round-bottomed flask was charged with 7-benzyl-5-methyl-1,7-naphthyridin-8(7H)-one (0.25 g, 1.01 mmol), cerium chloride (0.74 g, 3.02 mmol), and toluene (5 mL). The reaction mixture was stirred at room temperature for 15 min under a nitrogen atmosphere. To this suspension a solution of (4-(trifluoromethyl)phenyl)magnesium bromide, prepared from magnesium turnings (0.048 g, 2.01 mmol) and 4-trifluoromethylbromobenzene (0.28 mL, 2.01 mmol), in THF (4 mL) was added. The reaction mixture was stirred at room temperature for 1 h and then quenched by the addition of 1 mL of water and 0.3 mL of HBr. The reaction mixture was stirred and room temperature for ½ h and then all the solvents were removed under vacuo without any further work up. The residue was purified by flash chromatography (3-5% 2M NH₃/MeOH in DCM) to give the title compound as a dark-yellow solid. MS (ESI pos. ion) m/z: 379 (M+1).

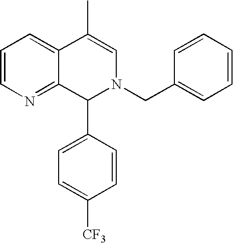

### Step 4. 7-Benzyl-5-methyl-8-(4-(trifluoromethyl)phenyl)-7,8-dihydro-1,7-naphthyridine

To a solution of 7-benzyl-5-methyl-8-(4-(trifluoromethyl)phenyl)-1,7-naphthyridinium bromide (0.66 g, 1.4 mmol) in MeOH (10 mL) was slowly added sodium borohydride (0.22 g, 5.7 mmol). After stirring at room temperature for 1 h, the solvent was partially removed under vacuo. The reaction mixture was diluted with EtOAc, washed with saturated NaHCO₃, water and dried, over Na₂SO₄, filtered, and concentrated in vacuo. The title compounds was obtained as a yellow solid and used without further purification. MS (ESI pos. ion) m/z: 381 (M+1).

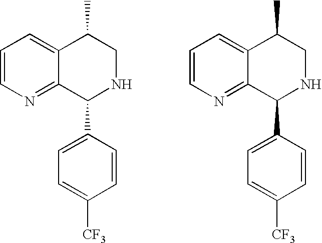

### Step 5. (5S,8R)-5-Methyl-8-(4-(trifluoromethyl)phenyl)-5,6,7,8-tetrahydro-1,7-naphthyridine and (5R,8S)-5-methyl-8-(4-(trifluoromethyl)phenyl)-5,6,7,8-tetrahydro-1,7-naphthyridine

A solution of 7-benzyl-5-methyl-8-(4-(trifluoromethyl)phenyl)-7,8-dihydro-1,7-naphthyridine (0.54 g, 1.42 mmol) in EtOH (10 mL) and hydrogen chloride (4M in dioxane, 1.07 mL, 4.27 mmol) was stirred with 10% palladium on carbon (0.15 g, 1.42 mmol) under 45 psi hydrogen pressure overnight. The reaction mixture was filtered through a Celite pad and the filtrate was concentrated in vacuo. The residue was suspended in EtOAc, washed with saturated NaHCO₃, water, dried over Na₂SO₄, filtered, and concentrated in vacuo. The product was used without further purification. MS (ESI pos. ion) m/z: 293 (M+1).

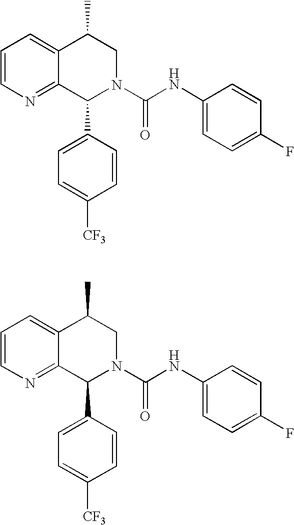

### Step 6. (5S,8R)—N-(4-Fluorophenyl)-5-methyl-8-(4-(trifluoromethyl)phenyl)-5,6-dihydro-1,7-naphthyridine-7(8H)-carboxamide and (5R,8S)—N-(4-fluorophenyl)-5-methyl-8-(4-(trifluoromethyl)phenyl)-5,6-dihydro-1,7-naphthyridine-7(8H)-carboxamide

To a solution of (5S,8R)-5-Methyl-8-(4-(trifluoromethyl)phenyl)-5,6,7,8-tetra-hydro-1,7-naphthyridine and (5R,8S)-5-methyl-8-(4-(trifluoromethyl)phenyl)-5,6,7,8-tetrahydro-1,7-naphthyridine (0.42 g, 1.42 mmol) in DCM (6 mL) at room temperature was added 4-fluorophenyl isocyanate (0.18 mL, 1.57 mmol) and the mixture was stirred at room temperature for 2 h. The solvent was removed under vacuo and the residue was purified first by silica gel flash chromatography (0-1% MeOH in DCM) and then by preparative HPLC (0%-100% MeCN 0.1% TFA/H₂O 0.1% TFA) to give the title compounds as a white solid. MS (ESI pos. ion) m/z: 430 (M+1). ¹H NMR (400 MHz, DMSO-d₆): 8.88 (s, 1H), 8.46 (d, J=4.1 Hz, 1H), 7.93 (d, J= 7.8 Hz, 1H), 7.71 (d, J= 8.2 Hz, 2H), 7.55-7.41 (m, 4H), 7.38 (dd, J= 8.0 Hz, 4.7 Hz, 1H), 7.17-7.01 (m, 2H), 6.61 (s, 1H), 4.24 (dd, J= 14.1 Hz, 5.7 Hz, 1H), 3.24-3.12 (m, 1H), 2.74 (dd, J= 14.2 Hz, 11.6 Hz, 1H), 1.29 (d, J=4.1 Hz, 3H).

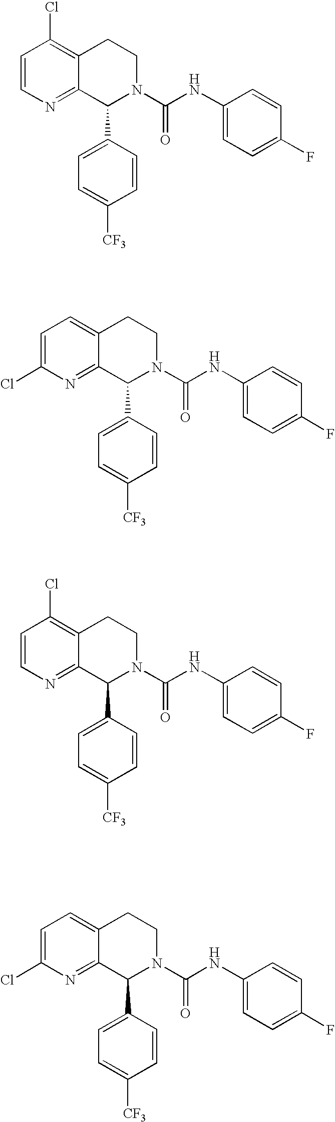

### Examples 50, 51, 52, and 53

### (S)-4-Chloro-N-(4-fluorophenyl)-8-(4-(trifluoromethyl)phenyl)-5,6-dihydro-1,7-naphthyridine-7(8H)-carboxamide, (R)-4-chloro-N-(4-fluorophenyl)-8-(4-(trifluoromethyl)phenyl)-5,6-dihydro-1,7-naphthyridine-7(8H)-carboxamide, (R)-4-chloro-N-(4-fluorophenyl)-8-(4-(trifluoromethyl)phenyl)-5,6-dihydro-1,7-naphthyridine-7(8H)-carboxamide, and (R)-2-chloro-N-(4-fluorophenyl)-8-(4-(trifluoromethyl)phenyl)-5,6-dihydro-1,7-naphthyridine-7(8H)-carboxamide

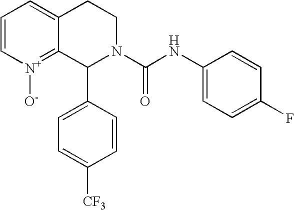

### Step 1. N-(4-Fluorophenyl)-8-(4-(trifluoromethyl)phenyl)-5,6-dihydro-1,7-naphthyridine-7(8H)-carboxamide N-oxide

To a 50-mL round-bottomed flask was added N-(4-fluorophenyl)-8-(4-(trifluoro-methyl)phenyl)-5,6-dihydro-1,7-naphthyridine-7(8H)-carboxamide (714 mg, 1719 μmol), CH₂C₁₂ (2 mL), 3-chloroperoxybenzoic acid (890 mg, 5157 μmol, Aldrich). The reaction mixture was stirred at room temperature for 18 h. The reaction mixture was diluted with 1N NaOH (1 mL) and extracted with EtOAc (2×20 mL). The organic extract was washed with water (2 mL), satd NaCl (2 mL), dried over Na₂SO₄, filtered and concentrated in vacuo to give N-(4-fluorophenyl)-8-(4-(trifluoromethyl)phenyl)-5,6-dihydro-1,7-naphthyridine-7(8H)-carboxamide N-oxide as a crude product. MS (ESI pos. ion) m/z: 432 (M+1).

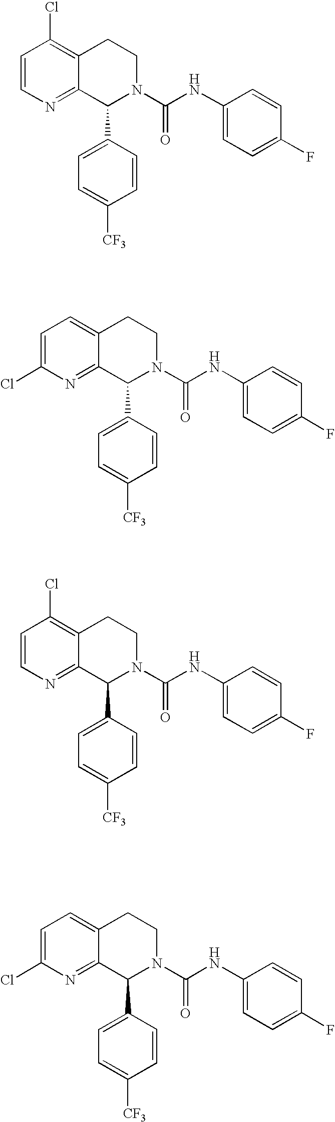

### Step 2. (S)-4-Chloro-N-(4-fluorophenyl)-8-(4-(trifluoromethyl)phenyl)-5,6-dihydro-1,7-naphthyridine-7(8H)-carboxamide, (R)-4-chloro-N-(4-fluorophenyl)-8-(4-(trifluoromethyl)phenyl)-5,6-dihydro-1,7-naphthyridine-7(8H)-carboxamide, (R)-4-chloro-N-(4-fluorophenyl)-8-(4-(trifluoromethyl)phenyl)-5,6-dihydro-1,7-naphthyridine-7(8H)-carboxamide, and (R)-2-chloro-N-(4-fluorophenyl)-8-(4-(trifluoromethyl)phenyl)-5,6-dihydro-1,7-naphthyridine-7(8H)-carboxamide

To a 100-mL, round-bottomed flask was added N-(4-fluorophenyl)-8-(4-(trifluoromethyl)phenyl)-5,6-dihydro-1,7-naphthyridine-7(8H)-carboxamide N-oxide (670 mg, 1553 μmol), phosphorus oxychloride (2172 μl, 23297 μmol, Aldrich). The reaction mixture was stirred at 100° C. for 16 h. The solvent was removed in vacuo and the residue was dissolved in EtOAc (100 mL), washed with sat NaHCO₃ (40 mL), brine (30 mL), dried over Na₂SO₄, filtered and concentrated in vacuo and the residue was purified by silica gel chromatography, eluting with 30% EtOAc/hexanes to give the crude product. The four isomers were separated by chiral HPLC using the following method (Column: Chiralcel OD-H, (20×250 nm, 5 um), Solvent: Methanol (0.2% DeA), Flow rate: 70 mL/min, outlet pressure: 100 bar) to give to give (S)-4-Chloro-N-(4-fluorophenyl)-8-(4-(trifluoro-methyl)phenyl)-5,6-dihydro-1,7-naphthyridine-7(8H)-carboxamide (121 mg, 17% yield). MS (ESI pos. ion) m/z: 450 (M+1). ¹H NMR (300 MHz, CHLOROFORM-d) δ ppm 2.88-3.03 (m, 1H), 3.06-3.22 (m, 1H), 3.50-3.62 (m, 1H), 3.91-4.09 (m, 1H), 6.44 (s, 1H), 6.57 (s, 1H), 6.93-7.04 (m, 2H), 7.27-7.35 (m, 3H), 7.49 (d, J= 5.18 HZ, 2H), 7.59 (d, 2H), 8.42 (d, J=5.12 Hz, 1H); (R)-4-Chloro-N-(4-fluorophenyl)-8-(4-(trifluoromethyl)phenyl)-5,6-dihydro-1,7-naphthyridine-7(8H)-carboxamide (115 mg, 16% yield), MS (ESI pos. ion) m/z: 450 (M+1). ¹H NMR (300 MHz, CHLOROFORM-d) δ ppm 2.88-3.03 (m, 1H), 3.06-3.22 (m, 1H), 3.50-3.62 (m, 1H), 3.91-4.09 (m, 1H), 6.44 (s, 1H), 6.57 (s, 1H), 6.93-7.04 (m, 2H), 7.27-7.35 (m, 3H), 7.49 (d, 2H), 7.59 (d, J= 5.17 Hz, 2H), 8.42 (d, J=5.12 Hz, 1H). (R)-4-Chloro-N-(4-fluorophenyl)-8-(4-(trifluoromethyl)phenyl)-5,6-dihydro-1,7-naphthyridine-7(8H)-carboxamide (115 mg, 16% yield), MS (ESI pos. ion) m/z: 450 (M+1). ¹H NMR (300 MHz, CHLOROFORM-d) δ ppm 2.79-2.93 (m, 1H), 2.99-3.16 (m, 1H), 3.53-3.69 (m, 1H), 3.89-4.04 (m, 1H), 6.36 (s, 1H), 6.45 (s, 1H), 6.94-7.04 (m, 2H), 7.20-7.33 (m, 2H), 7.48-7.56 (m, 4H), 7.57-7.66 (m, 2H); and (R)-2-Chloro-N-(4-fluorophenyl)-8-(4-(trifluoromethyl)phenyl)-5,6-dihydro-1,7-naphthyridine-7(8H)-carboxamide (78 mg, 11% yield). MS (ESI pos. ion) m/z: 450 (M+1). ¹H NMR (300 MHz, CHLOROFORM-d) δ ppm 2.79-2.93 (m, 1H), 2.99-3.16 (m, 1H), 3.53-3.69 (m, 1H), 3.89-4.04 (m, 1H), 6.36 (s, 1H), 6.45 (s, 1H), 6.94-7.04 (m, 2H), 7.20-7.33 (m, 2H), 7.48-7.56 (m, 4H), 7.57-7.66 (m, 2H).

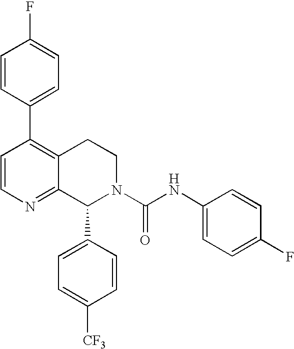

### Example 54

### (R)—N,4-bis(4-fluorophenyl)-8-(4-(trifluoromethyl)phenyl)-5,6-dihydro-1,7-naphthyridine-7(8H)-carboxamide

To a 50-mL, round-bottomed flask was added (R)-4-chloro-N-(4-fluorophenyl)-8-(4-(trifluoromethyl)phenyl)-5,6-dihydro-1,7-naphthyridine-7(8H)-carboxamide (30 mg, 67 μmol), 4-fluorobenzeneboronic acid (11 mg, 80 μmol, Aldrich), tetrakis(triphenylphosphine)palladium (7.7 mg, 6.7 μmol), sodium carbonate (14 mg, 133 μmol), and dioxane (0.5 mL). The reaction mixture was stirred at 90° C. for 18 h. The reaction mixture was diluted with water (5 mL) and extracted with EtOAc (2×20 mL). The organic extract was washed with water (5 mL), satd NaCl (5 mL), dried over Na₂SO₄, filtered and concentrated in vacuo and the residue was purified by silica gel chromatography, eluting with 30% EtOAc/hexanes to give (R)—N,4-bis(4-fluorophenyl)-8-(4-(trifluoromethyl)-phenyl)-5,6-dihydro-1,7-naphthyridine-7(8H)-carboxamide (18 mg, 53% yield) as a white solid. MS (ESI pos. ion) m/z: 510 (M+1). ¹H NMR (300 MHz, CHLOROFORM-d) δ ppm 2.67-2.84 (m, 1H), 2.95-3.15 (m, 1H), 3.74 (t, J=6.14 Hz, 2H), 6.37 (s, 1H), 6.54 (s, 1H), 6.97 (t, J=8.70 Hz, 2H), 7.12-7.22 (m, 3H), 7.21-7.36 (m, 4H), 7.57-7.64 (m, 4H), 8.55 (d, J=4.97 Hz, 1H).

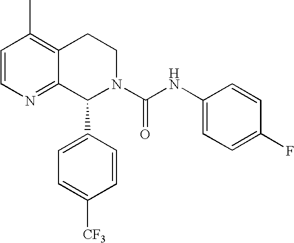

### Example 55

### (R)—N-(4-fluorophenyl)-4-methyl-8-(4-(trifluoromethyl)phenyl)-5,6-dihydro-1,7-naphthyridine-7(8H)-carboxamide

To a 50-mL, round-bottomed flask was added (R)-4-chloro-N-(4-fluorophenyl)-8-(4-(trifluoromethyl)phenyl)-5,6-dihydro-1,7-naphthyridine-7(8H)-carboxamide (21 mg, 47 μmol), methaneboronic acid (3 mg, 56 μmol, Aldrich), tetrakis(triphenylphosphine)palladium (11 mg, 9 μmol), and dioxane (0.5 mL), potassium carbonate (47 μl, 93 μmol). The reaction mixture was stirred at 90° C. for 18 h. The reaction mixture was diluted with water (5 mL) and extracted with EtOAc (2×20 mL). The organic extract was washed with water (5 mL), satd NaCl (5 mL), dried over Na₂SO₄, filtered and concentrated in vacuo and the residue was purified by silica gel chromatography, eluting with 40% EtOAc/hexanes to give (R)—N-(4-fluorophenyl)-4-methyl-8-(4-(trifluoromethyl)-phenyl)-5,6-dihydro-1,7-naphthyridine-7(8H)-carboxamide (11 mg, 55% yield) as a white solid. MS (ESI pos. ion) m/z: 430 (M+1). ¹H NMR (300 MHz, CHLOROFORM-d) δ ppm 2.31 (s, 3H), 2.69-2.82 (m, 1H), 2.94-3.11 (m, 1H), 3.47-3.61 (m, 1H), 3.94-4.07 (m, 1H), 6.48 (d, J=11.98 Hz, 2H), 6.97, (t, J=8.70 Hz, 2H), 7.08 (d, J=4.97 Hz, 1H), 7.26-7.34 (m, 1H), 7.47-7.54 (m, 2H), 7.54-7.63 (m, 2H), 8.37 (d, J=4.82 Hz, 1H).

### Assays

Luminescence readout assay for measuring intracellular calcium. Stable CHO cell lines expressing human TRPM8 were generated using tetracycline inducible T-REx™ expression system from Invitrogen, Inc (Carlsbad, Calif.). In order to enable a luminescence readout based on intracellular increase in calcium (Le Poul et al., 2002), each cell line was also co-transfected with pcDNA3.1 plasmid containing jelly fish aequorin cDNA. Twenty four hours before the assay, cells were seeded in 96-well plates and TRP channel expression was induced with 0.5 μg/ml tetracycline. On the day of the assay, culture media was removed and cells were incubated with assay buffer (F12 containing 30 mM HEPES for TRPM8 containing 15 μM coelenterazine (P.J.K, Germany) for 2 hours. Potential antagonists were added and cells were incubated for 2.5 min prior to adding agonist, 1 μM Icilin, or 1 min prior to addition of cold buffer (<10° C.). The luminescence was measured by a CCD camera based FLASH-luminometer built by Amgen, Inc. Compound activity was calculated using GraphPad Prism 4.01 (GraphPad Software Inc, San Diego, Calif.).

The following compounds exhibit IC₅₀ values of less than 5 μM in the above assay using the cold buffer and human TRPM8:
(5R)—N-(4-Fluorophenyl)-5-(4-(trifluoromethyl)phenyl)-7,8-dihydro-1,6-naphthyridine-6(5H)-carboxamide;
(R)—N-((1S,2S)-2-phenylcyclopropyl)-8-(4-(trifluoromethyl)phenyl)-5,6-dihydro-1,7-naphthyridine-7(8H)-carboxamide;
(R)—N—((R)-1-phenylethyl)-8-(4-(trifluoromethyl)phenyl)-5,6-dihydro-1,7-naphthyridine-7(8H)-carboxamide;
(R)—N—((S)-1-phenylethyl)-8-(4-(trifluoromethyl)phenyl)-5,6-dihydro-1,7-naphthyridine-7(8H)-carboxamide;
(R)—N-(2-fluorophenyl)-8-(4-(trifluoromethyl)phenyl)-5,6-dihydro-1,7-naphthyridine-7(8H)-carboxamide;
(R)—N-(2-methoxyphenyl)-8-(4-(trifluoromethyl)phenyl)-5,6-dihydro-1,7-naphthyridine-7(8H)-carboxamide;
(R)—N-(3,4-difluorophenyl)-8-(4-(trifluoromethyl)phenyl)-5,6-dihydro-1,7-naphthyridine-7(8H)-carboxamide;
(R)—N-(3,5-dimethylisoxazol-4-yl)-8-(4-(trifluoromethyl)phenyl)-5,6-dihydro-1,7-naphthyridine-7(8H)-carboxamide;
(R)—N-(3-fluorophenyl)-8-(4-(trifluoromethyl)phenyl)-5,6-dihydro-1,7-naphthyridine-7(8H)-carboxamide;
(R)—N-(4-(dimethylamino)phenyl)-8-(4-(trifluoromethyl)phenyl)-5,6-dihydro-1,7-naphthyridine-7(8H)-carboxamide;
(R)—N-(4-biphenyl)-8-(4-(trifluoromethyl)phenyl)-5,6-dihydro-1,7-naphthyridine-7(8H)-carboxamide;
(R)—N-(4-chlorophenyl)-8-(4-(trifluoromethyl)phenyl)-5,6-dihydro-1,7-naphthyridine-7(8H)-carboxamide;
(R)—N-(4-cyanophenyl)-8-(4-(trifluoromethyl)phenyl)-5,6-dihydro-1,7-naphthyridine-7(8H)-carboxamide;
(R)—N-(4-Fluorophenyl)-5-(4-(trifluoromethyl)phenyl)-7,8-dihydropyrido[3,4-b]pyrazine-6(5H)-carboxamide;
(R)—N-(4-Fluorophenyl)-8-(4-(trifluoromethyl)phenyl)-5,6-dihydro-1,7-naphthyridine-7(8H)-carboxamide;
(R)—N-(4-methoxyphenyl)-8-(4-(trifluoromethyl)phenyl)-5,6-dihydro-1,7-naphthyridine-7(8H)-carboxamide;
(R)—N-(benzo[d][1,3]dioxol-5-yl)-8-(4-(trifluoromethyl)phenyl)-5,6-dihydro-1,7-naphthyridine-7(8H)-carboxamide;
(R)—N-(pyridin-2-yl)-8-(4-(trifluoromethyl)phenyl)-5,6-dihydro-1,7-naphthyridine-7(8H)-carboxamide;
(R)—N-(pyridin-3-yl)-8-(4-(trifluoromethyl)phenyl)-5,6-dihydro-1,7-naphthyridine-7(8H)-carboxamide;
(R)—N-benzyl-8-(4-(trifluoromethyl)phenyl)-5,6-dihydro-1,7-naphthyridine-7(8H)-carboxamide;
(R)—N-phenethyl-8-(4-(trifluoromethyl)phenyl)-5,6-dihydro-1,7-naphthyridine-7(8H)-carboxamide;
(R)—N-tert-butyl-8-(4-(trifluoromethyl)phenyl)-5,6-dihydro-1,7-naphthyridine-7(8H)-carboxamide;
(S)—N-(4-Fluorophenyl)-5-(4-(trifluoromethyl)phenyl)-7,8-dihydropyrido[3,4-b]pyrazine-6(5H)-carboxamide;
(S)—N-(4-Fluorophenyl)-8-(4-(trifluoromethyl)phenyl)-5,6-dihydro-1,7-naphthyridine-7(8H)-carboxamide;
(S)—N-(pyridin-3-yl)-8-(4-(trifluoromethyl)phenyl)-5,6-dihydro-1,7-naphthyridine-7(8H)-carboxamide;
Ethyl 5-(4-(trifluoromethyl)phenyl)-1,6-naphthyridine-6(5H)-carboxylate;
Ethyl 5-(4-(trifluoromethyl)phenyl)pyrido[3,4-b]pyrazine-6(5H)-carboxylate;
Ethyl 8-(4-(trifluoromethyl)phenyl)-1,7-naphthyridine-7(8H)-carboxylate;
N-(4-Fluorophenyl)-1-(4-(trifluoromethyl)phenyl)-3,4-dihydro-2,7-naphthyridine-2(1H)-carboxamide;
N-(4-Fluorophenyl)-1-(4-(trifluoromethyl)phenyl)-3,4-dihydro-2,6-naphthyridine-2(1H)-carboxamide;
N-(4-Fluorophenyl)-5-(4-(trifluoromethyl)phenyl)-7,8-dihydro-1,6-naphthyridine-6(5H)-carboxamide;
N-(4-Fluorophenyl)-5-(4-(trifluoromethyl)phenyl)-7,8-dihydropyrido[3,4-b]pyrazine-6(5H)-carboxamide;
N-(4-Fluorophenyl)-8-(4-(trifluoromethyl)phenyl)-5,6-dihydro-1,7-naphthyridine-7(8H)-carboxamide;
N-(4-Fluorophenyl)-8-(4-(trifluoromethyl)phenyl)-5,6-dihydropyrido[3,4-d]pyrimidine-7(8H)-carboxamide;
(R)—N-Isopropyl-5-(4-(trifluoromethyl)phenyl)-7,8-dihydropyrido[3,4-b]pyrazine-6(5H)-carboxamide;
(R)—N-(Pyridin-3-yl)-5-(4-(trifluoromethyl)phenyl)-7,8-dihydropyrido[3,4-b]pyrazine-6(5H)-carboxamide;
(R)—N,8-Bis(4-(trifluoromethyl)phenyl)-5,6-dihydro-1,7-naphthyridine-7(8H)-carboxamide;
(R)—N-(2-Cyanophenyl)-8-(4-(trifluoromethyl)phenyl)-5,6-dihydro-1,7-naphthyridine-7(8H)-carboxamide;
(R)—N-(3-Cyanophenyl)-8-(4-(trifluoromethyl)phenyl)-5,6-dihydro-1,7-naphthyridine-7(8H)-carboxamide;
Benzyl 8-(4-(trifluoromethyl)phenyl)-1,7-naphthyridine-7(8H)-carboxylate;
Ethyl 8-(4-fluorophenyl)-1,7-naphthyridine-7(8H)-carboxylate;
Ethyl 8-(4-fluorophenyl)-5,6-dihydro-1,7-naphthyridine-7(8H)-carboxylate;
N,8-Bis(4-fluorophenyl)-5,6-dihydro-1,7-naphthyridine-7(8H)-carboxamide;
N-(4-Fluorophenyl)-8-(4-biphenyl)-5,6-dihydro-1,7-naphthyridine-7(8H)-carboxamide;
(6,8)-N-(4-Fluorophenyl)-6-methyl-8-(4-(trifluoromethyl)phenyl)-5,6-dihydro-1,7-naphthyridine-7(8H)-carboxamide (racemic);
(5,8)-N-(4-Fluorophenyl)-5-methyl-8-(4-(trifluoromethyl)phenyl)-5,6-dihydro-1,7-naphthyridine-7(8#)-carboxamide (racemic);
(R)—N,4-bis(4-fluorophenyl)-8-(4-(trifluoromethyl)phenyl)-5,6-dihydro-1,7-naphthyridine-7(8H)-carboxamide; and
(R)—N-(4-fluorophenyl)-4-methyl-8-(4-(trifluoromethyl)phenyl)-5,6-dihydro-1,7-naphthyridine-7(8H)-carboxamide.

### Icilin Biochemical Challenge Model:

Icilin was initially developed as a “super-cooling” compound by Delmar Chemicals Ltd. In initial testing it was found to cause “wet-dog” shakes in rats. Similar shaking behavior was also evident in mice, rabbits, cats, dogs and monkeys. We set out to further characterize the in vivo actions of icilin in a rat model of spontaneous shaking behavior, also known as “wet-dog” shakes. Male Sprague Dawley rats (275-500 g, Harlan, n=4-6/treatment) were administered icilin in 2% HPMC/1% HPbCD at the following doses: 0.1, 0.3, 1.0, 3.0, 10.0 mg/kg, i.p.; 0.32, 1.0, 3.2, 10, 32 mg/kg, p.o. Spontaneous wet dog shakes were counted over 30 minutes post-icilin. Various Amgen Inc. compounds were tested (i.v., p.o.) to assess the ability to block the spontaneous wet dog shake phenomena induced by Icilin.

### CCI Model

Surgery—A chronic constriction injury (CCI) was produced as previously described (Bennett & Xie, 1988). Briefly, under gaseous anesthesia with a mixture of isoflurane (3% for induction and 2% for maintenance) in O₂, sciatic nerve was exposed at the mid-thigh level proximal to the sciatic trifurcation. Four chromic gut ligatures (4-0) were tied loosely around nerve, 1-2 mm apart such that the vascular supply was not compromised.

Behavioral testing—A behavioral test was performed to estimate cold-induced ongoing pain as previously described (Choi et al., 1994). The rat was placed under a transparent plastic cover on an aluminum plate (IITC PE34, Woodland, Calif.) which was kept at a cold temperature (5±0.5° C.). After 2 minutes of adaptation, the cumulative duration of time that the rat lifted the foot off the plate for the next 5 minutes was measured. Foot lifts associated with locomotion or grooming were not counted. Seven to 9 days after the CCI surgery, baseline of the cold-induced ongoing pain was measured. Any rat showing a cold-induced ongoing pain less than 100 sec out of 300 sec observation period was eliminated from the study. Twenty four hours after the baseline measurement, test compound, positive control, morphine (2 mg/kg, Sigma, St. Louis) or a vehicle (saline or 2% HPMC/1% Tween 80) was administered orally (test compound) or subcutaneously (morphine). Two hrs (test compound) or 30 mins (morphine) after the drug administration, the cold-induced ongoing pain was measured again.

### Chung Model

Surgery—Spinal nerve ligation surgery was performed as previously described (Kim & Chung, 1992). Briefly, under gaseous anesthesia with a mixture of isoflurane (3% for induction and 2% for maintenance) in O₂, the spinal nerve injury was produced by ligating the left L5 and L6 spinal nerves taking special care to avoid any possible damage to the L4 spinal nerve or surrounding area. Additional treatments were performed to increase the development of mechanical allodynia. First, L5 spinal nerve was cut approximately 1 mm distal to the suture as described by Li et al. (2000). Second, immediately after ligation and cut, the L4 spinal nerve was lightly manipulated by slightly stretching it with a fine hooked glass rod and gently sliding the hook back and forth 20 times along the nerve as described by Lee et al. (2003). The whole surgery procedure from anesthesia to the clipping of the incised skin took at most 15 minutes.

Behavioral testing—Two weeks later, mechanical sensitivity was measured by determining the median 50% foot withdrawal threshold for von Frey filaments using the up-down method (Chaplan et al., 1994). The rats were placed under a plastic cover (9×9×20 cm) on a metal mesh floor. The area tested was the middle glabrous area between the footpads of the plantar surface of the injured hind paw. The plantar area was touched with a series of 9 von Frey hairs with approximately exponentially incremental bending forces (von Frey values: 3.61, 3.8, 4.0, 4.2, 4.41, 4.6, 4.8, 5.0 and 5.2; equivalent to: 0.41, 0.63, 1.0, 1.58, 2.51, 4.07, 6.31, 10 and 15.8 g). The von Frey hair was presented perpendicular to the plantar surface with sufficient force to cause slight bending, and held for approximately 3-4 seconds. Abrupt withdrawal of the foot (paw flinching, shaking or licking for more than 1 sec.) was recorded as a response. Any rat showing a mechanical threshold of more than 3.16 g or less than 0.7 g after surgery was eliminated from the study. After measuring basal threshold, test compound, positive control gabapentin (Sigma, St. Louis) or a vehicle (saline or 2% HPMC/1% Tween 80) was administered orally (test compound) or intraperitoneally (gabapentin). The measurement of the tactile threshold was reassessed at 1.5 and 2 hrs after drug administration.

Data—Since the von Frey filament set was calibrated on a logarithmic scale by the vendor (Stoelting) and our selection of 9 filaments for the up-down method was also based on near equal logarithmic intervals (Dixon et al., 1980), data were treated using logarithmic values in every aspect (statistical treatment as well as plotting). However, an equivalent gram value scale is labeled on the Y-axis of the figures for convenience. Data are expressed as mean± standard error of the mean (S.E.M.).

For the treatment of vanilloid-receptor-diseases, such as acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, the compounds of the present invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition of either to a subject (i.e., an animal, preferably a mammal, most preferably a human) believed to be in need of preventative treatment, such as, for example, pain, inflammation and the like.

The dosage regimen for treating vanilloid-receptor-mediated diseases, cancer, and/or hyperglycemia with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

Compounds of the present invention can possess one or more asymmetric carbon atoms and are thus capable of existing in the form of optical isomers as well as in the form of racemic or non-racemic mixtures thereof. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using active starting materials. These isomers may be in the form of a free acid, a free base, an ester or a salt.

Likewise, the compounds of this invention may exist as isomers, that is compounds of the same molecular formula but in which the atoms, relative to one another, are arranged differently. In particular, the alkylene substituents of the compounds of this invention, are normally and preferably arranged and inserted into the molecules as indicated in the definitions for each of these groups, being read from left to right. However, in certain cases, one skilled in the art will appreciate that it is possible to prepare compounds of this invention in which these substituents are reversed in orientation relative to the other atoms in the molecule. That is, the substituent to be inserted may be the same as that noted above except that it is inserted into the molecule in the reverse orientation. One skilled in the art will appreciate that these isomeric forms of the compounds of this invention are to be construed as encompassed within the scope of the present invention.

The compounds of the present invention can be used in the form of salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methansulfonate, nicotinate, 2-naphthalenesulfonate, oxalate, palmoate, pectinate, persulfate, 2-phenylpropionate, picrate, pivalate, propionate, succinate, tartrate, thiocyanate, tosylate, mesylate, and undecanoate. Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to from pharmaceutically acceptable acid addition salts include such inorganic acids as hydrochloric acid, sulfuric acid and phosphoric acid and such organic acids as oxalic acid, maleic acid, succinic acid and citric acid. Other examples include salts with alkali metals or alkaline earth metals, such as sodium, potassium, calcium or magnesium or with organic bases.

Also encompassed in the scope of the present invention are pharmaceutically acceptable esters of a carboxylic acid or hydroxyl containing group, including a metabolically labile ester or a prodrug form of a compound of this invention. A metabolically labile ester is one which may produce, for example, an increase in blood levels and prolong the efficacy of the corresponding non-esterified form of the compound. A prodrug form is one which is not in an active form of the molecule as administered but which becomes therapeutically active after some in vivo activity or biotransformation, such as metabolism, for example, enzymatic or hydrolytic cleavage. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use. Esters of a compound of this invention, may include, for example, the methyl, ethyl, propyl, and butyl esters, as well as other suitable esters formed between an acidic moiety and a hydroxyl containing moiety. Metabolically labile esters, may include, for example, methoxymethyl, ethoxymethyl, iso-propoxymethyl, α-methoxyethyl, groups such as α-((C₁-C₄)-alkyloxy)ethyl, for example, methoxyethyl, ethoxyethyl, propoxyethyl, iso-propoxyethyl, etc.; 2-oxo-1,3-dioxolen-4-ylmethyl groups, such as 5-methyl-2-oxo-1,3,dioxolen-4-ylmethyl, etc.; C₁-C₃ alkylthiomethyl groups, for example, methylthiomethyl, ethylthiomethyl, isopropylthiomethyl, etc.; acyloxymethyl groups, for example, pivaloyloxymethyl, α-acetoxymethyl, etc.; ethoxycarbonyl-1-methyl; or α-acyloxy-α-substituted methyl groups, for example α-acetoxyethyl.

Further, the compounds of the invention may exist as crystalline solids which can be crystallized from common solvents such as ethanol, N,N-dimethyl-formamide, water, or the like. Thus, crystalline forms of the compounds of the invention may exist as polymorphs, solvates and/or hydrates of the parent compounds or their pharmaceutically acceptable salts. All of such forms likewise are to be construed as falling within the scope of the invention.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

We Claim:

1. A compound having the structure: 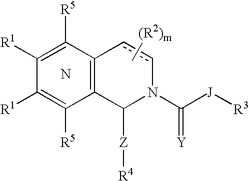
or any pharmaceutically-acceptable salt thereof, wherein: 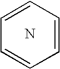
represents a six-membered heteroaryl ring containing 1 or 2 N atoms;
Y is NR^{a}, NCN, O or S;
Z is a direct bond, divalent C₁₋₄alk or divalent C₁₋₄haloalk; [symbol]
is a single bond or a double bond;
J is —N(R^{a})(CR^{c}R^{c})ₙ—, —O(CR^{c}R^{c})ₙ—, —S(CR^{c}R^{c})ₙ— or —(CR^{c}R^{c})ₙ—;
m is 0, 1 or 2;
n is 0, 1, 2 or 3;
R¹ is, independently in each instance, H, halo, C₁₋₆alk, C₁₋₆haloalk, NH₂, NHC₁₋₄alk, N(C₁₋₄alk)C₁₋₄alk or CN; or when attached to an N atom, R¹ is a lone pair of electrons;
R² is, independently in each instance, H, F, Cl, Br, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk or CN; or when attached to an N atom, R² is a lone pair of electrons;
R³ is C₁₋₈alk or a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the C₁₋₈alk and ring are substituted by 0, 1 or 2 oxo groups and the C₁₋₆alk and ring are additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(Rⁿ)C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};
R⁴ is a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the ring is substituted by 0, 1 or 2 oxo groups and the ring is additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁴ is C₄₋₁₂alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{h}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁴ is 4-biphenyl substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};
R⁵ is H, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁵ is C₁₋₆alk or a saturated, partially saturated or unsaturated 5-, 6- or 7-membered ring containing 0, 1, 2, 3 or 4 atoms selected from N, O and S, wherein the C₁₋₆alk and ring are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};
R^{a} is independently, at each instance, H or R^{b}; and
R^{b} is independently, at each instance, phenyl, benzyl or C₁₋₆alk, the phenyl, benzyl and C₁₋₆alk being substituted by 0, 1, 2 or 3 substituents selected from halo, C₁₋₄alk, C₁₋₃haloalk, —OC₁₋₄alk, —NH₂, —NHC₁₋₄alk, and —N(C₁₋₄alk)C₁₋₄alk;
R^{c} is independently, at each instance, H, halo, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk.

2. A compound having the structure: 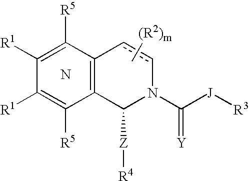
or any pharmaceutically-acceptable salt thereof, wherein: 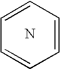
represents a six-membered heteroaryl ring containing 1 or 2 N atoms;
Y is NR^{a}, NCN, O or S;
Z is a direct bond, divalent C₁₋₄alk or divalent C₁₋₄haloalk; [symbol]
is a single bond or a double bond;
J is —N(R^{a})(CR^{c}R^{c})ₙ—, —O(CR^{c}R^{c})ₙ—, —S(CR^{c}R^{c})ₙ— or —(CR^{c}R^{c})ₙ—;
m is 0, 1 or 2;
n is 0, 1, 2 or 3;
R¹ is, independently in each instance, H, halo, C₁₋₆alk, C₁₋₆haloalk, NH₂, NHC₁₋₄alk, N(C₁₋₄alk)C₁₋₄alk or CN; or when attached to an N atom, R¹ is a lone pair of electrons;
R² is, independently in each instance, H, F, Cl, Br, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk or CN; or when attached to an N atom, R² is a lone pair of electrons;
R³ is C₁₋₈alk or a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the C₁₋₆alk and ring are substituted by 0, 1 or 2 oxo groups and the C₁₋₄alk and ring are additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};
R⁴ is a saturated, partially saturated or unsaturated 5-, 6- or 7-membered monocyclic or 8, 9, 10 or 11-membered bicyclic ring containing 0, 1, 2, 3 or 4 atoms selected from N, O and S, wherein the ring is substituted by 0, 1 or 2 oxo groups and the ring is additionally substituted by 0, 1, 2 or 3 substituents selected from C₁₋₈-alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or R⁴ is C₄₋₁₂alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};
R⁵ is H, halo, cyano, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}; or
R⁵ is C₁₋₆alk or a saturated, partially saturated or unsaturated 5-, 6- or 7-membered ring containing 0, 1, 2, 3 or 4 atoms selected from N, O and S, wherein the C₁₋₆alk and ring are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a};
R^{a} is independently, at each instance, H or R^{b}; and
R^{b} is independently, at each instance, phenyl, benzyl or C₁₋₆alk, the phenyl, benzyl and C₁₋₆alk being substituted by 0, 1, 2 or 3 substituents selected from halo, C₁₋₄alk, C₁₋₃haloalk, —OC₁₋₄alk, —NH₂, —NHC₁₋₄alk, and —N(C₁₋₄alk)C₁₋₄alk;
R^{C} is independently, at each instance, H, halo, C₁₋₄alk, C₁₋₄haloalk, —OC₁₋₄alk, —OC₁₋₄haloalk, —NH₂, —NHC₁₋₄alk or —N(C₁₋₄alk)C₁₋₄alk.

3. A compound according to claim 2, wherein R^{J} is C₁₋₈alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

4. A compound according to Claim 2, wherein R³ is phenyl substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

5. A compound according to Claim 2, wherein R³ is pyridyl or pyrimidinyl, both of which are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

6. A compound according to Claim 2, wherein R⁴ is phenyl substituted by 1, 2 or 3 substituents selected from C₁₋₆alk, C—,haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

7. A compound according to Claim 2, wherein R⁴ is phenyl substituted in para position by one substituent selected from C₁₋₆alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

8. A compound according to Claim 2, wherein R⁴ saturated, partially saturated or unsaturated 5-, 6- or 7-membered monocyclic or 8, 9, 10 or 11-membered bicyclic ring containing 1, 2, 3 or 4 atoms selected from N, O and S, wherein the ring is substituted by 0, 1 or 2 oxo groups and the ring is additionally substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}Rⁿ, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

9. A compound according to Claim 2, wherein R¹ pyridine or pyrimidine both of which are substituted by 0, 1, 2 or 3 substituents selected from C₁₋₈alk, C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a} and —NR^{a}C₂₋₆alkOR^{a}.

10. A compound according to Claim 2, wherein R⁴ is C₁₋₁₂alk substituted by 0, 1 or 2 oxo groups and additionally substituted by 0, 1, 2 or 3 substituents selected from C₁₋₄haloalk, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{h}, —N(R^{a})S(═O)₂NR^{a}R^{a}, —NR^{a}C₂₋₆alkNR^{a}R^{a} and —NRⁿC₂₋₆alkOR^{a}.

11. A compound according to Claim 2, wherein Z is a direct bond.

12. A compound selected from the group of:
(5R)—N-(4-Fluorophenyl)-5-(4-(trifluoromethyl)phenyl)-7,8-dihydro-1,6-naphthyridine-6(5H)-carboxamide;
(R)—N-((1S,2S)-2-phenylcyclopropyl)-8-(4-(trifluoromethyl)phenyl)-5,6-dihydro-1,7-naphthyridine-7(8H)-carboxamide;
(R)—N—((R)-1-phenylethyl)-8-(4-(trifluoromethyl)phenyl)-5,6-dihydro-1,7-naphthyridine-7(8H)-carboxamide;
(R)—N—((S)-1-phenylethyl)-8-(4-(tri fluoromethyl)phenyl)-5,6-dihydro-1,7-naphthyridine-7(8H)-carboxamide;
(R)—N-(2-fluorophenyl)-8-(4-(tri fluoromethyl)phenyl)-5,6-dihydro-1,7-naphthyridine-7(8H)-carboxamide;
(R)—N-(2-methoxyphenyl)-8-(4-(trifluoromethyl)phenyl)-5,6-dihydro-1,7-naphthyridine-7(8H)-carboxamide;
(R)—N-(3,4-difluorophenyl)-8-(4-(trifluoromethyl)phenyl)-5,6-dihydro-1,7-naphthyridine-7(8H)-carboxamide;
(R)—N-(3,5-dimethylisoxazol-4-yl)-8-(4-(trifluoromethyl)phenyl)-5,6-dihydro-1,7-naphthyridine-7(8H)-carboxamide;
(R)—N-(3-fluorophenyl)-8-(4-(trifluoromethyl)phenyl)-5,6-dihydro-1,7-naphthyridine-7(8H)-carboxamide;
(R)—N-(4-(dimethylamino)phenyl)-8-(4-(tri fluoromethyl)phenyl)-5,6-dihydro-1,7-naphthyridine-7(8H)-carboxamide;
(R)—N-(4-biphenyl)-8-(4-(trifluoromethyl)phenyl)-5,6-dihydro-1,7-naphthyridine-7(8H)-carboxamide;
(R)—N-(4-chlorophenyl)-8-(4-(trifluoromethyl)phenyl)-5,6-dihydro-1,7-naphthyridine-7(8H)-carboxamide;
(R)—N-(4-cyanophenyl)-8-(4-(trifluoromethyl)phenyl)-5,6-dihydro-1,7-naphthyridine-7(8H)-carboxamide;
(R)—N-(4-Fluorophenyl)-5-(4-(trifluoromethyl)phenyl)-7,8-dihydropyrido[3,4-b]pyrazine-6(5H)-carboxamide;
(R)—N-(4-Fluorophenyl)-8-(4-(trifluoromethyl)phenyl)-5,6-dihydro-1,7-naphthyridine-7(8H)-carboxamide;
(R)—N-(4-methoxyphenyl)-8-(4-(trifluoromethyl)phenyl)-5,6-dihydro-1,7-naphthyridine-7(8H)-carboxamide;
(R)—N-(benzo[d][1,3]dioxol-5-yl)-8-(4-(trifluoromethyl)phenyl)-5,6-dihydro-1,7-naphthyridine-7(8H)-carboxamide;
(R)—N-(pyridin-2-yl)-8-(4-(trifluoromethyl)phenyl)-5,6-dihydro-1,7-naphthyridine-7(8H)-carboxamide;
(R)—N-(pyridin-3-yl)-8-(4-(trifluoromethyl)phenyl)-5,6-dihydro-1,7-naphthyridine-7(8H)-carboxamide;
(R)—N-benzyl-8-(4-(trifluoromethyl)phenyl)-5:6-dihydro-1,7-naphthyridine-7(8H)-carboxamide;
(R)—N-phenethyl-8-(4-(trifluoromethyl)phenyl)-5,6-dihydro-1,7-naphthyridine-7(8H)-carboxamide;
(R)—N-tert-butyl-8-(4-(trifluoromethyl)phenyl)-5,6-dihydro-1,7-naphthyridine-7(8H)-carboxamide;
(S)—N-(4-Fluorophenyl)-5-(4-(trifluoromethyl)phenyl)-7)8-dihydropyrido[3,4-b]pyrazine-6(5H)-carboxamide;
(S)—N-(4-Fluorophenyl)-8-(4-(trifluoromethyl)phenyl)-5,6-dihydro-1,7-naphthyridine-7(8H)-carboxamide;
(S)—N-(pyridin-3-yl)-8-(4-(trifluoromethyl)phenyl)-5,6-dihydro-1,7-naphthyridine-7(8H)-carboxamide;
Ethyl 5-(4-(trifluoromethyl)phenyl)-1,6-naphthyridine-6(5H)-carboxylate;
Ethyl 5-(4-(trifluoromethyl)phenyl)pyrido[3,4-b]pyrazine-6(5H)-carboxylate;
Ethyl 8-(4-(trifluoromethyl)phenyl)-1,7-naphthyridine-7(8H)-carboxylate;
N-(4-Fluorophenyl)-1-(4-(trifluoromethyl)phenyl)-3,4-dihydro-2,7-naphthyridine-2(1H)-carboxamide;
N-(4-Fluorophenyl)-1-(4-(trifluoromethyl)phenyl)-3,4-dihydro-2,6-naphthyridine-2(1H)-carboxamide;
N-(4-Fluorophenyl)-5-(4-(trifluoromethyl)phenyl)-7,8-dihydro-1,6-naphthyridine-6(5H)-carboxamide;
N-(4-Fluorophenyl)-5-(4-(trifluoromethyl)phenyl)-7,8-dihydropyrido[354-b]pyrazine-6(5M)-carboxamide;
N-(4-Fluorophenyl)-8-(4-(trifluoromethyl)phenyl)-5,6-dihydro-1,7-naphthyridine-7(8H)-carboxamide;
N-(4-Fluorophenyl)-8-(4-(trifluoromethyl)phenyl)-5,6-dihydropyrido[3,4-d]pyrimidine-7(8H)-carboxamide;
(R)—N-Isopropyl-5-(4-(trifluoromethyl)phenyl)-7,8-dihydropyrido[3,4-b]pyrazine-6(5H)-carboxamide;
(R)—N-(Pyridin-3-yl)-5-(4-(trifluoromethyl)phenyl)-7,8-dihydropyrido[3,4-b]pyrazine-6(5H)-carboxamide;
(R)—N,8-Bis(4-(trifluoromethyl)phenyl)-5,6-dihydro-1,7-naphthyridine-7(8H)-carboxamide;
(R)—N-(2-Cyanophenyl)-8-(4-(trifluoromethyl)phenyl)-5,6-dihydro-1,7-naphthyridine-7(8H)-carboxamide;
(R)—N-(3-Cyanophenyl)-8-(4-(trifluoromethyl)phenyl)-5,6-dihydro-1,7-naphthyridine-7(8H)-carboxamide;
Benzyl 8-(4-(trifluoromethyl)phenyl)-1,7-naphthyridine-7(8H)-carboxylate;
Ethyl 8-(4-fluorophenyl)-1,7-naphthyridine-7(8H)-carboxylate;
Ethyl 8-(4-fluorophenyl)-5,6-dihydro-1,7-naphthyridine-7(8H)-carboxylate;
N,8-Bis(4-fluorophenyl)-5,6-dihydro-1,7-naphthyridine-7(8H)-carboxamide;
N-(4-Fluorophenyl)-8-(4-biphenyl)-5,6-dihydro-1,7-naphthyridine-7(8H)-carboxamide;
(6,8)-N-(4-Fluorophenyl)-6-methyl-8-(4-(trifluoromethyl)phenyl)-5,6-dihydro-1,7-naphthyridine-7(8H)-carboxamide (racemic);
(5,8)-N-(4-Fluorophenyl)-5-methyl-8-(4-(trifluoromethyl)phenyl)-5,6-dihydro-1,7-naphthyridine-7(8H)-carboxamide (racemic);
(R)—N,4-bis(4-Fluorophenyl)-8-(4-(trifluoromethyl)phenyl)-5,6-dihydro-1,7-naphthyridine-7(8H)-carboxamide; and
(R)—N-(4-fluorophenyl)-4-methyl-8-(4-(trifluoromethyl)phenyl)-5,6-dihydro-1,7-naphthyridine-7(8H)-carboxamide;
or any pharmaceutically-acceptable salt thereof.

13. A method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, comprising the step of administering a compound according to claim 1.

14. A pharmaceutical composition comprising a compound according to claim 1 and a pharmaceutically-acceptable diluent or carrier.
